(19) United States
(12) Patent Application Publication
(10) Pub. No.: US 2025/0019755 A1
(43) Pub. Date: Jan. 16, 2025

(54) METHODS AND MATERIALS FOR ASSESSING NUCLEIC ACIDS

(71) Applicant: The Johns Hopkins University, Baltimore, MD (US)

(72) Inventors: Nickolas Papadopoulos, Towson, MD (US); Kenneth W. Kinzler, Frankford, DE (US); Bert Vogelstein, Baltimore, MD (US); Joshua David Cohen, Baltimore, MD (US)

(21) Appl. No.: 18/899,799

(22) Filed: Sep. 27, 2024

Related U.S. Application Data
(63) Continuation of application No. 17/174,838, filed on Feb. 12, 2021.
(60) Provisional application No. 62/977,066, filed on Feb. 14, 2020.

(51) Int. Cl. C12Q 1/6855; C12Q 1/6876

(52) U.S. Cl. CPC C12Q 1/6855; C12Q 1/6876; C12Q 2563/185

(57) ABSTRACT

Provided herein are systems, kits, compositions and methods for sequencing library preparation and sequencing workflow (e.g., for the identification of mutations). In certain embodiments, provides herein systems and methods to identically barcode both strands of templates, and PCR-based enrichment of each strand that does not require hybridization capture.

The present application is a continuation of U.S. application Ser. No. 17/174,838, filed on Feb. 12, 2021, which claims priority to U.S. Provisional Application Ser. No. 62/977,066, filed Feb. 14, 2020, which are herein incorporated by reference in their entireties.

### STATEMENT REGARDING FEDERAL FUNDING

This invention was made with government support under grants CA062924, CA152753 and CA230691 awarded by the National Institutes of Health. The government has certain rights in the invention.

### TECHNICAL FIELD

This invention is related to the area of nucleotide sequencing. In particular, it relates to sequencing library preparation and sequencing workflow for the identification of mutations.

### SEQUENCE LISTING

This application contains a Sequence Listing that has been submitted electronically as an XML file named “44807-0341002_ST26_SL.XML.” The XML file, created on Jun. 5, 2024, is 105,675 bytes in size. The material in the XML file is hereby incorporated by reference in its entirety.

### BACKGROUND INFORMATION

The identification of rare mutations is useful in aspects of fundamental biological as well as to improve the clinical management of patients. Fields of use include infectious diseases, immune repertoire profiling, palentogenetics, forensics, aging, non-invasive prenatal testing, and cancer. Next generation sequencing (NGS) technologies are theoretically suitable for this application, and a variety of NGS approaches exist for the detection of rare mutations. However, for conventional NGS approaches, the error rate of the sequencing itself is too high to allow confident detection of mutations, particularly those mutations present at low frequencies in the original sample.

The use of molecular barcodes to tag original template molecules was designed to overcome various obstacles in the detection of rare mutations. With molecular barcoding, redundant sequencing of the PCR-generated progeny of each tagged molecule is performed and sequencing errors are easily recognized (Kinde et al., Proc Natl Acad Sci USA 108:9530-9535 (2011)). For example, if a given threshold of the progeny of the barcoded template molecule contain the same mutation, then the mutation is considered genuine (a “supermutant”). If less than a given threshold of the progeny contain the mutation of interest, then the mutation is considered an artifact. Two types of molecular barcodes have been described: exogenous and endogenous. Exogenous barcodes (also referred to herein as exogenous UIDs) comprise pre-specified or random nucleotides, and are appended during library preparation or during PCR. Endogenous barcodes (also referred to herein as endogenous UIDS) are formed by the sequences at the 5′ and 3′ ends of fragments (e.g., fragments generated by random shearing of DNA or fragments present in a cell-free fluid biological sample) of the template DNA to be assayed. Such barcodes have been proven useful for tracing amplicons back to an original starting template, allowing for molecular counting and improving the identification of true mutations in clinically-relevant samples.

Forked adapters for paired end sequencing have been developed to enable “duplex sequencing”, wherein each of the two strands (Watson and Crick) of the original DNA duplex can be discerned by the 5′ to 3′ directionality revealed upon sequencing. Duplex sequencing reduces sequencing errors because it is extremely unlikely that both strands of DNA would contain the identical mutation if that mutation was erroneously generated during library preparation or sequencing.

However, there are many issues that have limited the range of applications and clinical applicability of molecular barcoding. For example, it is challenging to convert a large fraction of the initial template molecules to duplex molecules with the same barcode on each strand (Schmitt et al., Proc Natl Acad Sci USA 109:14508-14513 (2012); Schmitt et al., Nat Methods 12:423-425 (2015); and Newman et al., Nat Biotechnol 34:547-555 (2016)). This issue is particularly problematic when the amount of initial DNA is limited (e.g. <33 ng), such as is found in the cell-free plasma DNA typically used for liquid biopsies.

Preparation of targeted sequencing libraries generally involve adapter attachment to sequencing templates, amplification of the library, and hybridization capture to enrich the library for targets of interest. While effective for enriching large regions of interest, hybridization capture does not scale well for small target regions (Springer et al., Elife 7:doi:10.7554/eLife.32143 (2018)) and exhibits poor duplex recovery (Wang et al., Proc Natl Acad Sci USA 112:9704-9709 (2015); and Wang et al., Elife 5:doi:10.7554/eLife.15175 (2016)). Sequential rounds of capture can partially overcome these limitations, but even with such improvements, duplex recovery rates are typically ˜1%. CRISPR-DS can achieve up to 15% recovery, but is not applicable to cell-free DNA. When the targeted region is very small (e.g. one or a few positions in the genome of particular interest, such as required for disease monitoring in plasma), or the amount of DNA available is limited (e.g. <33 ng, as often found in plasma), capture-based approaches are suboptimal.

Accordingly, there exists a need for improvements to sequencing library preparation and workflow, to enable accurate identification of mutations, e.g., rare mutations, in clinically relevant samples such as liquid biopsy samples.

### SUMMARY

The present document provides methods and materials that addresses these issues by providing an approach to identically barcode both strands of templates, and by providing a method for PCR-based enrichment of each strand that does not require hybridization capture.

This document relates to methods and materials that can be used to detect the presence of one or more mutations present on both strands of a double stranded nucleic acid (e.g., DNA). In some cases, methods for detecting one or more mutations present on both strands of a double stranded nucleic acid can include generating a duplex sequencing library having a duplex molecular barcode on each end (e.g., the 5′ end and the 3′ end) of each nucleic acid in the library, generating a library of single stranded Watson strand-derived sequences and a library of single stranded Crick-strand derived sequences from the duplex sequencing library, and detecting the presence of one or more mutations present on both strands of the double stranded nucleic acid in each single stranded library.

As demonstrated herein, a single stranded DNA library corresponding to the Watson strands of a double stranded nucleic acid template and a single stranded DNA library corresponding to the Crick strands of a double stranded nucleic acid template can be generated from sequencing libraries incorporating duplex molecular barcodes, each single stranded DNA library can be enriched for a target region using a strand-specific anchored PCR approach, and the target region can be sequenced to detect the presence of absence of one or more mutations within the target region of a nucleic acid. For example, methods and materials described herein that can be used to detect the presence of one or more mutations present on both strands of a double stranded nucleic acid can be termed the Sequence Ascertainment Free of Errors Sequencing System (SaferSeqS), and can include for example, library construction with in situ generation of double stranded molecular barcodes (see, e.g., FIG. 22a), target enrichment via anchored PCR (see, e.g., FIG. 22b), and in silico reconstruction of template molecules (see, e.g., FIG. 22c). Bonafide mutations present in the original starting templates can be identified by requiring alterations to be found on both strands of the same initial nucleic acid molecule.

Having the ability to detect one or more mutations present on both strands of a double stranded nucleic acid (e.g., true somatic mutations) provides a unique and unrealized opportunity to accurately and efficiently assess multiple mutations simultaneously and at an affordable cost. Using methods and materials described herein to detect the presence of one or more mutations present on both strands of a double stranded nucleic acid (e.g., SaferSeqS methods) can permit the identification of rare mutations with high confidence, while minimizing the amount of DNA damage, the amount of PCR to be performed, and/or the number of DNA damage artifacts. It is noted that the terms “Watson strand” and “Crick strand” are used simply to distinguish between the two strands of a double-stranded starting nucleic acid sequence. Either of the strands can be denoted “Watson” or “Crick,” and then the other strand is denoted with the other name.

In some embodiments, provided herein are methods of comprising: a) forming a reaction mixture comprising: i) a plurality of double-stranded DNA fragments that are dephosphorylated and blunt-ended, wherein each of the double-stranded DNA fragments comprises Watson and Crick strands; ii) a plurality of adaptors, wherein each of the adaptors comprises, in the 5′ to 3′ direction: A) a barcode, and B) a universal 3′ adaptor sequence; and iii) a ligase; and b) incubating the reaction mixture such that: i) adaptors are ligated to the 3′ ends of the Watson and Crick strands, and ii) adaptors are not ligated to the 5′ ends of either the Watson or Crick strands, thereby generating double-stranded ligation products.

In certain embodiments, each of the plurality of adaptors comprises a unique barcode. In further embodiments, each of the double-stranded ligation products comprises a Watson strand with only one barcode and a Crick strand with only one barcode that is different from the barcode on the Watson strand. In further embodiments, the methods further comprise the step of: c) sequencing at least a portion of the double-stranded ligation products.

In particular embodiments, provided herein are methods comprising: a) attaching partially double-stranded 3′ adaptors (3′PDSAs) to 3′ ends of both Watson and Crick strands of a population of double-stranded DNA fragments in an analyte DNA sample, wherein a first strand of the 3′PDSAs comprises, in the 5′-3′ direction, (i) a first segment, (ii) an exogenous UID sequence, (iii) an annealing site for a 5′ adaptor, and (iv) a universal 3′ adaptor sequence comprising an R2 sequencing primer site, and wherein the second strand of the 3′PDSAs comprises, in the 5′ to 3′ direction, (i) a segment complementary to the first segment, and, optionally, (ii) a 3′ blocking group, b) annealing 5′ adaptors to the annealing site, wherein the 5′ adaptors comprise, in the 5′ to 3′ direction, (i) a universal 5′ adaptor sequence that is not complementary to the universal 3′ adaptor sequence and that comprises an R1 sequencing primer site, and (ii) a sequence complementary to the annealing site for the 5′ adaptor; c) extending the 5′ adaptors across the exogenous UID sequence and the first segment, thereby generating the complement of the exogenous UID sequence and complement of the first segment, and d) covalently linking the 3′ end of the complement of the first segment to the 5′ ends of the Watson and Crick strands of the double-stranded DNA fragments, thereby generating a plurality of adaptor-ligated double-stranded DNA fragments.

In some embodiments, provided herein are methods comprising: a) attaching partially double-stranded 3′ adaptors (3′PDSAs) to 3′ ends of both Watson and Crick strands of a population of double-stranded DNA fragments in an analyte DNA sample, wherein a first strand of the 3′PDSAs comprises, in the 5′-3′ direction, (i) a first segment, (ii) an exogenous UID sequence, (iii) an annealing site for a 5′ adaptor, and (iv) a universal 3′ adaptor sequence comprising an R2 sequencing primer site, and wherein the second strand of the 3′PDSAs comprises, in the 5′ to 3′ direction, (i) a segment complementary to the first segment, and optionally (ii) a 3′ blocking group, b) annealing 5′ adaptors to the annealing site, wherein the 5′ adaptors comprise, in the 5′ to 3′ direction, (i) a universal 5′ adaptor sequence that is not complementary to the universal 3′ adaptor sequence and that comprises an R1 sequencing primer site, and (ii) a sequence complementary to the annealing site for the 5′ adaptor; c) extending the 5′ adaptors across the exogenous UID sequence, thereby generating the complement of the exogenous UID sequence, and d) covalently linking the 3′ end of the complement of the exogenous UID sequence to the 5′ ends of the segment complementary to the first segment on each of the Watson and Crick strands of the double-stranded DNA fragments, thereby generating a plurality of adaptor-ligated double-stranded DNA fragments.

In some embodiments, provided herein are methods comprising: a) attaching partially double-stranded 3′ adaptors (3′PDSAs) to 3′ ends of both Watson and Crick strands of a population of double-stranded DNA fragments in an analyte DNA sample, wherein a first strand of the 3′PDSAs comprises, in the 5′-3′ direction, (i) a first segment, (ii) an exogenous UID sequence, (iii) an annealing site for a 5′ adaptor, and (iv) a universal 3′ adaptor sequence comprising an R2 sequencing primer site, and wherein the second strand of the 3′PDSAs comprises, in the 5′ to 3′ direction, (i) a segment complementary to the first segment, and optionally (ii) a 3′ blocking group, b) annealing 5′ adaptors to the annealing site, wherein the 5′ adaptors comprise, in the 5′ to 3′ direction, (i) a universal 5′ adaptor sequence that is not complementary to the universal 3′ adaptor sequence and that comprises an R1 sequencing primer site, and (ii) a sequence complementary to the annealing site for the 5′ adaptor; c) extending the 5′ adaptors across the exogenous UID sequence and the first segment of the 3′PDSA, thereby generating the complement of the exogenous UID sequence and the complement of the first segment of the 3′PDSA, and d) covalently linking the 3′ end of the complement of the first segment of the 3′PDSA to the 5′ ends of the Watson and Crick strands of the double-stranded DNA fragments, thereby generating a plurality of adaptor-ligated double-stranded DNA fragments.

In some embodiments, provided here are systems and kits and compositions comprising: a) a population of partially double-stranded 3′ adaptors (3′PDSAs) configured to be ligated to the 3′ ends of both Watson and Crick strands of a population of double-stranded DNA fragments, wherein a first strand of the 3′PDSAs comprises, in the 5′-3′ direction, (i) a first segment, (ii) an exogenous UID sequence, (iii) an annealing site for a 5′ adaptor, and (iv) a universal 3′ adaptor sequence comprising an R2 sequencing primer site, and wherein the second strand of the 3′PDSAs comprises, in the 5′ to 3′ direction, (i) a segment complementary to the first segment, and (ii) a 3′ blocking group; and b) a population of 5′ adaptors configured to anneal to the annealing site, wherein the 5′ adaptors comprise, in the 5′ to 3′ direction, (i) a universal 5′ adaptor sequence that is not complementary to the universal 3′ adaptor sequence and that comprises an R1 sequencing primer site, and (ii) a sequence complementary to the annealing site for the 3′ adaptor.

In further embodiments, the systems, kits, and compositions further comprise: c) the population of double-stranded DNA fragments from a biological sample, and/or c) reagents for degrading the second strand of the 3′PDSAs to generate single-stranded 3′ adaptors (3′SSAs); and/or c) a first primer complementary to the universal 3′ adaptor sequence, and second primer complementary to the complement of the universal 5′ adaptor sequence; and/or c) a sequencing system; and/or c) a Watson anchor primer complimentary to the universal 3′ adapter sequence, and d) a Crick anchor primer complimentary to the complement of the universal 5′ adapter sequence; and/or c) a first set of Watson target-selective primer pairs, comprising (i) one or more first Watson target-selective primers comprising a sequence complementary to a portion of the universal 3′ adapter sequence, and (ii) one or more second Watson target selective primers, each of the one or more second Watson target-selective primers comprising a target-selective sequence; and/or c) a first set of Crick target-selective primer pairs, comprising (i) one or more Crick target-selective primers comprising a sequence complementary to a portion of the universal 5′ adapter sequence, and (ii) one or more second Crick target-selective primers, each of the one or more second Crick target selective primers comprising the same target-selective sequence as the second Watson target-selective primer sequence.

In some embodiments, the methods further comprise: amplifying the plurality of adaptor-ligated, double-stranded DNA fragments with a first primer complementary to the universal 3′ adaptor sequence, and second primer complementary to the complement of the universal 5′ adaptor sequence, thereby generating amplicons, wherein the amplicons comprise a plurality of double-stranded Watson templates and a plurality of double-stranded Crick templates. In certain embodiments, the methods further comprise: selectively amplifying said double-stranded Watson templates with a first set of Watson target-selective primer pairs, the first set of Watson target-selective primer pairs comprising: (i) a first Watson target-selective primer comprising a sequence complementary to a portion of the universal 3′ adapter sequence, and (ii) a second Watson target-selective primer comprising a target-selective sequence, thereby creating target Watson amplification products. In further embodiments, the methods further comprise: selectively amplifying said double-stranded Crick templates with a first set of Crick target-selective primer pairs, the first set of Crick target-selective primer pairs comprising: (i) a first Crick target-selective primer comprising a sequence complementary to the complement of a portion of the universal 5′ adapter sequence, and (ii) a second Crick target-selective primer comprising the same target-selective sequence as the second Watson target-selective primer sequence, thereby creating target Crick amplification products. In certain embodiments, the population of double-stranded DNA fragments are incubated with a mixture of uracil-DNA glycosylase and DNA glycosylase-lyase Endonuclease VIII, prior to ligating any adapters.

In some embodiments, the polymerase employed (e.g., for extending the 5′ adapter sequences) has 5′ to 3′ exonuclease activity (e.g., which can digest the second strand of the 3′PDSAs). In other embodiments, the polymerase employed (e.g., for extending the 5′ adapter sequences) does not have 5′ to 3′ exonuclease activity.

In other embodiments, the methods further comprise: removing the second strand of the 3′PDSAs to generate single-stranded 3′ adaptors (3′SSAs). In other embodiments, wherein the removing the second strand occurs after step b), or before step b), or during step b). In some embodiments, the removing the second strand of the 3′PDSAs comprises contacting the 3′ duplex adapter with uracil-DNA glycosylase (UDG) to degrade the second strand. In further embodiments, the removing the second strand is accomplished by a polymerase with exonuclease activity, wherein the polymerase extends the 5′ adaptors across the exogenous UID sequence and the first segment.

In further embodiments, the methods further comprise: determining sequence reads of one or more of the amplicons. In other embodiments, the methods further comprise: assigning the sequence reads into UID families, wherein each member of a UID family comprises the same exogenous UID sequence. In particular embodiments, the methods further comprise: assigning sequence reads of each UID family into a Watson subfamily and Crick subfamily based on spatial relationship of the exogenous UID sequence to the R1 and R2 read sequence. In other embodiments, the methods further comprise: identifying a nucleotide sequence as accurately representing a Watson strand of an analyte DNA fragment when at least 50% (e.g., 50 . . . 75 . . . 95%) of the Watson subfamily contain the sequence. In other embodiments, the methods further comprise: identifying a nucleotide sequence as accurately representing a Crick strand of an analyte DNA fragment when at least 50% (e.g., 50 . . . 75 . . . 90%) of the Crick subfamily contain the sequence.

In some embodiments, the methods further comprise: identifying a mutation in the nucleotide sequence as accurately representing a the Watson strand when the sequence accurately representing the Watson strand differs from a reference sequence that lacks the mutation. In additional embodiments, the methods further comprise: identifying a mutation in the nucleotide sequence as accurately representing the Crick Strand when the sequence accurately representing the Crick Strand differs from a reference sequence that lacks the mutation. In other embodiments, the methods further comprise: identifying a mutation in the analyte DNA fragment when the mutation in the nucleotide sequence accurately representing the Watson strand and the mutation in the nucleotide sequence accurately representing the Crick strand are the same mutation. In some embodiments, each member of a UID family further comprises the same endogenous UID sequence, wherein the endogenous UID sequence comprises an end of a double-stranded DNA fragment from the population. In other embodiments, the population of double-stranded DNA fragments has blunt ends.

Provided herein are for detecting the presence or absence of a mutation in a target region of a double stranded DNA template obtained from a sample from a mammal, and determining whether the mutation is present on both strands of the double stranded DNA template, wherein the method comprises: A) generating double stranded DNA fragments each having a duplex molecular barcode on each end of the double stranded DNA fragment; B) amplifying the double stranded DNA fragment comprising the duplex molecular barcode on each end of the double stranded DNA fragment to generate an amplified duplex sequencing library, wherein the amplifying comprises contacting the double stranded DNA fragment comprising the duplex molecular barcode on each end of the double stranded DNA fragment with a universal primer pair under whole-genome PCR conditions; C) optionally, generating a single stranded DNA library of Watson strands from the amplified duplex sequencing library; D) optionally, generating a single stranded DNA library of Crick strands from the amplified duplex sequencing library; E) amplifying the target region from the DNA library (e.g., the single stranded DNA library) of Watson strands using a primer pair consisting of a first primer capable of hybridizing to the target region and a second primer capable of hybridizing to the 3′ duplex adapter; F) amplifying the target region from the DNA library (e.g., the single stranded DNA library) of Crick strands using a primer pair consisting of a first primer capable of hybridizing to the target region and a second primer capable of hybridizing to the 5′ adapter; G) sequencing the target region amplified from the DNA library (e.g., the single stranded DNA library) (e.g., the DNA library (e.g., the single stranded DNA library)) of Watson strands to generate a sequencing read and to detect the presence or absence of the mutation in a Watson strand of the target region; H) sequencing the target region amplified from the DNA library (e.g., the single stranded DNA library) (e.g., the DNA library (e.g., the single stranded DNA library)) of Crick strands to generate a sequencing read and detect the presence or absence of the mutation in a Crick strand of the target region; and I) grouping the sequencing reads by the molecular barcode present in each sequencing read to determining whether the mutation is present on both strands of the double stranded DNA template. In some embodiments, generating the double stranded DNA fragments each having the duplex molecular barcode on each end of the double stranded DNA fragment comprises: i) ligating a 3′ duplex adapter to each 3′ end of a double stranded DNA fragment obtained from the double stranded DNA template, wherein the 3′ duplex adapter comprises a) a first oligonucleotide comprising a 5′ phosphate, a first molecular barcode, and a 3′ oligonucleotide annealed to b) a second oligonucleotide comprising a degradable 3′ blocking group, wherein the 3′ oligonucleotide and the second oligonucleotide sequence are complementary; ii) degrading the degradable 3′ blocking group; iii) ligating a 5′ adapter to each dephosphorylated 5′ end of the double stranded DNA fragment obtained from the double stranded DNA template, wherein the 5′ duplex adapter comprises an oligonucleotide comprising a second molecule barcode, wherein the second molecular barcode is different from the first molecular barcode, wherein the 5′ adapter is ligated onto the double stranded DNA fragment upstream of the first molecular barcode and leaving a gap of single stranded nucleic acid between the 5′ end of the double stranded DNA fragment and the 5′ adapter; and iv) filling in the gap of single stranded nucleic acid between the 5′ end of the double stranded DNA fragment and the 5′ adapter to generate the double stranded DNA fragment comprising the duplex molecular barcode on each end of the double stranded DNA fragment. In some embodiments, generating the DNA library (e.g., the single stranded DNA library) (e.g., the DNA library (e.g., the single stranded DNA library)) of Watson strands from the amplified duplex sequencing library comprises: i) amplifying a first aliquot of the amplified duplex sequencing library using a primer pair consisting of a first primer and a second primer, wherein the first primer is capable of hybridizing to the Watson strand, and wherein the first primer comprises a tag, to generate double stranded amplification products having tagged Watson strands; ii) denaturing the double stranded amplification products having tagged Watson strands to generate single stranded tagged Watson strands and single stranded Crick strands; and iii) recovering the single stranded tagged Watson strands to generate the DNA library (e.g., the single stranded DNA library) of Watson strands from the amplified duplex sequencing library.

In some embodiments, the double stranded DNA template is obtained from a sample from a mammal the generating the DNA library (e.g., the single stranded DNA library) of Crick strands from the amplified duplex sequencing library comprises: i) amplifying a second aliquot of the amplified duplex sequencing library using a primer pair consisting of a first primer and a second primer, wherein the first primer is capable of hybridizing to the Crick strand, and wherein the first primer comprises a tag, to generate double stranded amplification products having tagged Crick strands; ii) denaturing the double stranded amplification products having tagged Crick strands to generate single stranded tagged Crick strands and single stranded Watson strands; and iii) recovering the single stranded tagged Crick strands to generate the DNA library (e.g., the single stranded DNA library) of Crick strands from the amplified duplex sequencing library. In some embodiments, the mammal is a human.

In some embodiments, the method further comprises, prior to the generating a double stranded DNA fragment having the duplex molecular barcode on each end of the double stranded DNA fragment: fragmenting the double stranded DNA to generate double stranded DNA fragments; dephosphorylating 5′ ends of the double stranded DNA fragments; and blunting the ends of double stranded DNA fragments. In some embodiments, ligating a 3′ duplex adapter to each 3′ end of a double stranded DNA fragment obtained from the double stranded DNA template comprises contacting the 3′ duplex adapter and the double stranded DNA fragment obtained from the double stranded DNA template in the presence of a ligase. In some embodiments, the ligase is a T4 DNA ligase.

In some embodiments, degrading the degradable 3′ blocking group comprises contacting the 3′ duplex adapter with uracil-DNA glycosylase (UDG). In some embodiments, ligating the 5′ adapter to each dephosphorylated 5′ end of the double stranded DNA fragment obtained from the double stranded DNA template comprises contacting the 5′ adapter and the double stranded DNA fragment obtained from the double stranded DNA template in the presence of a ligase. In some embodiments, the ligase is an Escherichia coli ligase.

In some embodiments, filling in the gap of single stranded nucleic acid between the 5′ end of the double stranded DNA fragment and the 5′ adapter comprises contacting the 5′ end of the double stranded DNA fragment and the 5′ adapter in the presence of a polymerase and dNTPs. In some embodiments, the polymerase is a Taq polymerase.

In some embodiments, the ligating the 5′ adapter to each 5′ end of the double stranded DNA fragment, and the filling in the gap between the 5′ end of the double stranded DNA fragment and the 5′ adapter are performed concurrently. In some embodiments, amplifying the double stranded DNA fragment comprising the duplex molecular barcode on each end of the double stranded DNA fragment to generate an amplified duplex sequencing library comprises contacting the double stranded DNA fragment comprising the duplex molecular barcode on each end of the double stranded DNA fragment with a universal primer pair under PCR conditions. In some embodiments, the amplifying comprises whole-genome PCR. In some embodiments, the tagged primers are biotinylated primers, and wherein the biotinylated primers can generate biotinylated single stranded Watson strands and biotinylated single stranded Crick strands. In some embodiments, the denaturing steps comprise NaOH denaturation, heat denaturation, or a combination of both.

In some embodiments, the recovering steps comprise contacting the tagged Watson strands with streptavidin-functionalized beads and contacting the tagged Crick strands with streptavidin-functionalized beads. In some embodiments, the recovering steps further comprise denaturing non-tagged Watson strands and denaturing non-tagged Watson strands. In some embodiments, the recovering steps further comprise releasing the biotinylated single stranded Watson strands from the streptavidin-functionalized beads and releasing the biotinylated single stranded Crick strands from the streptavidin-functionalized beads. In some embodiments, the tagged primers are phosphorylated primers, and the phosphorylated primers can generate phosphorylated single stranded Watson strands and phosphorylated single stranded Crick strands. In some embodiments, the denaturing steps comprises lambda exonuclease digestion.

In some embodiments, amplifying the target region from the DNA library (e.g., the single stranded DNA library) of Watson strands further comprises second amplification using a second primer pair consisting of a first primer capable of hybridizing to the target region and a second primer capable of hybridizing to the 3′ duplex adapter; and wherein the amplifying the target region from the DNA library (e.g., the single stranded DNA library) of Crick strands further comprises a second amplification using a second primer pair consisting of a first primer capable of hybridizing to the target region and a second primer capable of hybridizing to the 5′ adapter. In some embodiments, the sequencing steps comprise paired-end sequencing.

Also provided herein are methods for detecting the presence or absence of a mutation in a target region of a double stranded DNA template obtained from a sample from a mammal, and determining whether the mutation is present on both strands of the double stranded DNA template, wherein the method comprises: A) generating double stranded DNA fragments each having a duplex molecular barcode on each end of the double stranded DNA fragment; B) generating a DNA library (e.g., the single stranded DNA library) of Watson strands and a DNA library (e.g., the single stranded DNA library) of Crick strands from the amplified duplex sequencing library from the double stranded DNA fragment having the duplex molecular barcode on each end of the double stranded DNA fragment; C) amplifying the target region from the single stranded Watson strands using a primer pair consisting of a first primer capable of hybridizing to the target region and a second primer capable of hybridizing to the 3′ duplex adapter; D) amplifying the target region from the single stranded Crick strands using a primer pair consisting of a first primer capable of hybridizing to the target region and a second primer capable of hybridizing to the 5′ adapter; E) sequencing the target region amplified from the DNA library (e.g., the single stranded DNA library) of Watson strands to generate a sequencing read and to detect the presence or absence of the mutation in a Watson strand of the target region; F) sequencing the target region amplified from the DNA library (e.g., the single stranded DNA library) of Crick strands to generate a sequencing read and detect the presence or absence of the mutation in a Crick strand of the target region; and G) grouping the sequencing reads by the molecular barcode present in each sequencing read to determining whether the mutation is present on both strands of the double stranded DNA template.

In some embodiments, the double stranded DNA template is a genomic DNA sample and generating the double stranded DNA fragments each having the duplex molecular barcode on each end of the double stranded DNA fragment comprises: i) ligating a 3′ duplex adapter to each 3′ end of a double stranded DNA fragment obtained from the double stranded DNA template, wherein the 3′ duplex adapter comprises a) a first oligonucleotide comprising a 5′ phosphate, a first molecular barcode, and a 3′ oligonucleotide annealed to b) a second oligonucleotide comprising a degradable 3′ blocking group, wherein the 3′ oligonucleotide and the second oligonucleotide sequence are complementary; ii) degrading the degradable 3′ blocking group; iii) ligating a 5′ adapter to each dephosphorylated 5′ end of the double stranded DNA fragment obtained from the double stranded DNA template, wherein the 5′ duplex adapter comprises an oligonucleotide comprising a second molecule barcode, wherein the second molecular barcode is different from the first molecular barcode, wherein the 5′ adapter is ligated onto the double stranded DNA fragment upstream of the first molecular barcode and leaving a gap of single stranded nucleic acid between the 5′ end of the double stranded DNA fragment and the 5′ adapter; and iv) filling in the gap of single stranded nucleic acid between the 5′ end of the double stranded DNA fragment and the 5′ adapter to generate the double stranded DNA fragment comprising the duplex molecular barcode on each end of the double stranded DNA fragment.

In some embodiments, the double stranded DNA template is a cell-free DNA sample and generating the DNA library (e.g., the single stranded DNA library) of Watson strands and the DNA library (e.g., the single stranded DNA library) of Crick strands from the amplified duplex sequencing library from the double stranded DNA fragment having the duplex molecular barcode on each end of the double stranded DNA fragment comprises: i) amplifying the double stranded DNA fragment having the duplex molecular barcode on each end of the double stranded DNA fragment using a universal primer pair consisting of a first primer and a second primer, wherein the amplifying comprises contacting the double stranded DNA fragment comprising the duplex molecular barcode on each end of the double stranded DNA fragment with the primer pair under whole-genome PCR conditions, wherein the first primer is capable of hybridizing to the Watson strand, and wherein the first primer is biotinylated, to generate double stranded amplification products having biotinylated Watson strands; ii) contacting the double stranded amplification products having biotinylated Watson strands with streptavidin-functionalized beads under conditions where the biotinylated Watson strands bind to the streptavidin-functionalized beads; iii) denaturing the double stranded amplification products having biotinylated Watson strands to leave single stranded biotinylated Watson strands bound to the streptavidin-functionalized beads and to release single stranded Crick strands; iv) collecting the single stranded Crick strands; v) releasing the single stranded biotinylated Watson strands from the streptavidin-functionalized beads; and vi) collecting the single stranded biotinylated Watson strands.

In some embodiments, the double stranded DNA template is obtained from a sample from a mammal. In some embodiments, the mammal is a human.

In some embodiments, the method further comprises, prior to the generating a double stranded DNA fragment having the duplex molecular barcode on each end of the double stranded DNA fragment: fragmenting the double stranded DNA to generate double stranded DNA fragments; dephosphorylating 5′ ends of the double stranded DNA fragments; and blunting the ends of double stranded DNA fragments.

In some embodiments, ligating a 3′ duplex adapter to each 3′ end of a double stranded DNA fragment obtained from the double stranded DNA template comprises contacting the 3′ duplex adapter and the double stranded DNA fragment obtained from the double stranded DNA template in the presence of a ligase. In some embodiments, the ligase is a T4 DNA ligase. In some embodiments, the degradable 3′ blocking group comprises contacting the 3′ duplex adapter with uracil-DNA glycosylase (UDG). In some embodiments, ligating the 5′ adapter to each dephosphorylated 5′ end of the double stranded DNA fragment obtained from the double stranded DNA template comprises contacting the 5′ adapter and the double stranded DNA fragment obtained from the double stranded DNA template in the presence of a ligase. In some embodiments, the ligase is an Escherichia coli ligase.

In some embodiments, filling in the gap of single stranded nucleic acid between the 5′ end of the double stranded DNA fragment and the 5′ adapter comprises contacting the 5′ end of the double stranded DNA fragment and the 5′ adapter in the presence of a polymerase and dNTPs. In some embodiments, the polymerase is a Taq-B polymerase. In some embodiments, ligating the 5′ adapter to each 5′ end of the double stranded DNA fragment, and the filling in the gap between the 5′ end of the double stranded DNA fragment and the 5′ adapter are performed concurrently.

In some embodiments, amplifying the double stranded DNA fragment having the duplex molecular barcode on each end of the double stranded DNA fragment comprises contacting the double stranded DNA fragment comprising the duplex molecular barcode on each end of the double stranded DNA fragment with the primer pair under PCR conditions. In some embodiments, amplifying comprises whole-genome PCR. In some embodiments, amplifying the target region from the DNA library (e.g., the single stranded DNA library) of Watson strands further comprises second amplification using a second primer pair consisting of a first primer capable of hybridizing to the target region and a second primer capable of hybridizing to the 3′ duplex adapter; and wherein the amplifying the target region from the DNA library (e.g., the single stranded DNA library) of Crick strands further comprises a second amplification using a second primer pair consisting of a first primer capable of hybridizing to the target region and a second primer capable of hybridizing to the 5′ adapter. In some embodiments, the sequencing steps comprise paired-end sequencing or single-end sequencing.

Also provided herein are methods for detecting the presence or absence of a mutation in a target region of a double stranded DNA template obtained from a sample from a mammal, and determining whether the mutation is present on both strands of the double stranded DNA template, wherein the method comprises: A) generating double stranded DNA fragments each having a duplex molecular barcode on each end of the double stranded DNA fragment; B) amplifying the double stranded DNA fragments each having the duplex molecular barcode on each end of the double stranded DNA fragment using a universal primer pair, wherein the amplifying comprises contacting the double stranded DNA fragment comprising the duplex molecular barcode on each end of the double stranded DNA fragment with the primer pair under whole-genome PCR conditions; C) amplifying the target region from a Watson strand of the amplified double stranded DNA fragments each having the duplex molecular barcode on each end of the double stranded DNA fragment using a primer pair consisting of a first primer capable of hybridizing to the target region and a second primer capable of hybridizing to the 3′ duplex adapter; D) amplifying the target region from a Crick strand of the amplified double stranded DNA fragments each having the duplex molecular barcode on each end of the double stranded DNA fragment using a primer pair consisting of a first primer capable of hybridizing to the target region and a second primer capable of hybridizing to the 5′ adapter; E) sequencing the target region amplified from the Watson strand to generate a sequencing read and to detect the presence or absence of the mutation in a Watson strand of the target region; F) sequencing the target region amplified from the Crick strand to generate a sequencing read and detect the presence or absence of the mutation in a Crick strand of the target region; G) grouping the sequencing reads by the molecular barcode present in each sequencing read to determining whether the mutation is present on both strands of the double stranded DNA template.

In some embodiments, the double stranded DNA template is a genomic DNA sample and generating the double stranded DNA fragments each having the duplex molecular barcode on each end of the double stranded DNA fragment comprises: i) ligating a 3′ duplex adapter to each 3′ end of a double stranded DNA fragment obtained from the double stranded DNA template, wherein the 3′ duplex adapter comprises a) a first oligonucleotide comprising a 5′ phosphate, a first molecular barcode, and a 3′ oligonucleotide annealed to b) a second oligonucleotide comprising a degradable 3′ blocking group, wherein the 3′ oligonucleotide and the second oligonucleotide sequence are complementary; ii) degrading the degradable 3′ blocking group; iii) ligating a 5′ adapter to each dephosphorylated 5′ end of the double stranded DNA fragment obtained from the double stranded DNA template, wherein the 5′ duplex adapter comprises an oligonucleotide comprising a second molecule barcode, wherein the second molecular barcode is different from the first molecular barcode, wherein the 5′ adapter is ligated onto the double stranded DNA fragment upstream of the first molecular barcode and leaving a gap of single stranded nucleic acid between the 5′ end of the double stranded DNA fragment and the 5′ adapter; and iv) filling in the gap of single stranded nucleic acid between the 5′ end of the double stranded DNA fragment and the 5′ adapter to generate the double stranded DNA fragment comprising the duplex molecular barcode on each end of the double stranded DNA fragment. In some embodiments, the double stranded DNA template is a cell-free DNA sample. In some embodiments, the double stranded DNA template is a genomic DNA sample. In some embodiments, the mammal is a human.

In some embodiments, the method further comprises, prior to the generating a double stranded DNA fragment having the duplex molecular barcode on each end of the double stranded DNA fragment: fragmenting the double stranded DNA to generate double stranded DNA fragments; dephosphorylating 5′ ends of the double stranded DNA fragments; and blunting the ends of double stranded DNA fragments.

In some embodiments, ligating a 3′ duplex adapter to each 3′ end of a double stranded DNA fragment obtained from the double stranded DNA template comprises contacting the 3′ duplex adapter and the double stranded DNA fragment obtained from the double stranded DNA template in the presence of a ligase. In some embodiments, the ligase is a T4 DNA ligase. In some embodiments, degrading the degradable 3′ blocking group comprises contacting the 3′ duplex adapter with uracil-DNA glycosylase (UDG). In some embodiments, ligating the 5′ adapter to each dephosphorylated 5′ end of the double stranded DNA fragment obtained from the double stranded DNA template comprises contacting the 5′ adapter and the double stranded DNA fragment obtained from the double stranded DNA template in the presence of a ligase. In some embodiments, the ligase is an Escherichia coli ligase.

In some embodiments, filling in the gap of single stranded nucleic acid between the 5′ end of the double stranded DNA fragment and the 5′ adapter comprises contacting the 5′ end of the double stranded DNA fragment and the 5′ adapter in the presence of a DNA polymerase and dNTPs. In some embodiments, the DNA polymerase is a Taq-B polymerase.

In some embodiments, ligating the 5′ adapter to each 5′ end of the double stranded DNA fragment, and the filling in the gap between the 5′ end of the double stranded DNA fragment and the 5′ adapter are performed concurrently. In some embodiments, amplifying the double stranded DNA fragment having the duplex molecular barcode on each end of the double stranded DNA fragment comprises contacting the double stranded DNA fragment comprising the duplex molecular barcode on each end of the double stranded DNA fragment with the primer pair under PCR conditions. In some embodiments, amplifying comprises whole-genome PCR. In some embodiments, amplifying the target region from the DNA library (e.g., the single stranded DNA library) of Watson strands further comprises second amplification using a second primer pair consisting of a first primer capable of hybridizing to the target region and a second primer capable of hybridizing to the 3′ duplex adapter; and wherein the amplifying the target region from the DNA library (e.g., the single stranded DNA library) of Crick strands further comprises a second amplification using a second primer pair consisting of a first primer capable of hybridizing to the target region and a second primer capable of hybridizing to the 5′ adapter. In some embodiments, the sequencing steps comprise paired-end sequencing.

Also provided herein are methods, comprising:
a. attaching partially double-stranded 3′ adaptors to 3′ ends of both Watson and Crick strands of a population of double-stranded DNA fragments in an analyte DNA sample, wherein a first strand of the partially double-stranded 3′ adaptor comprises, in the 5′-3′ direction, (i) a first segment, (ii) an exogenous UID sequence, (iii) an annealing site for a 5′ adaptor, and (iv) a universal 3′ adaptor sequence comprising an R2 sequencing primer site, and wherein the second strand of the partially double-stranded 3′ adaptor comprises, in the 5′ to 3′ direction, (i) a segment complementary to the first segment, and (ii) a 3′ blocking group, optionally wherein the second strand is degradable;
b. annealing 5′ adaptors to the 3′ adaptors via the annealing site, wherein the 5′ adaptors comprise, in the 5′ to 3′ direction, (i) a universal 5′ adaptor sequence that is not complementary to the universal 3′ adaptor sequence and that comprises an R1 sequencing primer site, and (ii) a sequence complementary to the annealing site for the 5′ adaptor;
c. performing a nick translation-like reaction to extend the 5′ adaptors across the exogenous UID sequence of the 3′ adaptors (e.g., using a DNA polymerase) and covalently link the extended 5′ adaptor to the 5′ ends of the Watson and Crick strands of the double-stranded DNA fragments (e.g., using a ligase);
d. performing an initial amplification to amplify the adaptor-ligated, double-stranded DNA fragments to produce amplicons;
e. determining sequence reads of one or more amplicons of the one or more of the adaptor-ligated, double-stranded DNA fragments;
f. assigning the sequence reads into UID families, wherein each member of a UID family comprises the same exogenous UID sequence;
g. assigning sequence reads of each UID family into a Watson subfamily and Crick subfamily based on spatial relationship of the exogenous UID sequence to the R1 and R2 read sequence;
h. identifying a nucleotide sequence as accurately representing a Watson strand of an analyte DNA fragment when a threshold percentage of members of the Watson subfamily contain the sequence;
i. identifying a nucleotide sequence as accurately representing a Crick strand of an analyte DNA fragment when a threshold percentage of members of the Crick subfamily contain the sequence;
j. identifying a mutation in the nucleotide sequence accurately representing the Watson Strand when the sequence accurately representing the Watson Strand differs from a reference sequence that lacks the mutation;
k. identifying a mutation in the nucleotide sequence accurately representing the Crick Strand when the sequence accurately representing the Crick Strand differs from a reference sequence that lacks the mutation; and
l. identifying a mutation in the analyte DNA fragment when the mutation in the nucleotide sequence accurately representing the Watson strand and the mutation in the nucleotide sequence accurately representing the Crick strand are the same mutation.

In some embodiments, each member of a UID family further comprises the same endogenous UID sequence, wherein the endogenous UID sequence comprises an end of a double-stranded DNA fragment from the population. In some embodiments, the endogenous UID sequence comprising the end of the double-stranded DNA fragment comprises at least 8, 10, or 15 bases. In some embodiments, exogenous UID sequence is unique to each double-stranded DNA fragment. In some embodiments, the exogenous UID sequence is not unique to each double-stranded DNA fragment. In some embodiments, each member of a UID family comprises the same endogenous UID sequence and the same exogenous UID sequence. In some embodiments, step (d) comprises no more than 11 cycles of PCR amplification. In some embodiments, step (d) comprises no more than 7 cycles of PCR amplification. In some embodiments, step (d) comprises no more than 5 cycles of PCR amplification. In some embodiments, step (d) comprises at least 1 cycles of PCR amplification.

In some embodiments, prior to determining the sequence reads, the amplicons are enriched for one or more target polynucleotides. In some embodiments, the enriching comprises:
a. selectively amplifying amplicons of Watson strands comprising the target polynucleotide sequence with a first set of Watson target-selective primer pairs, the first set of Watson target-selective primer pairs comprising: (i) a first Watson target-selective primer comprising a sequence complementary to a portion of the universal 3′ adapter sequence, optionally wherein the portion of the universal 3′ adapter sequence is the R2 sequencing primer site of the universal 3′ adaptor sequence, and (ii) a second Watson target-selective primer comprising a target-selective sequence, thereby creating target Watson amplification products; and
b. selectively amplifying amplicons of Crick strands comprising the same target polynucleotide sequence with a first set of Crick target-selective primer pairs, the first set of Crick target-selective primer pairs comprising: (i) a first Crick target-selective primer comprising a sequence complementary to a portion of the universal 5′ adapter sequence, optionally wherein the portion of the universal 5′ adapter sequence is the R1 sequencing primer site of the universal 5′ adaptor sequence, and (ii) a second Crick target-selective primer comprising the same target-selective sequence as the second Watson target-selective primer sequence, thereby creating target Crick amplification products.

In some embodiments, the method further comprises purifying the target Watson amplification products and the target Crick amplification products from non-target polynucleotides. In some embodiments, the method further comprises purifying comprises attaching the target Watson amplification products and the target Crick amplification products to a solid support. In some embodiments, the first Watson target-selective primer and first Crick target-selective primer comprises a first member of an affinity binding pair, and wherein the solid support comprises a second member of the affinity binding pair. In some embodiments, the first member is biotin and the second member is streptavidin. In some embodiments, the solid support comprises a bead, well, membrane, tube, column, plate, sepharose, magnetic bead, or chip. In some embodiments, the method further comprises removing polynucleotides that are not attached to the solid support.

In some embodiments, the method further comprises:
a. further amplifying the target Watson amplification products with a second set of Watson target-selective primers, the second set of Watson target-selective primers comprising (i) a third Watson target-selective primer comprising a sequence complementary to a portion of the universal 3′ adapter sequence, optionally wherein the portion of the universal 3′ adapter sequence is the R2 sequencing primer site of the universal 3′ adaptor sequence, and (ii) a fourth Watson target-selective primer comprising, in the 5′ to 3′ direction, an R1 sequencing primer site and a target-selective sequence selective for the same target polynucleotide, thereby creating target Watson library members;
b. further amplifying the target Crick amplification products with a second set of Crick target-selective primers, the second set of Crick target-selective primers comprising (i) a third Crick target-selective primer comprising a sequence complementary to a portion of the universal 5′ adapter sequence, optionally wherein the portion of the universal 5′ adapter sequence is the R1 sequencing primer site of the universal 5′ adaptor sequence, and (ii) a fourth Crick target-selective primer comprising, in the 5′ to 3′ direction, an R2 sequencing primer site and the target-selective sequence selective for the same target polynucleotide of the fourth Watson target-selective primer, thereby creating target Crick library members.

In some embodiments, the third Watson and Crick target-selective primers further comprise a sample barcode sequence. In some embodiments, the third Watson target-selective primer further comprises a first grafting sequence that enables hybridization to a first grafting primer on a sequencer and wherein the third Crick target-selective primer further comprises a second grafting sequence that enables hybridization to a second grafting primer on the sequencer. In some embodiments, the fourth Watson target-selective primer further comprises the second grafting sequence and wherein the fourth Crick target-selective primer further comprises the first grafting sequence. In some embodiments, the first grafting sequence is a P7 sequence and wherein the second grafting sequence is a P5 sequence. In some embodiments, the target Watson library members and the target Crick library members represent at least 50% of the target polynucleotides in the population of double-stranded DNA fragments. In some embodiments, the target Watson library members and the target Crick library members represent at least 70% of the target polynucleotides in the population of double-stranded DNA fragments. In some embodiments, the target Watson library members and the target Crick library members represent at least 80% of the target polynucleotides in the population of double-stranded DNA fragments. In some embodiments, the target Watson library members and the target Crick library members represent at least 90% of the target polynucleotides in the population of double-stranded DNA fragments. In some embodiments, the target Watson library members and the target Crick library members represent at least 50% of the population of total DNA fragments. IN some embodiments, the target Watson library members and the target Crick library members represent at least 70% of the population of total DNA fragments. In some embodiments, the target Watson library members and the target Crick library members represent at least 80% of the population of total DNA fragments. In some embodiments, the target Watson library members and the target Crick library members represent at least 90% of the population of total DNA fragments.

Also provided herein are methods comprising:
a. attaching adaptors to a population of double-stranded DNA fragments in an analyte DNA sample, wherein the adaptors comprise a double-stranded portion comprising an exogenous UID and a forked portion comprising (i) a single-stranded 3′ adaptor sequence comprising an R2 sequencing primer site and (ii) a single-stranded 5′ adaptor sequence comprising an R1 sequencing primer site;
b. performing an initial amplification to amplify the adaptor-ligated, double-stranded DNA fragments to produce amplicons;
c. selectively amplifying amplicons of Watson strands comprising a target polynucleotide sequence with a first set of Watson target-selective primer pairs, the first set of Watson target-selective primer pairs comprising: (i) a first Watson target-selective primer comprising a sequence complementary to a portion of the universal 3′ adapter sequence, optionally wherein the portion of the universal 3′ adapter sequence is the R2 sequencing primer site of the universal 3′ adaptor sequence, and (ii) a second Watson target-selective primer comprising a target-selective sequence, thereby creating target Watson amplification products;
d. selectively amplifying amplicons of Crick strands comprising the same target polynucleotide sequence with a first set of Crick target-selective primer pairs, the first set of Crick target-selective primer pairs comprising: a first Crick target-selective primer comprising a sequence a sequence complementary to a portion of the universal 5′ adapter sequence, optionally wherein the portion of the universal 5′ adapter sequence is the R1 sequencing primer site of the universal 5′ adaptor sequence, and (ii) a second Crick target-selective primer comprising the same target-selective sequence as the second Crick target-selective primer sequence, thereby creating target Crick amplification products.
e. determining sequence reads of the target Watson amplification products and the target Crick amplification products;
f. assigning the sequence reads into UID families, wherein each member of a UID family comprises the same exogenous UID sequence;
g. assigning sequence reads of each UID family into a Watson subfamily and Crick subfamily based on spatial relationship of the exogenous UID sequence to the R1 and R2 read sequence;
h. identifying a nucleotide sequence as accurately representing a Watson strand of an analyte DNA fragment when a threshold percentage of members of the Watson family contain the sequence;
i. identifying a nucleotide sequence as accurately representing a Crick strand of an analyte DNA fragment when a threshold percentage of members of the Crick family contain the sequence; and
j. identifying a mutation in the analyte DNA fragment when the nucleotide sequence accurately representing the Watson strand and the nucleotide sequence accurately representing the Crick strand both contain the same mutation.

In some embodiments, the method further comprises purifying the target Watson amplification products and the target Crick amplification products from non-target polynucleotides. In some embodiments, the method further comprises attaching the target Watson amplification products and the target Crick amplification products to a solid support. In some embodiments, the first Watson target-selective primer and first Crick target-selective primer comprises a first member of an affinity binding pair, and wherein the solid support comprises a second member of the affinity binding pair. In some embodiments, the first member is biotin and the second member is streptavidin. In some embodiments, the solid support comprises a bead, well, membrane, tube, column, plate, sepharose, magnetic bead, or chip. In some embodiments, the method further comprises removing polynucleotides that are not attached to the solid support.

In some embodiments, the method further comprises:
a. further amplifying the target Watson amplification products with a second set of Watson target-selective primers, the second set of Watson target-selective primers comprising (i) a third Watson target-selective primer comprising a sequence complementary to the R2 sequencing primer site of the universal 3′ adaptor sequence, and (ii) a fourth Watson target-selective primer comprising, in the 5′ to 3′ direction, an R1 sequencing primer site and a target-selective sequence selective for the same target polynucleotide, thereby creating target Watson library members;
b. further amplifying the target Crick amplification products with a second set of Crick target-selective primers, the second set of Crick target-selective primers comprising (i) a third Crick target-selective primer comprising a sequence complementary to the R1 sequencing primer site of the universal 3′ adaptor sequence, and (ii) a fourth Crick target-selective primer comprising, in the 5′ to 3′ direction, an R2 sequencing primer site and the target-selective sequence selective for the same target polynucleotide of the fourth Watson target-selective primer, thereby creating target Crick library members, thereby creating target Crick library members.

In some embodiments, the third Watson and Crick target-selective primers further comprise a sample barcode sequence. In some embodiments, the third Watson target-selective primer further comprises a first grafting sequence that enables hybridization to a first grafting primer on a sequencer and wherein the third Crick target-selective primer further comprises a second grafting sequence that enables hybridization to a second grafting primer on the sequencer. In some embodiments, the fourth Watson target-selective primer further comprises the second grafting sequence and wherein the fourth Crick target-selective primer further comprises the first grafting sequence. In some embodiments, the first grafting sequence is a P7 sequence and wherein the second grafting sequence is a P5 sequence. In some embodiments, the attaching comprises attaching A-tailed adaptors to the population of double-stranded DNA fragments. In some embodiments, the attaching comprises attaching A-tailed adaptors to both ends of DNA fragments in the population.

In some embodiments, the attaching comprises:
a. attaching partially double-stranded 3′ adaptors to 3′ ends of both Watson and Crick strands of a population of double-stranded DNA fragments, wherein a first strand of the partially double-stranded 3′ adaptor comprises, in the 5′-3′ direction, (i) a first segment, (ii) optionally, an exogenous UID sequence, (iii) an annealing site for a 5′ adaptor, and (iv) a universal 3′ adaptor sequence comprising an R2 sequencing primer site, and wherein the second strand of the partially double-stranded 3′ adaptor comprises, in the 5′ to 3′ direction, (i) a segment complementary to the first segment, and (ii) a 3′ blocking group, optionally wherein the second strand is degradable; and
b. annealing 5′ adaptors to the 3′ adaptors via the annealing site, wherein the 5′ adaptors comprise, in the 5′ to 3′ direction, (i) a universal 5′ adaptor sequence that is not complementary to the universal 3′ adaptor sequence and that comprises an R1 sequencing primer site, and (ii) a sequence complementary to the annealing site for the 5′ adaptor; and
c. performing a nick translation-like reaction to extend the 5′ adaptors across the 3′ adaptors (e.g., using a DNA polymerase) and covalently link the extended 5′ adaptor to the 5′ ends of the Watson and Crick strands of the double-stranded DNA fragments (e.g. using a ligase).

In some embodiments, the UID sequence comprises an endogenous UID sequence comprising an end of a double-stranded DNA fragment from the population. In some embodiments, the endogenous UID sequence comprising the end of the double-stranded DNA fragment comprises at least 8, 10, or 15 bases. In some embodiments, the exogenous UID sequence is unique to each double-stranded DNA fragment. In some embodiments, the exogenous UID sequence is not unique to each double-stranded DNA fragment. In some embodiments, each member of a UID family comprises the same endogenous UID sequence and the same exogenous UID sequence.

In some embodiments, amplifying the adaptor-ligated, double-stranded DNA fragments to produce amplicons comprises no more than 11 cycles of PCR amplification. In some embodiments, amplifying the adaptor-ligated, double-stranded DNA fragments to produce amplicons comprises no more than 7 cycles of PCR amplification. In some embodiments, amplifying the adaptor-ligated, double-stranded DNA fragments to produce amplicons comprises no more than 5 cycles of PCR amplification. In some embodiments, amplifying the adaptor-ligated, double-stranded DNA fragments to produce amplicons comprises at least 1 cycles of PCR amplification. In some embodiments, the target Watson library members and the target Crick library members represent at least 50% of the target polynucleotides in the population of double-stranded DNA fragments. In some embodiments, the target Watson library members and the target Crick library members represent at least 70% of the target polynucleotides in the population of double-stranded DNA fragments. In some embodiments, the target Watson library members and the target Crick library members represent at least 80% of the target polynucleotides in the population of double-stranded DNA fragments. In some embodiments, the target Watson library members and the target Crick library members represent at least 90% of the target polynucleotides in the population of double-stranded DNA fragments. In some embodiments, the target Watson library members and the target Crick library members represent at least 50% of the population of total DNA fragments. In some embodiments, the target Watson library members and the target Crick library members represent at least 70% of the population of total DNA fragments. In some embodiments, the target Watson library members and the target Crick library members represent at least 80% of the population of total DNA fragments. In some embodiments, the target Watson library members and the target Crick library members represent at least 90% of the population of total DNA fragments.

In some embodiments, the determination of the sequence reads enables sequence determination of both ends of template molecules. In some embodiments, the determination of both ends of template molecules comprises paired end sequencing. In some embodiments, the determination of the sequence reads comprises single read sequencing across the length of the templates for generating the sequence reads. In some embodiments, the determination of the sequence reads comprises sequencing with a massively parallel sequencer. In some embodiments, the massively parallel sequencer is configured to determine sequence reads from both ends of template polynucleotides. In some embodiments, the population of double-stranded DNA fragments comprises one or more fragments that are about 50-600 nt in length. In some embodiments, the population of double-stranded DNA fragments comprises one or more fragments that are less than 2000, less than 1000, less than 500, less than 400, less than 300, or less than 250 nt in length.

In some embodiments, methods provided herein further comprise, after the initial amplification and before the selective amplification, preparing single stranded (ss) DNA libraries corresponding to sense and antisense strands of the amplicons. In some embodiments, the ss DNA library preparation comprises:
a. performing an amplification reaction utilizing two primers, wherein only one of the two primers comprises a first member of an affinity binding pair, thereby creating amplification products comprising a strand comprising the first member of the affinity binding pair and a strand not comprising the first member of the affinity binding pair;
b. contacting the amplification products with a solid support, wherein the solid support comprises the second member of the affinity binding pair;
c. denaturing the amplification products to separate the strand comprising the first member of the affinity binding pair from the strand not comprising the first member of the affinity binding pair; and
d. purifying the separated strands comprising the first member of the affinity binding pair and the separated strands not comprising the first member of the affinity binding pair.

In some embodiments, the first member of the affinity binding pair is biotin and the second member of the affinity binding pair is streptavidin. In some embodiments, the ss DNA library preparation comprises:
a. partitioning the amplicons into two amplification reactions, each amplification reaction utilizing a forward primer and a reverse primer, wherein only one of the two primers is phosphorylated, thereby creating amplification products comprising a phosphorylated strand and a non-phosphorylated strand;
b. contacting the amplification products with an exonuclease that selectively digests the strands with the 5′ phosphate.

In some embodiments:
a. in the first amplification reaction, the forward primer is phosphorylated and the reverse primer is non-phosphorylated;
b. in the second amplification reaction, the reverse primer is phosphorylated and the forward primer is non-phosphorylated.

In some embodiments, the exonuclease is a lambda exonuclease. In some embodiments, the phosphorylation is at the 5′ site.

In some embodiments, the initial amplification comprises:
a. amplifying with a primer pair, in which only one of the two primers in the primer pair comprises a first member of an affinity binding pair, thereby creating amplification products comprising a strand comprising the first member of the affinity binding pair and a strand not comprising the first member of the affinity binding pair;
b. contacting the amplification products with a solid support, wherein the solid support comprises the second member of the affinity binding pair;
c. denaturing the amplification products to separate the strand comprising the first member of the affinity binding pair from the strand not comprising the first member of the affinity binding pair; and
d. purifying the separated strands comprising the first member of the affinity binding pair and the separated strands not comprising the first member of the affinity binding pair.

In some embodiments, the first member of the affinity binding pair is biotin and the second member of the affinity binding pair is streptavidin. In some embodiments, sequence reads of a UID family are assigned to a Watson subfamily when the exogenous UID sequence is downstream of the R2 sequence and upstream of the R1 sequence. In some embodiments, sequence reads of a UID family are assigned to a Crick subfamily when the exogenous UID sequence is downstream of the R1 sequence and upstream of the R2 sequence. In some embodiments, sequence reads of a UID family are assigned to a Watson subfamily when the exogenous UID sequence is in greater proximity to the R2 sequence and lesser proximity to the R1 sequence. In some embodiments, sequence reads of a UID family are assigned to a Crick subfamily when the exogenous UID sequence is in greater proximity to the R1 sequence and in lesser proximity to the R2 sequence. In some embodiments, sequence reads of a UID family are assigned to a Watson subfamily when the exogenous UID sequence is immediately downstream or within 1-300, 1-70, 1-60, 1-50, 1-40, 1-30, 1-20, 1-10, or 1-5 nucleotides of the R2 sequence. In some embodiments, sequence reads of a UID family are assigned to a Crick subfamily when the exogenous UID sequence is immediately downstream or within 1-300, 1-70, 1-60, 1-50, 1-40, 1-30, 1-20, 1-10, or 1-5 nucleotides of the R1 sequence.

In some embodiments, the population of double-stranded DNA fragments are from a biological sample. In some embodiments, the biological sample is obtained from a subject. In some embodiments, the subject is a human subject. In some embodiments, the biological sample is a fluid sample. In some embodiments, the fluid sample is selected from whole blood, plasma, serum sputum, urine, sweat, tears, ascites, semen, and bronchoaveolar lavage. In some embodiments, the fluid sample is a cell-free or essentially cell-free sample. In some embodiments, the biological sample is a solid biological sample. In some embodiments, the solid biological sample is a tumor sample.

In some embodiments, the identified mutation is present in the population of double-stranded DNA fragments at a frequency of 0.1% or less. In some embodiments, the identified mutation is present in the population of double-stranded DNA fragments at a frequency of 0.1% to 0.00001%. In some embodiments, the identified mutation is present in the population of double-stranded DNA fragments at a frequency of 0.1% to 0.01%. In some embodiments, the determining of sequence reads comprises determining sequence reads from both Watson and Crick strands of at least 50% of the double-stranded DNA fragments comprising a target polynucleotide in the analyte DNA sample. In some embodiments, the determining of sequence reads comprises determining sequence reads from both Watson and Crick strands of at least 70% of the double-stranded DNA fragments comprising a target polynucleotide in the analyte DNA sample. In some embodiments, the determining of sequence reads comprises determining sequence reads from both Watson and Crick strands of at least 80% of the double-stranded DNA fragments comprising a target polynucleotide in the analyte DNA sample. In some embodiments, the determining of sequence reads comprises determining sequence reads from both Watson and Crick strands of at least 90% of the double-stranded DNA fragments comprising a target polynucleotide in the analyte DNA sample. In some embodiments, the determining of sequence reads comprises determining sequence reads from both Watson and Crick strands of at least 50% of the double-stranded DNA fragments in the analyte DNA sample. In some embodiments, the determining of sequence reads comprises determining sequence reads from both Watson and Crick strands of at least 70% of the double-stranded DNA fragments in the analyte DNA sample. In some embodiments, the determining of sequence reads comprises determining sequence reads from both Watson and Crick strands of at least 80% of the double-stranded DNA fragments in the analyte DNA sample. In some embodiments, the determining of sequence reads comprises determining sequence reads from both Watson and Crick strands of at least 90% of the double-stranded DNA fragments in the analyte DNA sample.

In some embodiments, the error rate associated with the identification of one or more mutations in analyte DNA fragments according to a method of any one of the preceding claims is reduced by at least 2-fold, 4-fold, 5-fold, 10-fold, 20-fold, 30-fold, 40-fold, 50-fold, 60-fold, 70-fold, 80-fold, 90-fold, or 100-fold, as compared to an alternative method of identifying mutations that does not require the mutation to be detected in both Watson and Crick strands of an analyte DNA fragment. In some embodiments, the alternative method comprises standard molecular barcoding or standard PCR-based molecular barcoding. In some embodiments, the alternative method comprises:
a. attaching adaptors to a population of double-stranded DNA fragments in an analyte DNA sample, wherein the adaptors comprise a unique exogenous UID;
b. performing an initial amplification to amplify the adaptor-ligated, double-stranded DNA fragments to produce amplicons;
c. determining sequence reads of one or more amplicons of the one or more of the adaptor-ligated, double-stranded DNA fragments;
d. assigning the sequence reads into UID families, wherein each member of a UID family comprises the same exogenous UID sequence;
e. identifying a nucleotide sequence as accurately representing an analyte DNA fragment when a threshold percentage of members of a UID family contain the sequence; and
f. identifying a mutation in the analyte DNA fragment when the sequence identified as accurately representing an analyte DNA fragment differs from a reference sequence that lacks the mutation.

In some embodiments, the error rate associated with the identification of one or more mutations in analyte DNA fragments according to a method of any one of the preceding claims is no more than 1×10⁻², no more than 1×10⁻³, no more than 1×10⁻⁴, no more than 1×10⁻⁵, no more than 1×10⁻⁶, no more than 5×10⁻⁶, or no more than 1×10⁻⁷.

Also provided herein are computer readable media comprising computer executable instructions for analyzing sequence read data from a nucleic acid sample, wherein the data is generated by a method of any one of the preceding claims. In some embodiments, the computer readable medium comprises executable instructions for
a. assigning sequence reads into UID families, wherein each member of a UID family comprises the same exogenous UID sequence;
b. assigning sequence reads of each UID family into Watson and Crick subfamilies based on spatial relationship of the exogenous UID sequence to the R1 and R2 read sequence;
c. identifying a nucleotide sequence as accurately representing a Watson strand of an analyte DNA fragment when a threshold percentage of members of the Watson subfamily contain the sequence;
d. identifying a nucleotide sequence as accurately representing a Crick strand of an analyte DNA fragment when a threshold percentage of members of the Crick subfamily contain the sequence;
e. identifying a mutation in the nucleotide sequence accurately representing the Watson Strand when the sequence accurately representing the Watson Strand differs from a reference sequence that lacks the mutation;
f. identifying a mutation in the nucleotide sequence accurately representing the Crick Strand when the sequence accurately representing the Crick Strand differs from a reference sequence that lacks the mutation;
g. identifying a mutation in the analyte DNA fragment when the mutation in the nucleotide sequence accurately representing the Watson strand and the mutation in the nucleotide sequence accurately representing the Crick strand are the same mutation.

In some embodiments, the computer readable medium comprises executable instructions for assigning UID family members to the Watson subfamily when the exogenous UID sequence is immediately downstream or within 1-300 nucleotides of the R2 sequencing primer binding site. In some embodiments, the computer readable medium comprises executable instructions for assigning UID family members to the Crick subfamily when the exogenous UID sequence is immediately downstream or within 1-300 nucleotides of the R1 sequencing primer binding site. In some embodiments, the computer readable medium comprises executable instructions for mapping the sequence reads to a reference genome. In some embodiments, the reference genome is a human reference genome.

In some embodiments, the computer readable medium further comprises computer executable instructions to generate a report of therapeutic options based on the presence, absence, or amount of a mutation in the sample. In some embodiments, the computer readable medium further comprises computer executable code that enables transmission of the data over a network.

Also provided herein are computer systems, comprising:
a. a memory unit configured to receive sequence data from a nucleic acid sample, wherein the data is generated by a method of any one of the preceding claims;
b. a processor communicatively coupled to the memory unit, wherein the processor comprises a computer readable medium of any of the preceding claims.

In some embodiments, the computer system further comprises a sequencing system configured to communicate the data to the memory unit. In some embodiments, the computer system further comprises a user interface configured to communicate or display the report to a user. In some embodiments, the computer system further comprises a digital processor configured to transmit results of the data analysis over a network.

Also provided herein are systems, comprising:
a. a population of double-stranded DNA fragments from a biological sample;
b. a population of 3′ adaptors as described in any one of the preceding claims;
c. a population of 5′ adaptors as described in any one of the preceding claims;
d. reagents for performing nick translation-like reaction (e.g., with a DNA polymerase, cohesive end-specific ligase, and uracil-DNA glycosylase);
e. reagents for enriching amplicons for one or more target polynucleotides; and
f. a sequencing system.

In some embodiments, the system further comprises a computer system of any one of the preceding claims.

Also provided herein are kits, comprising:
a. a first set of Watson target-selective primer pairs, comprising (i) one or more first Watson target-selective primers comprising a sequence complementary to a portion of the universal 3′ adapter sequence, optionally wherein the portion of the universal 3′ adapter sequence is the R2 sequencing primer site of the universal 3′ adaptor sequence, and (ii) one or more second Watson target-selective primers, each of the one or more second Watson target-selective primers comprising a target-selective sequence;
b. a first set of Crick target-selective primer pairs, comprising (i) one or more Crick target-selective primers comprising a sequence a sequence complementary to a portion of the universal 5′ adapter sequence, optionally wherein the portion of the universal 5′ adapter sequence is the R1 sequencing primer site of the universal 5′ adaptor sequence, and (ii) one or more second Crick target-selective primers, each of the one or more second Crick target-selective primers comprising the same target-selective sequence as the second Watson target-selective primer sequence;
c. a second set of Watson target-selective primer pairs, comprising (i) one or more third Watson target-selective primers comprising a sequence complementary to the R2 sequencing primer site of the universal 3′ adaptor sequence, and (ii) one or more fourth Watson target-selective primers, each of the one or more fourth Watson target-selective primers comprising, in the 5′ to 3′ direction, an R1 sequencing primer site and a target-selective sequence selective for the same target polynucleotide; and
d. a second set of Crick target-selective primers comprising (i) one or more third Crick target-selective primers comprising a sequence complementary to the R1 sequencing primer site of the universal 3′ adaptor sequence, and (ii) one or more fourth Crick target-selective primers, each of the one or more fourth Crick target-selective primers comprising, in the 5′ to 3′ direction, an R2 sequencing primer site and a target-selective sequence selective for the same target polynucleotide.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 contains a schematic of an exemplary duplex anchored PCR method. Duplex adapters with molecular barcodes are ligated onto the ends of nucleic acid fragments having blunted ends, to generate a duplex sequencing library, and the duplex sequencing library is subjected to PCR to generate an amplified duplex sequencing library. The amplification products in the amplified duplex sequencing library are divided into two aliquots and each aliquot is subjected to PCR where the Watson strand is amplified from the first aliquot, and the Crick strand is amplified from the second aliquot.

FIG. 2 contains a schematic of an exemplary second round of library amplification where the Watson strands amplified from the first aliquot in FIG. 1 are subjected to PCR using a primer pair where a first primer is biotinylated and a second primer is non-biotinylated to generate a single stranded DNA library that can be used to amplify and assess Watson strands.

FIG. 3 contains a schematic of an exemplary second round of library amplification where the Crick strands amplified from the first aliquot in FIG. 1 are subjected to PCR using a primer pair where a first primer is non-biotinylated and a second primer is biotinylated to generate a single stranded DNA library that can be used to amplify and assess Crick strands.

FIG. 4 contains a schematic of an exemplary Watson amplification.

FIG. 5 contains a schematic of an exemplary Crick amplification.

FIG. 6 contains a schematic of an exemplary amplified Watson strand and an exemplary amplified Crick strand.

FIG. 7 contains a schematic of an exemplary nested Watson amplification.

FIG. 8 contains a schematic of an exemplary nested Crick amplification.

FIG. 9 contains a schematic of an exemplary removal of a 5′ phosphate.

FIG. 10 contains a schematic of an exemplary filling in of a 3′ end of an amplification fragment having a 5′ overhang to generate a blunt end amplification product.

FIG. 11 contains a schematic of an exemplary 3′ duplex adapter including a 3SpC3 spacer, exogenous UID sequence containing a molecular barcode, and a 3′ oligonucleotide (dT) hybridized to a 3′ blocking group which can be degraded by uracil-DNA glycosylase (UDG).

FIG. 12 contains a schematic of an exemplary 3′ adapter ligation using a 3′ duplex adapter. The 5′ phosphate of the 3′ duplex adapter is ligated to the 3′ end of the nucleic acid template.

FIG. 13 contains a schematic of an exemplary 5′ adapter ligation. In a single reaction, the blocking group of the 3′ duplex adapter is degraded and a 5′ adapter containing is ligated to the 5′ end of the nucleic acid template via a nick-translation reaction.

FIG. 14 contains a schematic of an exemplary library PCR amplification.

FIG. 15 contains a schematic of an exemplary Watson amplification.

FIG. 16 contains a schematic of an exemplary nested Watson amplification.

FIG. 17 contains a schematic of an exemplary Crick amplification.

FIG. 18 contains a schematic of an exemplary nested Crick amplification.

FIG. 19 contains a schematic of final amplification products generated by an exemplary duplex anchored PCR.

FIG. 20 contains a schematic showing how paired-end sequencing can be used to distinguish the Watson strand from the Crick strand of the input nucleic acid using the final amplification products generated by an exemplary duplex anchored PCR.

FIG. 21 contains a schematic showing how paired-end sequencing can be used to distinguish the Watson strand from the Crick strand of the input nucleic acid using the final amplification products generated by an exemplary duplex anchored PCR.

FIG. 22 contains a schematic of an overview of an exemplary SaferSeqS method. (a) Library preparation begins with end repair (step 1) in which DNA template molecules are dephosphorylated and blunted. Next, a 3′ adapter containing a unique identifier (UID) sequence (narrow or wide diagonal cross-hatching) is ligated to the 3′ fragment ends (step 2). The UID sequences are converted into double stranded barcodes upon extension and ligation of the 5′ adapter (step 3). Finally, redundant PCR copies of each original template molecule are generated during library amplification (step 4). (b) Target enrichment is achieved with strand-specific hemi-nested PCRs. The amplified library is partitioned into Watson and Crick-specific reactions (step 5) which selectively amplify products derived from one of the DNA strands (step 6). Additional on-target specificity and incorporation of sample barcodes are achieved with a second nested PCR (step 7). The final PCR products (step 8) are subjected to paired-end sequencing (step 9). The endogenous barcode represents the end of the template fragment prior to library construction. (c) Following sequencing, reads are determined to be derived from the Watson or Crick strand. Because each strand of the original template molecules is tagged with the same exogenous barcode, and has the same endogenous barcode, reads derived from each of the two strands of the same parental DNA duplex can be grouped together into a duplex family. The different cross-hatch and stipple patterns at the right ends of the strands represent different barcodes. In the examples shown, each duplex family has eight members, four representing the Watson strand and four representing the Crick strand. In the actual experiments described in this paper, each family contains at least two members from the Watson strand and two from the Crick strand, with the actual number dependent on the depth of sequencing. Bonafide mutations, represented by the asterisks within the Bona Fide Mutation family, are present in both parental strands of a DNA duplex and are therefore found in both Watson and Crick families. In contrast, PCR or sequencing errors, represented by asterisks within the PCR of Sequencing Error family, are limited to a subset of reads from one of the two strands. Watson strand-specific (asterisks within Damaged Watson Strand family) and Crick strand-specific (asterisks within the Damaged Crick Strand family) artifacts are found in all copies of the Watson or Crick family, but not in both.

FIG. 23 contains a graph shown an analytical performance of SaferSeqS. Mutant allele frequencies (MAF) determined by SaferSeqS versus the expected frequencies when DNA from a cancer containing a known mutation was mixed with leukocyte DNA from a healthy donor at ratios varying from 10% down to 0.001%. A 0% control sample was also assayed to determine specificity for the mutation of interest. The solid line represents a fit of a linear regression model in which the y-intercept was fixed at zero (slope=0.776, R²>0.999, P=3.95×10⁻¹⁵).

FIG. 24. High duplex recovery and efficient target enrichment with SaferSeqS. Thirty-three ng of admixed cfDNA samples were assayed for one of three different mutations in TP53 (p.L264fs, p.P190L, or p.R342X). Three libraries were prepared per cfDNA sample, each containing ˜11 ng of cfDNA. (a) The median number of duplex families (i.e., both Watson and Crick strands containing the same endogenous and exogenous barcodes) was 89% (range: 65% to 102%) of the number of original template molecules. (b) The median fraction of on-target reads was 80% (range: 72% to 91%). Lower and upper hinges correspond to the 25^{th} and 75^{th} percentile, whiskers extend to 1.5 times the interquartile range. Individual points are overlaid with random scatter for ease of visualization.

FIG. 25 contains graphs showing the detection of exemplary mutations in liquid biopsy samples. Analysis of 33 ng of plasma cell-free DNA from healthy individuals admixed with cell-free plasma DNA from a cancer patient. The mixtures were created to generate a high frequency (˜0.5-1%) of mutation, low frequency (˜0.01-0.1%) of mutation, or no mutation. The admixed TP53p.R342X sample was assayed with (a) SafeSeqs or (b) SaferSeqS. Similarly, the admixed TP53p.L264fs sample was assayed with (c) SafeSeqs and (d) SaferSeqS, and the admixed TP53p.P190L sample was assayed with (e) SafeSeqs and (f) SaferSeqS. Mutation numbers represent each of the 153 distinct mutations observed with SafeSeqS (defined in Table 8). The single supercalimutant detected by SaferSeqS (Table 9) was outside the genomic region assayed by SafeSeqS and is therefore not shown.

FIG. 26. Errors in SaferSeqS as compared to those of strand-agnostic, ligation-based molecular barcoding methods. Analysis of 33 ng of plasma cell-free DNA from healthy individuals admixed with cell-free plasma DNA from a cancer patient. The mixtures were created to generate a high frequency (˜0.5-1%) of mutation, low frequency (˜0.01-0.1%) of mutation, or no mutation. The admixed TP53 p.R342X sample was assayed with SaferSeqS but (a) strand information was ignored in the analysis to mimic strand-agnostic, ligation-based molecular barcoding methods or (b) strand information was considered during mutation calling. Similarly, the admixed TP53 p.L264fs sample was assayed (c) without consideration of strand information and (d) with SaferSeqS. The admixed TP53 p.P190L sample was similarly assayed (e) without consideration of strand information and (f) with SaferSeqS. Mutation numbers are defined in Supplementary Table 3. The asterisks denote the admixed mutations. The single unexpected supercalimutant detected by SaferSeqS is shown in (e).

FIG. 27. Evaluation of plasma samples from cancer patients. Plasma cell-free DNA samples from five cancer patients harboring eight known mutations at frequencies between 0.01% and 0.1% were assayed with a previously described, PCR-based molecular barcoding method (“SafeSeqS” rather than “SaferSeqS”) and with SaferSeqS. Mutation numbers are defined in Table 11. Asterisks denote the expected mutations. The single unexpected supercalimutant detected by SaferSeqS (Table 11) was outside the genomic region assayed by SafeSeqS and is therefore not shown.

FIG. 28. Effects of PCR efficiency and cycle number on duplex recovery. The probability of recovering both strands of the original DNA duplexes (y-axis) is plotted against library amplification cycle number (x-axis). Each pane in the figure represents the assumed PCR efficiency denoted at the top of the pane. The proportion of the library amplification product used in the strand-specific PCRs are shown. Library amplification cycle number was varied from one to 11. PCR efficiency was varied from 100% to 50% in 10% increments. The proportion of library amplification product using in each strand-specific PCRs was varied from 50% to 1.4%. Probabilistic modeling was performed as described in Example 2.

FIG. 29 contains graphs showing a multiplex panel for detection of exemplary cancer driver gene mutations. Recovery and coverage of the 36 amplicons that successfully amplified within the multiplex panel. The horizontal axis displays the position downstream of the 3′ end of the second gene-specific primer (GSP2). The gradual decline in coverage with increasing distance from the 3′ primer end is a consequence of the input DNA fragmentation pattern. Details regarding the theoretical recovery of reads with specific amplicon lengths are discussed in Example 2.

FIG. 30. Performance of the 48 primer pairs used in a multiplex panel to assay regions of driver genes commonly mutated in cancer. The proportion of on-target reads (i.e. the fraction of total reads that map to the intended target) for each of the 48 SaferSeqS primer pairs used in the strand-specific PCRs. Primers were used at equimolar concentrations in each gene-specific PCR.

FIG. 31. Performance of 62 primer pairs. The proportion of on-target reads (i.e. the fraction of total reads that map to the intended target) for each of 62 SaferSeqS primer pairs tested to date. Fifty of the 62 pairs (81%) exhibit an on-target rate of greater than 50%. The results presented reflect a single attempt at primer design.

FIG. 32 depicts an exemplary computer system adapted to enable a user to analyze a nucleic acid sample according to a method described herein.

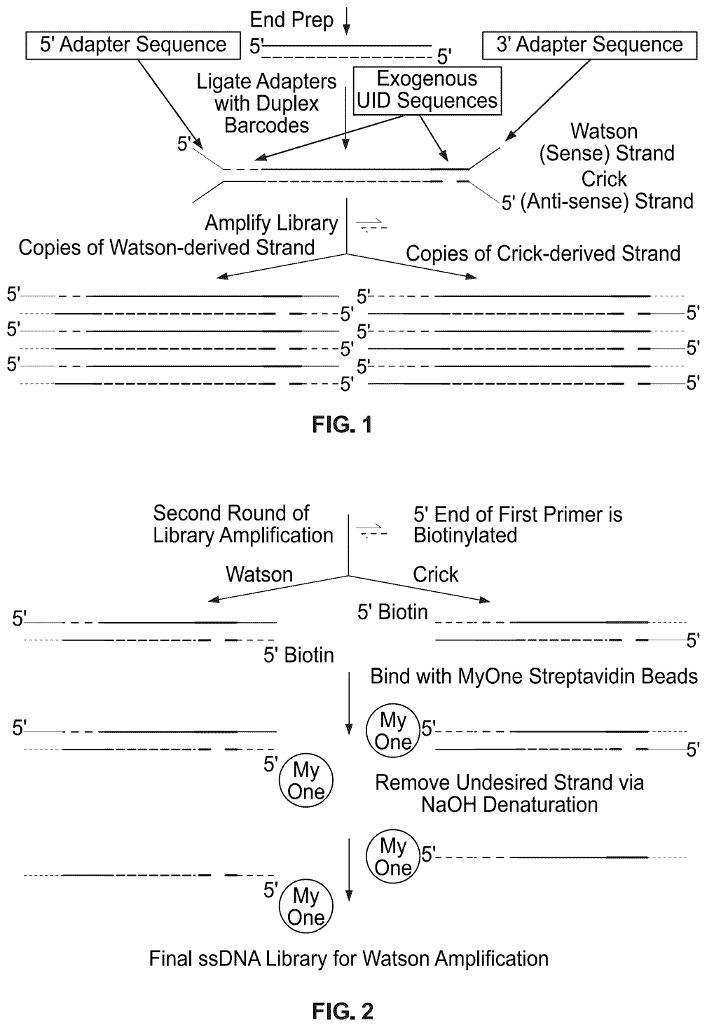

### DETAILED DESCRIPTION

It must be noted that, as used in the specification and the appended claims, the singular forms “a,” “an” and “the” include plural referents unless the context clearly dictates otherwise.

“Nucleotides” and “nt” are used interchangeably herein to generally refer to biological molecules that comprise nucleic acids. Nucleotides can have moieties that contain the known purine and pyrimidine bases. Nucleotides may have other heterocyclic bases that have been modified. Such modifications include, e.g., methylated purines or pyrimidines, acylated purines or pyrimidines, alkylated riboses, or other heterocycles. The terms “polynucleotides,” “nucleic acid,” and “oligonucleotides” can be used interchangeably. They can refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise non-naturally occurring sequences. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

A “primer” is generally a polynucleotide molecule comprising a nucleotide sequence (e.g., an oligonucleotide), generally with a free 3′-OH group, that hybridizes with a template sequence (such as a target polynucleotide, or a primer extension product) and is capable of promoting polymerization of a polynucleotide complementary to the template.

The term “mammal” as used herein includes both humans and non-humans and include but is not limited to humans, non-human primates, canines, felines, murines, bovines, equines, and porcines.

### Overview

This document relates to methods and materials useful for accurately identifying a mutation present in a nucleic acid sample. In some aspects, the method comprises identifying the mutation when it is present on both Watson and Crick strands of a double stranded nucleic acid template. Such methods are particularly useful for distinguishing true mutations from artifacts stemming from, e.g., DNA damage, PCR, and other sequencing artifacts, allowing for the identification of mutations with high confidence.

In some cases, the methods and materials described herein can detect one or more mutations with a low error rate. For example, the methods and materials described herein can be used to detect the presence or absence of a nucleic acid mutation in a nucleic acid template with an error rate of less than about 1% (e.g., less than about 0.1%, less than about 0.05%, or less than about 0.01%). In some cases, the methods and materials described herein can be used to detect the presence or absence of a nucleic acid mutation in a nucleic acid template with an error rate of from about 0.001% to about 0.01%. In some cases, the error rate associated with the identification of one or more mutations in analyte DNA fragments according to a method described herein is no more than 1×10⁻², no more than 1×10⁻³, no more than 1×10⁻⁴, no more than 1×10⁻⁵, no more than 1×10⁻⁶, no more than 5×10⁻⁶, or no more than 1×10⁻⁷. In some cases, the error rate associated with the identification of one or more mutations in analyte DNA fragments according to a method described herein is reduced by at least 2-fold, 4-fold, 5-fold, 10-fold, 20-fold, 30-fold, 40-fold, 50-fold, 60-fold, 70-fold, 80-fold, 90-fold, or 100-fold, as compared to an alternative method of identifying mutations that does not require the mutation to be detected in both Watson and Crick strands of an analyte DNA fragment.

In some embodiments, the alternative method comprises standard molecular barcoding or standard PCR-based molecular barcoding followed by sequencing. In particular embodiments, the alternative method comprises: (a) attaching adapters to a population of double-stranded DNA fragments in an analyte DNA sample, wherein the adapters comprise a unique exogenous UID; (b) performing an initial amplification to amplify the adapter-ligated, double-stranded DNA fragments to produce amplicons; (c) determining sequence reads of one or more amplicons of the one or more of the adapter-ligated, double-stranded DNA fragments; (d) assigning the sequence reads into UID families, wherein each member of a UID family comprises the same exogenous UID sequence; (e) identifying a nucleotide sequence as accurately representing an analyte DNA fragment when a threshold percentage of members of a UID family contain the sequence; and (f) identifying a mutation in the analyte DNA fragment when the sequence identified as accurately representing an analyte DNA fragment differs from a reference sequence that lacks the mutation.

In some cases, the methods and materials described herein can be used to achieve efficient duplex recovery. For example, methods described herein can be used to recover PCR amplification products derived from both the Watson strand and the Crick strand of a double stranded nucleic acid template. In some cases, the methods described herein can be used to achieve at least 50% (e.g., about 50%, about 60%, about 70%, about 75%, about 80%, about 82%, about 85%, about 88%, about 90%, about 93%, about 95%, about 97%, about 99%, or 100%) duplex recovery.

In some cases, the methods and materials described herein can be used to detect a mutation having low allele frequency. For example, methods described herein can be used to detect a mutation having low allele frequency of less than about 1% (e.g., less than about 0.1%, less than about 0.05%, or less than about 0.01%). In some cases, the methods described herein can be used to detect a mutation having low allele frequency of about 0.001%.

In some cases, the methods described herein can be used to detect a mutation that is present in an analyte nucleic acid sample at a frequency of 0.1% or less. In some embodiments, the methods described herein can be used to detect a mutation that is present in an analyte nucleic acid sample at a frequency of 0.1% to 0.00001%. In some embodiments, the methods described herein can be used to detect a mutation that is present in an analyte nucleic acid sample at a frequency of 0.1% to 0.01%.

In some cases, the methods and materials described herein can be used to detect a mutation with minimal (or no) background artifact mutations. In some cases, the methods described herein can be used to detect a mutation with less than 0.01% background artifact mutations. In some cases, the methods described herein can be used to detect a mutation with no background artifact mutations.

In some cases, methods for detecting one or more mutations present on both strands of a double stranded nucleic acid can include generating a duplex sequencing library having a duplex molecular barcode on each end (e.g., the 5′ end and the 3′ end) of each nucleic acid in the library, generating a library of single stranded Watson strand-derived sequences and a library of single stranded Crick-strand derived sequences from the duplex sequencing library, and detecting the presence of one or more mutations present on both strands of the double stranded nucleic acid in each single stranded library. The presence of a first molecular barcode in a 3′ duplex adapter and a second molecular barcode present in a 5′ adapter can be used to distinguish amplification products derived from the Watson strand from amplification products derived from the Crick strand.

In some cases, methods for identifying a mutation comprises: (a) attaching partially double-stranded 3′ adapters to 3′ ends of both Watson and Crick strands of a population of double-stranded DNA fragments in an analyte DNA sample, wherein a first strand of the partially double-stranded 3′ adapter comprises, in the 5′-3′ direction, (i) a first segment, (ii) an exogenous UID sequence, (iii) an annealing site for a 5′ adapter, and (iv) a universal 3′ adapter sequence comprising an R2 sequencing primer site, and wherein the second strand of the partially double-stranded 3′ adapter comprises, in the 5′ to 3′ direction, (i) a segment complementary to the first segment, and (ii) a 3′ blocking group, optionally wherein the second strand is degradable; (b) annealing 5′ adapters to the 3′ adapters via the annealing site, wherein the 5′ adapters comprise, in the 5′ to 3′ direction, (i) a universal 5′ adapter sequence that is not complementary to the universal 3′ adapter sequence and that comprises an R1 sequencing primer site, and (ii) a sequence complementary to the annealing site for the 5′ adapter; (c) performing a nick translation-like reaction to extend the 5′ adapters across the exogenous UID sequence of the 3′ adapters (e.g., using a DNA polymerase) and covalently link the extended 5′ adapter to the 5′ ends of the Watson and Crick strands of the double-stranded DNA fragments (e.g., using a ligase); (d) performing an initial amplification to amplify the adapter-ligated, double-stranded DNA fragments to produce amplicons; (e) determining sequence reads of one or more amplicons of the one or more of the adapter-ligated, double-stranded DNA fragments; (f) assigning the sequence reads into UID families, wherein each member of a UID family comprises the same exogenous UID sequence; (g) assigning sequence reads of each UID family into a Watson subfamily and Crick subfamily based on spatial relationship of the exogenous UID sequence to the R1 and R2 read sequence; (h) identifying a nucleotide sequence as accurately representing a Watson strand of an analyte DNA fragment when a threshold percentage of members of the Watson subfamily contain the sequence; (i) identifying a nucleotide sequence as accurately representing a Crick strand of an analyte DNA fragment when a threshold percentage of members of the Crick subfamily contain the sequence; (j) identifying a mutation in the nucleotide sequence accurately representing the Watson Strand when the sequence accurately representing the Watson Strand differs from a reference sequence that lacks the mutation; (k) identifying a mutation in the nucleotide sequence accurately representing the Crick Strand when the sequence accurately representing the Crick Strand differs from a reference sequence that lacks the mutation; and (l) identifying a mutation in the analyte DNA fragment when the mutation in the nucleotide sequence accurately representing the Watson strand and the mutation in the nucleotide sequence accurately representing the Crick strand are the same mutation.

In some cases, methods for identifying a mutation comprises: (a) attaching adapters to a population of double-stranded DNA fragments, wherein the adapters comprise a double-stranded portion comprising an exogenous UID and a forked portion comprising (i) a single-stranded 3′ adapter sequence comprising an R2 sequencing primer site and (ii) a single-stranded 5′ adapter sequence comprising an R1 sequencing primer site; (b) performing an initial amplification to amplify the adapter-ligated, double-stranded DNA fragments to produce amplicons; (c) selectively amplifying amplicons of Watson strands comprising the target polynucleotide sequence with a first set of Watson target-selective primer pairs, the first set of Watson target-selective primer pairs comprising: (i) a first Watson target-selective primer comprising a sequence complementary to the R2 sequencing primer site of the universal 3′ adapter sequence, and (ii) a second Watson target-selective primer comprising a target-selective sequence, thereby creating target Watson amplification products; (d) selectively amplifying amplicons of Crick strands comprising the same target polynucleotide sequence with a first set of Crick target-selective primer pairs, the first set of Crick target-selective primer pairs comprising: a first Crick target-selective primer comprising a sequence complementary to the R1 sequencing primer site of the universal 5′ adapter sequence, and (ii) a second Crick target-selective primer comprising the same target-selective sequence as the second Crick target-selective primer sequence, thereby creating target Crick amplification products; (e) determining sequence reads of the target Watson amplification products and the target Crick amplification products; (f) assigning the sequence reads into UID families, wherein each member of a UID family comprises the same exogenous UID sequence; (g) assigning sequence reads of each UID family into a Watson subfamily and Crick subfamily based on spatial relationship of the exogenous UID sequence to the R1 and R2 read sequence; (h) identifying a nucleotide sequence as accurately representing a Watson strand of an analyte DNA fragment when a threshold percentage of members of the Watson family contain the sequence; (i) identifying a nucleotide sequence as accurately representing a Crick strand of an analyte DNA fragment when a threshold percentage of members of the Crick family contain the sequence; and (j) identifying a mutation in the analyte DNA fragment when the nucleotide sequence accurately representing the Watson strand and the nucleotide sequence accurately representing the Crick strand both contain the same mutation.

In some cases, the methods and materials described herein can be used to independently assess each strand of a double stranded nucleic acid. For example, when a nucleic acid mutation is identified in independently assessed strands of a double stranded nucleic acid as described herein, the materials and methods described herein can used to determine from which strand of the double stranded nucleic acid the nucleic acid mutation originated.

Any appropriate method can be used to generate a duplex sequencing library. As used herein a duplex sequencing library is a plurality of nucleic acid fragments including a duplex molecular barcode on at one end (e.g., the 5′ end and/or the 3′ end) of each nucleic acid fragment in the library and can allow both strands of a double stranded nucleic acid to be sequenced. In some cases, a nucleic acid sample can be fragmented to generate nucleic acid fragments, and the generated nucleic acid fragments can be used to generate a duplex sequencing library. Nucleic acid fragments used to generate a duplex sequencing library can also be referred to herein as input nucleic acid. For example, when nucleic acid fragments used to generate a duplex sequencing library are DNA fragments, the DNA fragments can also be referred to herein as input DNA. A duplex sequencing library can include any appropriate number of nucleic acid fragments. In some cases, generating a duplex sequencing library can include fragmenting a nucleic acid template and ligating adapters to each end of each nucleic acid fragment in the library.

### Analyte Nucleic Acid Sample

Nucleic acid templates in an analyte nucleic acid sample can comprise any type of nucleic acid (e.g., DNA, RNA, and DNA/RNA hybrids). In some cases, a nucleic acid template can be a double-stranded DNA template. Examples of nucleic acid can be used as a template for the methods described herein include, without limitation, genomic DNA, circulating free DNA (cfDNA; e.g., circulating tumor DNA (ctDNA), and cell-free fetal DNA (cffDNA)).

In some embodiments, the nucleic acid templates in the nucleic acid sample are nucleic acid fragments, e.g., DNA fragments. In some embodiments, the ends of a DNA fragment represent unique sequences which can be used as an endogenous unique identifier of the fragment. In some embodiments, the fragments are manually produced. In some embodiments, the fragments are produced by shearing, e.g., enzymatic shearing, shearing by chemical means, acoustic shearing, nebulization, centrifugal shearing, point-sink shearing, needle shearing, sonication, restriction endonucleases, non-specific nucleases (e.g., DNase I), and the like. In some embodiments, the fragments are not manually produced. In some embodiments, the fragments are from a cfDNA sample.

In some embodiments, a nucleic acid fragment in the nucleic acid sample has a length. The length may be about 4-1000 nucleotides. The length may be about 60-300 nucleotides. The length may be about 60-200 nucleotides. Then length can be about 140-170 nucleotides. The length may be less than 500, less than 400, less than 300, less than 250 nt, or less than 200 nt.

In some embodiments, ends of nucleic acid templates are used as endogenous UIDs. A skilled artisan may determine the length of the endogenous UID needed to uniquely identify a nucleic acid template, using factors such as, e.g., overall template length, complexity of nucleic acid templates in a partition or starting nucleic acid sample, and the like. In some embodiments, 10-500 nucleotides of the ends of nucleic acid templates are used as endogenous UIDs. In some embodiments, 15-100 nucleotides of the ends of nucleic acid templates are used as endogenous UIDs. In some embodiments, 15-40 nucleotides of the ends of nucleic acid templates are used as endogenous UIDs. In some embodiments, at least 10 nucleotides of the ends of nucleic acid templates are used as endogenous UIDs. In some embodiments, at least 15 nucleotides of the ends of nucleic acid templates are used as endogenous UIDs. In some embodiments, only one end of a nucleic acid template is used as an endogenous UID.

In some embodiments, nucleic acid templates comprise one or more target polynucleotides. The terms “target polynucleotide,” “target region,” “nucleic acid template of interest,” “desired locus,” “desired template,” or “target,” are used interchangeably herein to refer to a polynucleotide of interest under study. In certain embodiments, a target polynucleotide contains one or more sequences that are of interest and under study. A target polynucleotide can comprise, for example, a genomic sequence. The target polynucleotide can comprise a target sequence whose presence, amount, and/or nucleotide sequence, or changes in these, are desired to be determined.

The target polynucleotide can be a region of gene associated with a disease. In some embodiments, the gene is a druggable target. The term “druggable target”, as used herein, generally refers to a gene or cellular pathway that is modulated by a disease therapy. The disease can be cancer. Accordingly, the gene can be a known cancer-related gene.

In some embodiments, the input nucleic acid, also referred to herein as the nucleic acid sample, was obtained from a biological sample. The biological sample may be obtained from a subject. In some embodiments, the subject is a mammal. Examples of mammals from which nucleic acid can be obtained and used as a nucleic acid template in the methods described herein include, without limitation, humans, non-human primates (e.g., monkeys), dogs, cats, sheep, rabbits, mice, hamsters, and rats. In some embodiments, the subject is a human subject. In some embodiments, the subject is a plant.

Biological samples include but are not limited to plasma, serum, blood, tissue, tumor sample, stool, sputum, saliva, urine, sweat, tears, ascites, bronchoaveolar lavage, semen, archeologic specimens and forensic samples. In particular embodiments, the biological sample is a solid biological sample, e.g., a tumor sample. In some embodiments, the solid biological sample is processed. The solid biological sample may be processed by fixation in a formalin solution, followed by embedding in paraffin (e.g., is a FFPE sample). Processing can alternatively comprise freezing of the sample prior to conducting the probe-based assay. In some embodiments, the sample is neither fixed nor frozen. The unfixed, unfrozen sample can be, by way of example only, stored in a storage solution configured for the preservation of nucleic acid.

In some embodiments, the biological sample is a liquid biological sample. Liquid biological samples include, but are not limited to plasma, serum, blood, sputum, saliva, urine, sweat, tears, ascites, bronchoaveolar lavage, and semen. In some embodiments, the liquid biological sample is cell free or substantially cell free. In particular embodiments, the biological sample is a plasma or serum sample. In some embodiments, the liquid biological sample is a whole blood sample. In some embodiments, the liquid biological sample comprises peripheral mononuclear blood cells.

In some embodiments, a nucleic acid sample has been isolated and purified from the biological sample. Nucleic acid can be isolated and purified from the biological sample using any means known in the art. For example, a biological sample may be processed to release nucleic acid from cells, or to separate nucleic acids from unwanted components of the biological sample (e.g., proteins, cell walls, other contaminants). For example, nucleic acid can be extracted from the biological sample using liquid extraction (e.g., Trizol, DNAzol) techniques. Nucleic acid can also be extracted using commercially available kits (e.g., Qiagen DNeasy kit, QIAamp kit, Qiagen Midi kit, QIAprep spin kit).

In some embodiments, the biological sample comprises low amounts of nucleic acid. In some embodiments, the biological sample comprises less than about 500 nanograms (ng) of nucleic acid. For example, the biological sample comprises from about 30 ng to about 40 ng of nucleic acid.

Nucleic acid can be concentrated by known methods, including, by way of example only, centrifugation. Nucleic acid can be bound to a selective membrane (e.g., silica) for the purposes of purification. Nucleic acid can also be enriched for fragments of a desired length, e.g., fragments which are less than 1000, 500, 400, 300, 200 or 100 base pairs in length. Such an enrichment based on size can be performed using, e.g., PEG-induced precipitation, an electrophoretic gel or chromatography material (Huber et al. (1993) Nucleic Acids Res. 21:1061-6), gel filtration chromatography, TSK gel (Kato et al. (1984) J. Biochem, 95:83-86), which publications are hereby incorporated by reference.

Polynucleotides extracted from a biological sample can be selectively precipitated or concentrated using any methods known in the art.

In some embodiments, the nucleic acid sample comprises less than about 35 ng of nucleic acid. For example, the nucleic acid sample comprises can include from about 1 ng to about 35 ng of nucleic acid (e.g., from about 1 ng to about 30 ng, from about 1 ng to about 25 ng, from about 1 ng to about 20 ng, from about 1 ng to about 15 ng, from about 1 ng to about 10 ng, from about 1 ng to about 5 ng, from about 5 ng to about 35 ng, from about 10 ng to about 35 ng, from about 15 ng to about 35 ng, from about 20 ng to about 35 ng, from about 25 ng to about 35 ng, from about 30 ng to about 35 ng, from about 5 ng to about 30 ng, from about 10 ng to about 25 ng, from about 15 ng to about 20 ng, from about 5 ng to about 10 ng, from about 10 ng to about 15 ng, from about 15 ng to about 20 ng, from about 20 ng to about 25 ng, or from about 25 ng to about 30 ng of nucleic acid). In some cases, a nucleic acid sample can include nucleic acid from a genome that includes more than about several hundred nucleotides of nucleic acid.

In some cases, a nucleic acid sample can be essentially free of contamination. For example, when a nucleic acid sample is a cfDNA template, the cfDNA can be essentially free of genomic DNA contamination. In some cases, a cfDNA sample that is essentially free of genomic DNA contamination can include minimal (or no) high molecular weight (e.g., >1000 bp) DNA. In some cases, methods described herein can include determining whether a nucleic acid sample is essentially free of contamination. Any appropriate method can be used to determine whether a nucleic acid sample is essentially free of contamination. Examples of methods that can be used to determine whether a nucleic acid sample is essentially free of contamination include, for example, a TapeStation system, and a Bioanalyzer. For example, when using a TapeStation system and/or a Bioanalyzer to determine whether a cfDNA sample is essentially free of genomic DNA contamination, a prominent peak at ˜180 bp (e.g., corresponding to mononucleosomal DNA) can be used to indicate that the nucleic acid sample is essentially free of genomic DNA contamination.

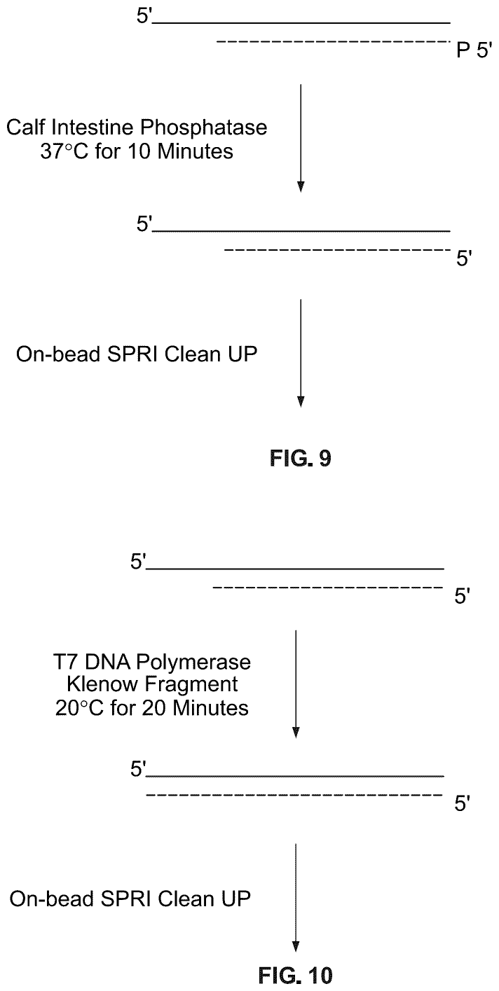

In some cases, nucleic acid fragments that can be used to generate a duplex sequencing library (e.g., prior to attaching a 3′ duplex adapter to the 3′ ends of the nucleic acid fragments) can be end-repaired. Any appropriate method can be used to end-repair a nucleic acid template. For example, blunting reactions (e.g., blunt end ligations) and/or dephosphorylation reactions can be used to end-repair a nucleic acid template. In some cases, blunting can include filling in a single stranded region. In some cases, blunting can include degrading a single stranded region. In some cases, blunting and dephosphorylation reactions can be used to end-repair a nucleic acid template as shown in FIG. 9 and/or FIG. 10.

### Adapters

In some embodiments, the method comprises attaching adapters to a population of double-stranded DNA fragments to produce a population of adapter-attached, double-stranded DNA fragments.

In some embodiments, the adapters comprise a double-stranded portion comprising an exogenous UID and a forked portion comprising (i) a single-stranded 3′ adapter sequence and (ii) a single-stranded 5′ adapter sequence. In some embodiments, the single-stranded 3′ adapter sequence is not complementary to the single-stranded 5′ adapter sequence. In some embodiments, the 3′ adapter sequence comprises a second (e.g., R2) sequencing primer site and the 5′ adapter sequence comprises a first (e.g., R1) sequencing primer site. It is to be understood that an “R1” and “R2” sequencing primer sites are used by sequencing systems that produce paired end reads, e.g., reads from opposite ends of a DNA fragment to be sequenced. In some embodiments, the R1 sequencing primer is used to produce a first population of reads from first ends of DNA fragments, and the R2 sequencing primer is used to produce a second population of reads from the opposite ends of the DNA fragments. The first population is referred to herein as “R1” or “Read 1” reads. The second population is referred to herein as “R2” or “Read 2” reads. The R1 and R2 reads can be aligned as “read pairs” or “mate pairs” corresponding to each strand of a double-stranded analyte DNA fragment.

Certain sequencing systems, e.g., Illumina, utilizes what they refer to as “R1” and “R2” primers, and “R1” and “R2” reads. It should be noted that the terms “R1” and “R2”, and “Read 1” and “Read 2”, for the purposes of this application, are not limited to how they are referenced in relation to a particular sequencing platform. For example, if an Illumina sequencer is used, the “R2” primer and corresponding R2 read disclosed herein may refer to the Illumina “R2” primer and read, or may refer to the Illumina “R1” primer and read, so long as the “R1” primer and corresponding R1 read disclosed herein refers to the other Illumina primer and read. To clarify, in some embodiments wherein an “R2” primer provided herein is the Illumina “R1” primer producing “R1” reads, the corresponding “R1” primer provided herein is the Illumina “R2” primer producing “R2” reads. To clarify, in some embodiments wherein an “R2” primer provided herein is the Illumina “R2” primer providing “R2” reads, the “R1” primer provided herein is the Illumina “R1” primer providing R1 reads.

In some embodiments, the exogenous UID is unique to each double-stranded DNA fragment in the nucleic acid sample. In some embodiments, the exogenous UID is not unique to each double-stranded DNA fragment.

In some embodiments, the exogenous UID has a length. The length can be about 2-4000 nt. The length can be about 6-100 nt. The length can be about 8-50 nt. The length can be about 10-20 nt. The length can be about 12-14 nt. In some embodiments, the length of the exogenous UID is sufficient to uniquely barcode the molecules and the length/sequence of the exogenous UID does not interfere with the downstream amplification steps.

In some embodiments, the exogenous UID sequence does not exist in the nucleic acid template. In some embodiments, the exogenous UID sequence does not exist in a desired template harboring a desired locus. Such unique sequences can be randomly generated, e.g., by a computer readable medium, and selected by BLASTing against known nucleotide databases such as, e.g., EMBL, GenBank, or DDBJ. In some embodiments, an exogenous UID sequence exists in a nucleic acid template. In such cases, the position of the exogenous UID sequence in the sequence read is used to distinguish the exogenous UID sequence from a sequence within the nucleic acid template.

In some embodiments, the exogenous UID sequence is random. In some embodiments, the exogenous UID sequence is a random N-mer. For example, if the exogenous UID sequence has a length of six nt, then it may be a random hexamer. If the exogenous UID sequence has a length of 12 nt, then it may be a random 12-mer.

Exogenous UIDs may be made using random addition of nucleotides to form a sequence having a length to be used as an identifier. At each position of addition, a selection from one of four deoxyribonucleotides may be used. Alternatively a selection from one of three, two, or one deoxyribonucleotides may be used. Thus the UID may be fully random, somewhat random, or non-random in certain positions.

In some embodiments, the exogenous UIDs are not random N-mers, but are selected from a predetermined set of exogenous UID sequences.

Exemplary exogenous UIDs suitable for use in the methods disclosed herein are described in PCT/US2012/033207, which is hereby incorporated by reference in its entirety.

Forked adapters described herein may be attached to double-stranded DNA fragments by any means known in the art.

In some embodiments, the forked adapters are attached to double-stranded DNA fragments by: (a) attaching partially double-stranded 3′ adapters to 3′ ends of both Watson and Crick strands of a population of double-stranded DNA fragments, wherein a first strand of the partially double-stranded 3′ adapter comprises, in the 5′-3′ direction, (i) a first segment, (ii) an exogenous UID sequence, (iii) an annealing site for a 5′ adapter, and (iv) a universal 3′ adapter sequence comprising an R2 sequencing primer site, and wherein the second strand of the partially double-stranded 3′ adapter comprises, in the 5′ to 3′ direction, (i) a segment complementary to the first segment, and (ii) a 3′ blocking group, optionally wherein the second strand is degradable; (b) annealing 5′ adapters to the 3′ adapters via the annealing site, wherein the 5′ adapters comprise, in the 5′ to 3′ direction, (i) a universal 5′ adapter sequence that is not complementary to the universal 3′ adapter sequence and that comprises an R1 sequencing primer site, and (ii) a sequence complementary to the annealing site for the 5′ adapter; and (c) performing a nick translation-like reaction) to extend the 5′ adapters across the exogenous UID sequence of the 3′ adapters (e.g., using a DNA polymerase) and covalently link the extended 5′ adapter to the 5′ ends of the Watson and Crick strands of the double-stranded DNA fragments (e.g., using a ligase).

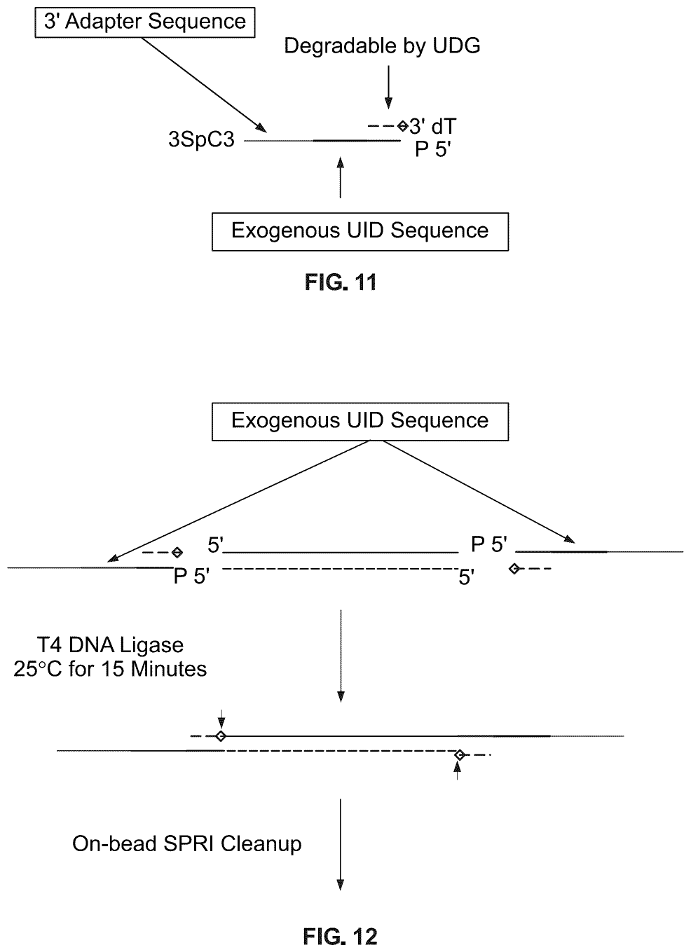

In some embodiments, the forked adapters are attached to double-stranded DNA fragments by: (a) attaching a 3′ duplex adapter to 3′ ends of both Watson and Crick strands of a population of double-stranded DNA fragments. A 3′ duplex adapter, also referred to herein as a partially double stranded 3′ adapter, as described herein is an oligonucleotide complex including a molecular barcode that can have a first oligonucleotide (also referred to herein as “first strand”) annealed (hybridized) to a second oligonucleotide (also referred to herein as “second strand”) such that a portion (e.g., first portion) of the 3′ duplex adapter is double stranded and a portion (e.g., a second portion) of the 3′ duplex adapter is single stranded. In some cases, a first oligonucleotide of a 3′ duplex adapter described herein comprises a first segment comprising nucleotides that are complementary to nucleotides present in a second oligonucleotide of the 3′ duplex adapter (e.g., such that the first oligonucleotide of the 3′ duplex adapter and the second oligonucleotide of the 3′ duplex adapter can anneal at the complementary region). An exemplary structure of a 3′ duplex adapter can be as shown in FIG. 11.

The first oligonucleotide of a 3′ duplex adapter described herein can be an oligonucleotide that includes a 5′ phosphate and a molecular barcode. The first oligonucleotide of a 3′ duplex adapter described herein can include any appropriate number of nucleotides. Any appropriate molecular barcode can be included in a first oligonucleotide of a 3′ duplex adapter described herein. In some cases, a molecular barcode can include a random sequence. In some cases, a molecular barcode can include a fixed sequence. Examples of molecular barcodes that can be included in a first oligonucleotide of a 3′ duplex adapter described herein include, without limitation, IDT 8, IDT 10, ILMN 8, ILMN 10 as available from Integrated DNA technologies. Any appropriate type of molecular barcode can be used. In some cases, a molecular barcode comprise an exogenous UID sequence. Exogenous UIDs are described herein. Examples of oligonucleotides that include a 5′ phosphate and a molecular barcode and can be included in a first oligonucleotide of a 3′ duplex adapter described herein include, without limitation, ATAAAACGACGGCNNNNNNNNNNNNNNAGATCGGAAGAGCACACGTCTGAACT CCAG*T*C (with the asterisks representing phosphorothioate bonds; SEQ ID NO:1), where NNNNNNNNNNNNNN (SEQ ID NO:2) is a molecular barcode, and where the number of nucleotides in the molecular barcode can be from 0 to about 25.

In some embodiments, the first oligonucleotide of the 3′ duplex adapter comprises an annealing site for a 5′ adapter.

In some embodiments, the first oligonucleotide of the 3′ duplex adapter comprises a universal 3′ adapter sequence. In some embodiments, the universal 3′ adapter sequence comprises an R2 sequencing primer site.

In some cases, a first oligonucleotide of a 3′ duplex adapter described herein also can include one or more features to prevent or reduce extension during a PCR. A feature that can prevent or reduce extension during a PCR can be any type of feature (e.g., a chemical modification). Examples of feature that can prevent or reduce extension during a PCR and can be included in a first oligonucleotide of a 3′ duplex adapter described herein include, without limitation, 3SpC3 and 3Phos. A feature that can prevent or reduce extension during a PCR can be incorporated into a first oligonucleotide of a 3′ duplex adapter described herein in any appropriate position within the oligonucleotide. In some case, a molecule that can prevent or reduce extension during a PCR can be incorporated internally within the oligonucleotide. In some case, a molecule to prevent or reduce extension during a PCR can be incorporated at and end (e.g., the 5′ end) of the oligonucleotide.

In particular embodiments, the first oligonucleotide of the 3′ duplex adapter comprises a 5′ phosphate, a first segment comprising nucleotides that are complementary to nucleotides present in a second oligonucleotide of the 3′ duplex adapter, an exogenous UID sequence, an annealing site for a 5′ adapter, and a universal 3′ adapter sequence.

The second oligonucleotide of a 3′ duplex adapter described herein can be an oligonucleotide that includes a blocked 3′ group (e.g., to reduce or eliminate dimerization of two adapters). The second oligonucleotide of a 3′ duplex adapter described herein can include any appropriate number of nucleotides. In some embodiments, the second oligonucleotide of the 3′ duplex adapter is complementary to the first segment of the first oligonucleotide of the 3′ duplex adapter. An exemplary oligonucleotide that includes a blocked 3′ group and can be included in a second oligonucleotide of a 3′ duplex adapter described herein includes, without limitation, GCCGUCGUUUUAdT (SEQ ID NO:3).

The second oligonucleotide of a 3′ duplex adapter described herein can be degradable. Any appropriate method can be used to degrade a second oligonucleotide of a 3′ duplex adapter described herein. For example, UDG can be used to degrade a second oligonucleotide of a 3′ duplex adapter described herein.

In some cases, a 3′ duplex adapter described herein can include a first oligonucleotide including the sequence ATAAAACGACGGCNNNNNNNNNNNNNNAGATCGGAAGAGCACACGTCTGAACT CCAG*T*C/3SpC3 (SEQ ID NO:1) annealed to a second oligonucleotide including the sequence GCCGUCGUUUUAdT (SEQ ID NO:3).

In some cases, a 3′ duplex adapter described herein can include a commercially available adapter. An exemplary commercially available adapters that can be used as (or can be used to generate) a 3′ duplex adapter described herein includes, without limitation, adapters in an Accel-NGS 2S DNA Library Kit (Swift Biosciences, cat. #21024). In some cases, a 3′ duplex adapter described herein can be as described in Example 1.

The 3′ adapters can be attached (e.g., covalently attached) to 3′ ends of the double-stranded DNA fragments using any appropriate method. In some embodiments, the 3′ adapters are attached by ligation. In some embodiments, the ligation comprises use of a ligase. Examples of ligases that can be used to attach a 3′ adapter to the 3′ ends of each nucleic acid fragment include, without limitation, T4 DNA ligases, E. coli ligases (e.g., Enzyme Y3), CircLigase I, CircLigase II, Taq-Ligase, T3 Ligase, T7 Ligase, and 9N Ligase.

Once the 3′ duplex adapter is attached (e.g., covalently attached) to the 3′ ends of each nucleic acid fragment, the second oligonucleotide of a 3′ duplex adapter described herein can be degraded, and a 5′ adapter can be attached (e.g., covalently attached) to the 5′ ends of each nucleic acid fragment. In some embodiments, the 5′ adapter sequence is not complementary to the first oligonucleotide of the 3′ adapter. In some embodiments, the 5′ adapter sequence comprises, in the 5′ to 3′ direction, an R1 sequencing primer site and a sequence complementary to the annealing site of the 3′ adapter.

In some embodiments, the attaching of the 5′ adapter comprises annealing the 5′ adapter to the 3′ adapter via the annealing site.

A 5′ adapter can anneal to a nucleic acid fragment upstream of a molecular barcode on a 3′ duplex adapter such that a gap (e.g., single stranded nucleic acid fragment) containing a portion (e.g., a molecular barcode) of the 3′ duplex adapter is present on the nucleic acid fragment. The gap containing a portion of the 3′ duplex adapter can be filled in (e.g., to generate a double stranded nucleic acid fragment). Any appropriate method can be used to fill in the single stranded gap. Examples of methods that can be used to fill in a single stranded gap on a nucleic acid fragment include, without limitation, polymerases such as DNA polymerases (e.g., Taq polymerases such as a Taq-B polymerase) and nick-translation reactions (e.g., including both a ligase such as an E. coli ligase and a polymerase such as a DNA polymerase). In cases where filling in a single stranded gap on a nucleic acid fragment includes providing a polymerase, the method also can include providing deoxyribonucleotide triphosphates (dNTPs; e.g., dATP, dGTP, dCTP, and dTTP). In some cases, attaching a 5′ adapter to the 5′ ends of each nucleic acid fragment and filling in the single stranded gap can be done concurrently (e.g., in a single reaction tube).

In some cases, alternative methods can be used to attach the adapters to templates. For example, nucleic acid fragments can be treated with single strand nucleases (e.g., to digest overhangs) followed by ligation can be used to prepare a duplex sequencing library. For example, a single nucleotide can be added to the 3′ ends of each nucleic acid fragment and adapters (e.g., containing a molecular barcode) containing a complementary base at the 5′ end can be ligated to each nucleic acid fragment to prepare a duplex sequencing library of adapter-attached templates.

### Initial Amplification of the Adapter-Attached Templates

Following adapter attachment, the adapter-attached templates can be amplified (e.g., PCR amplified) in an initial amplification reaction. Any appropriate method can be used to amplify the adapter-attached templates. An exemplary method that can be used to amplify the adapter-attached templates includes, without limitation, whole-genome PCR.

Any appropriate primer pair can be used to amplify the adapter-attached templates. In some cases, a universal primer pair can be used. A primer can include, without limitation from about 12 nucleotides to about 30 nucleotides. Examples of primer pairs that can be used to amplify the adapter-attached templates as described herein include, without limitation, those described in Example 1 and/or Example 2.

Any appropriate PCR conditions can be used in the initial amplification. PCR amplification can include a denaturing phase, an annealing phase, and an extension phase. Each phase of an amplification cycle can include any appropriate conditions. In some cases, a denaturing phase can include a temperature of about 90° C. to about 105° C. (e.g., about 94° C. to about 98° C.), and a time of about 1 second to about 5 minutes (e.g., about 10 seconds to about 1 minute). For example, a denaturing phase can include a temperature of about 98° C. for about 10 seconds. In some cases, an annealing phase can include a temperature of about 50° C. to about 72° C., and a time of about 30 seconds to about 90 seconds. In some cases, an extension phase can include a temperature of about 55° C. to about 80° C., and a time of about 15 seconds per kb of the amplicon to be generated to about 30 seconds per kb of the amplicon to be generated. In some cases, annealing and extension phases can be performed in a single cycle. For example, an annealing and phase extension phase can include a temperature of about 65° C. for about 75 seconds.

PCR conditions used in the initial amplification can include any appropriate number of PCR amplification cycles. In some cases, PCR amplification can include from about 1 to about 50 cycles. In some embodiments, the PCR amplification comprises no more than 11 cycles. In some embodiments, the PCR amplification comprises no more than 7 cycles. In some embodiments, the PCR amplification comprises no more than 5 cycles.

In some cases, when PCR conditions include a heat-activated polymerase, PCR amplification also can include an initialization step. For example, PCR amplification can include an initialization step prior to performing the PCR amplification cycles. In some cases, an initialization step can include a temperature of about 94° C. to about 98° C., and a time of about 15 seconds to about 1 minute. For example, an initialization step can include a temperature of about 98° C. for about 30 seconds.

In some cases, PCR amplification also can include a hold step. For example, PCR amplification can include a hold step after performing the PCR amplification cycles, an optionally after performing any final extension step. In some case, a hold step can include a temperature of about 4° C. to about 15° C., for an indefinite amount of time.

In some cases, a duplex sequencing library generated as described herein (e.g., an amplified duplex sequencing library) can be purified. Any appropriate method can be used to purify a duplex sequencing library. An exemplary method that can be used to purify a duplex sequencing library includes, without limitation, magnetic beads (e.g., solid phase reversible immobilization (SPRI) magnetic beads).

Optional ssDNA Library Prep

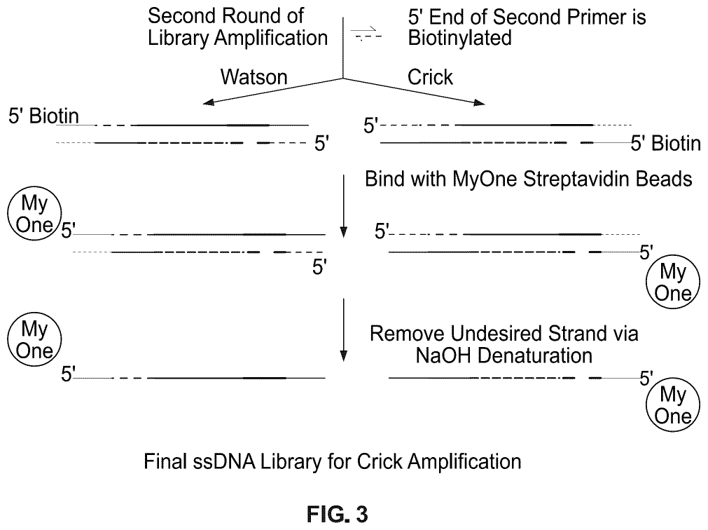

In some cases, a duplex sequencing library can be used to generate a library of single stranded Watson strand-derived sequences and a library of single stranded Crick-strand derived sequences. Generating a library of single stranded Watson strand-derived sequences and a library of single stranded Crick-strand derived sequences can minimize non-specific amplification (e.g., from a primer complementary to a ligated sequence such as a 3′ duplex adapter or a 5′ adapter). Any appropriate method can be used to generate a library of single stranded Watson strand-derived sequences and a library of single stranded Crick-strand derived sequences (e.g., from a duplex sequencing library generated as described herein). In some cases, a library of single stranded Watson strand-derived sequences and a library of single stranded Crick-strand derived sequences can be generated from an amplified duplex sequencing library by dividing the amplification products into at least two aliquots, and subjecting each aliquot to a PCR amplification where the Watson strand is amplified from a first aliquot, and the Crick strand is amplified from a second aliquot. For example, a first aliquot of amplification products from an amplified duplex sequencing library can be subjected to a PCR amplification using a primer pair where a first primer is biotinylated and a second primer is non-biotinylated to generate a single stranded library of Watson strands, and a second aliquot of amplification products from an amplified duplex sequencing library can be subjected to a PCR amplification using a primer pair where a first primer is non-biotinylated and a second primer is biotinylated to generate a single stranded library of Crick strands. In some cases, a library of single stranded Watson strand-derived sequences and a library of single stranded Crick-strand derived sequences can be generated as shown in FIG. 2 and FIG. 3.

Any appropriate method can be used to generate a library of single stranded Watson strand-derived sequences and a library of single stranded Crick-strand derived sequences from an amplified duplex sequencing library. For example, amplification products from an amplified duplex sequencing library can be separated into a first PCR amplification and a second PCR amplification in which only one of the two primers in the PCR primer pair is tagged. For example, a first PCR amplification can use a primer pair that includes a primer (e.g., a first primer) that is tagged and a primer (e.g., a second primer) that is not tagged, and a second PCR amplification can use a primer pair that includes a primer (e.g., a first primer) that is not tagged and a primer (e.g., a second primer) that is tagged. A primer tag can be any tag that enables a PCR amplification product generated from the tagged primer to be recovered. In some cases, a tagged primer can be a biotinylated primer, and a PCR amplification produce generated from the biotinylated primer can be recovered using streptavidin. For example, a library of single stranded Watson strand-derived sequences and a library of single stranded Crick-strand derived sequences can be generated in a PCR amplification using a primer pair including a biotinylated primer and a non-biotinylated primer. In some cases, a tagged primer can be a phosphorylated primer, and a PCR amplification produce generated from the phosphorylated primer can be recovered using a lambda nuclease. For example, a library of single stranded Watson strand-derived sequences and a library of single stranded Crick-strand derived sequences can be generated in a PCR amplification using a primer pair including a phosphorylated primer and a non-phosphorylated primer.

Any appropriate primer pair can be used to generate a library of single stranded Watson strand-derived sequences and a library of single stranded Crick-strand derived sequences (e.g., from a duplex sequencing library generated as described herein). A primer can include, without limitation, from about 12 nucleotides to about 30 nucleotides. In some cases, a primer pair can include at least one primer that can target (e.g., target and bind to) an adapter sequence (e.g., an adapter sequence containing a molecular barcode) present in an amplification product generated as described herein (e.g., by ligating a 3′ duplex adapter including a first molecular barcode and a 5′ adapter including a second molecular barcode to a nucleic acid fragment in a duplex sequencing library prior to the amplification). Examples of primer pairs that can be used to generate a library of single stranded Watson strand-derived sequences and a library of single stranded Crick-strand derived sequences as described herein include, without limitation, a P5 primer and a P7 primer.

Any appropriate PCR conditions can be used to generate a library of single stranded Watson strand-derived sequences and a library of single stranded Crick-strand derived sequences (e.g., from a duplex sequencing library generated as described herein). PCR amplification can include a denaturing phase, an annealing phase, and an extension phase. Each phase of an amplification cycle can include any appropriate conditions. In some cases, a denaturing phase can include a temperature of about 90° C. to about 105° C., and a time of about 1 second to about 5 minutes. For example, a denaturing phase can include a temperature of about 98° C. for about 10 seconds. In some cases, an annealing phase can include a temperature of about 50° C. to about 72° C., and a time of about 30 seconds to about 90 seconds. In some cases, an extension phase can include a temperature of about 55° C. to about 80° C., and a time of about 15 seconds per kb of the amplicon to be generated to about 30 seconds per kb of the amplicon to be generated In some cases, an extension phase reflects the processivity of the polymerase that is used. In some cases, annealing and extension phases can be performed in a single cycle. For example, an annealing and phase extension phase can include a temperature of about 65° C. for about 75 seconds.

PCR conditions used to generate a library of single stranded Watson strand-derived sequences and a library of single stranded Crick-strand derived sequences (e.g., from a duplex sequencing library generated as described herein) can include any appropriate number of PCR amplification cycles. In some cases, PCR amplification can include, without limitation, from about 1 to about 50 cycles. For example, PCR amplification can include about 4 amplification cycles.

In some cases, when PCR conditions include a heat-activated polymerase, PCR amplification also can include an initialization step. For example, PCR amplification can include an initialization step prior to performing the PCR amplification cycles. In some cases, an initialization step can include a temperature of about 94° C. to about 98° C., and a time of about 15 seconds to about 1 minute. For example, an initialization step can include a temperature of about 98° C. for about 30 seconds.

In some cases, PCR amplification also can include a hold step. For example, PCR amplification can include a hold step after performing the PCR amplification cycles, an optionally after performing any final extension step. In some case, a hold step can include a temperature of about 4° C. to about 15° C., for an indefinite amount of time.

Any appropriate method can be used to separate double stranded amplification products into single stranded amplification products. In some cases, a double stranded amplification products can be denatured to separate double stranded amplification products into two single stranded amplification products. Examples of methods that can be used to separate a double stranded amplification product into single stranded amplification products include, without limitation, heat denaturation, chemical (e.g., NaOH) denaturation, and salt denaturation.

Following PCR amplification, the tagged PCR amplification products can be recovered. Any appropriate method can be used to recover tagged PCR amplification products generated using a tagged primer. In cases where a tagged primer is a biotinylated primer, the biotinylated amplification products (e.g., generated from the biotinylated primer) can be recovered using streptavidin (e.g., streptavidin-functionalized beads). For example, when an amplified duplex sequencing library is further amplified in a first PCR amplification using a primer pair that includes a first biotinylated primer and a second non-biotinylated primer, and a second PCR amplification using a primer pair that includes a first non-biotinylated primer and a second biotinylated primer, the biotinylated amplification products generated from the first PCR amplification can be bound to streptavidin-functionalized beads (e.g., a first set of streptavidin-functionalized beads) and the biotinylated amplification products generated from the second PCR amplification can be bound to streptavidin-functionalized beads (e.g., a first second of streptavidin-functionalized beads), and the double stranded amplification products can be separated (e.g., denatured) into single strands of the amplification products. In some cases, recovering biotinylated PCR amplification products also can include releasing the biotinylated PCR amplification products from the streptavidin (e.g., the streptavidin-functionalized beads). Separating the double stranded amplification products generated by a first PCR amplification using a primer pair that includes a first biotinylated primer and a second non-biotinylated primer, and a second PCR amplification using a primer pair that includes a first non-biotinylated primer and a second biotinylated primer, can allow single stranded amplification products generated from the biotinylated primers to remain bound to the streptavidin-functionalized beads while single stranded amplification products generated from the non-biotinylated primers can be denatured (e.g., denatured and degraded) from the streptavidin-functionalized beads, thereby generating a library of single stranded Watson strand-derived sequences and a library of single stranded Crick-strand derived sequences of the duplex sequencing library.

In cases where a tagged primer is a phosphorylated primer, the phosphorylated amplification products (e.g., generated from the phosphorylated primer) can be recovered using an exonuclease (e.g., a lambda exonuclease). For example, when an amplified duplex sequencing library is further amplified in a first PCR amplification using a primer pair that includes a first phosphorylated primer and a second non-phosphorylated primer, and a second PCR amplification using a primer pair that includes a first non-phosphorylated primer and a second phosphorylated primer, the double stranded amplification products can be separated into single strands of the amplification products. Separating the double stranded amplification products generated by a first PCR amplification using a primer pair that includes a first phosphorylated primer and a second non-phosphorylated primer, and a second PCR amplification using a primer pair that includes a first non-phosphorylated primer and a second phosphorylated primer, can allow single stranded amplification products generated from the non-phosphorylated primers to be recovered while single stranded amplification products generated from the phosphorylated primers can be degraded by a lambda exonuclease, thereby generating a library of single stranded Watson strand-derived sequences and a library of single stranded Crick-strand derived sequences of the duplex sequencing library.

### Target Enrichment

In some embodiments of any one of the methods herein, amplicons produced by the initial amplification are enriched for one or more target polynucleotides. In some embodiments, prior to target enrichment, single-stranded DNA libraries are prepared from amplicons produced by the initial amplification. Exemplary methods for producing the single-stranded DNA libraries are described herein.

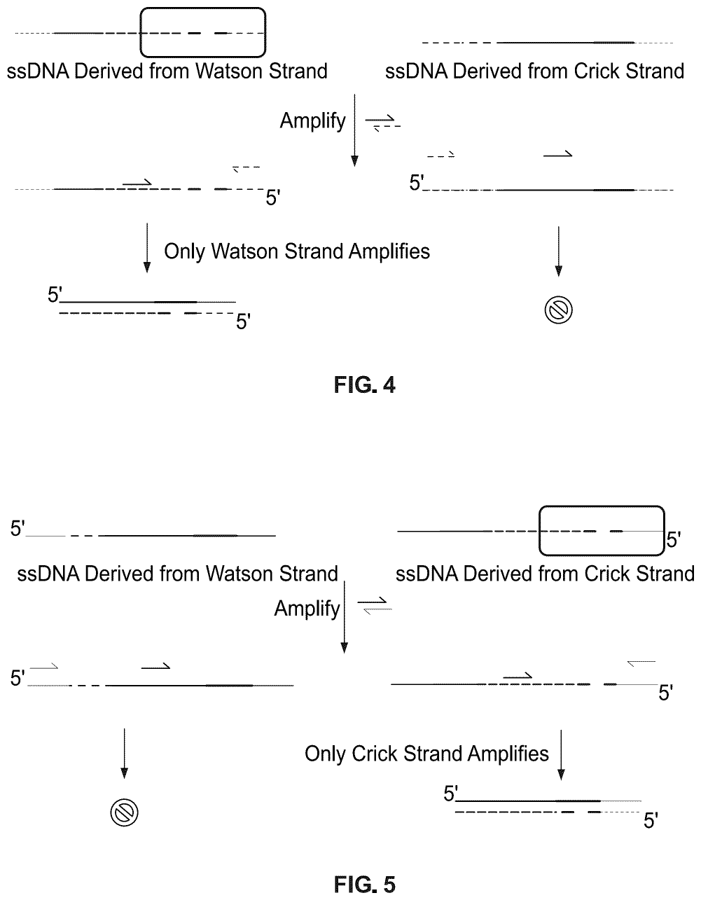

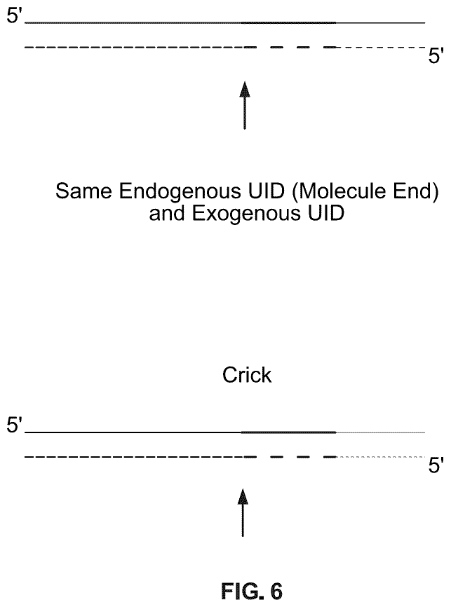

Any appropriate method can be used to amplify a target region from a library of amplification products (e.g., a duplex sequencing library, a library of single stranded Watson strand-derived sequences, or a library of single stranded Crick-strand derived sequences generated as described herein). In some cases, a target region can be amplified from library of amplification products by subjecting the library of amplification products to a PCR amplification using a primer pair where a primer (e.g., a first primer) that can target (e.g., target and bind to) an adapter sequence (e.g., an adapter sequence containing a molecular barcode) present in an amplification product generated as described herein (e.g., by ligating a 3′ duplex adapter including a first molecular barcode and a 5′ adapter including a second molecular barcode to a nucleic acid fragment in a duplex sequencing library prior to the amplification) and a primer (e.g., a second primer) that can target (e.g., target and bind to) a target region (e.g., a region of interest). In some cases, a library of single stranded Watson strand-derived sequences and a library of single stranded Crick-strand derived sequences can be generated as shown in FIG. 4 and FIG. 5. In some cases, a library of single stranded Watson strand-derived sequences and a library of single stranded Crick-strand derived sequences can be generated as described in Example 2.

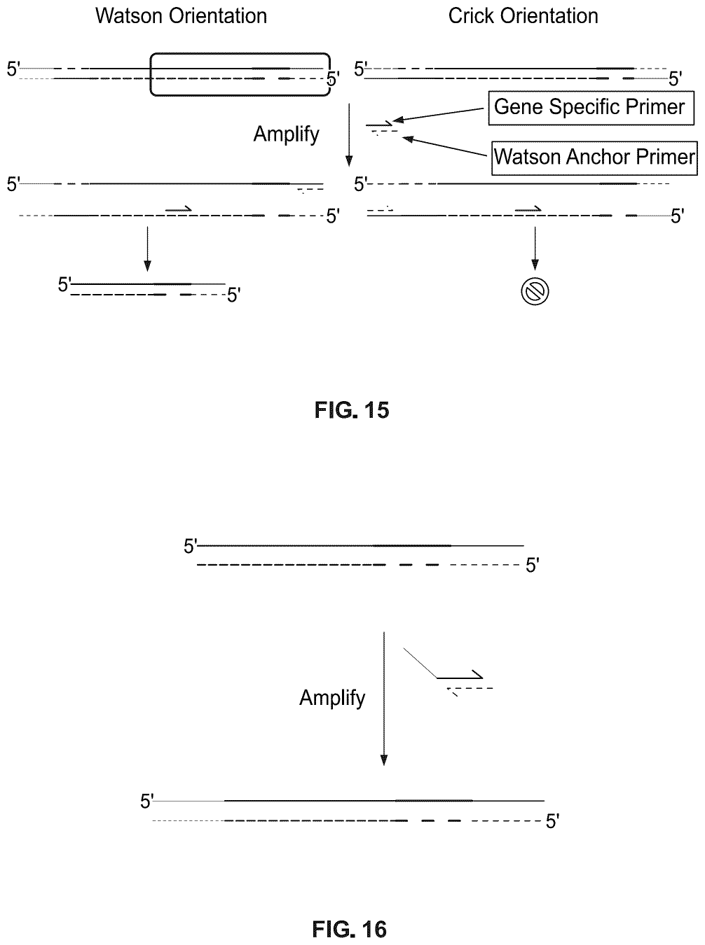

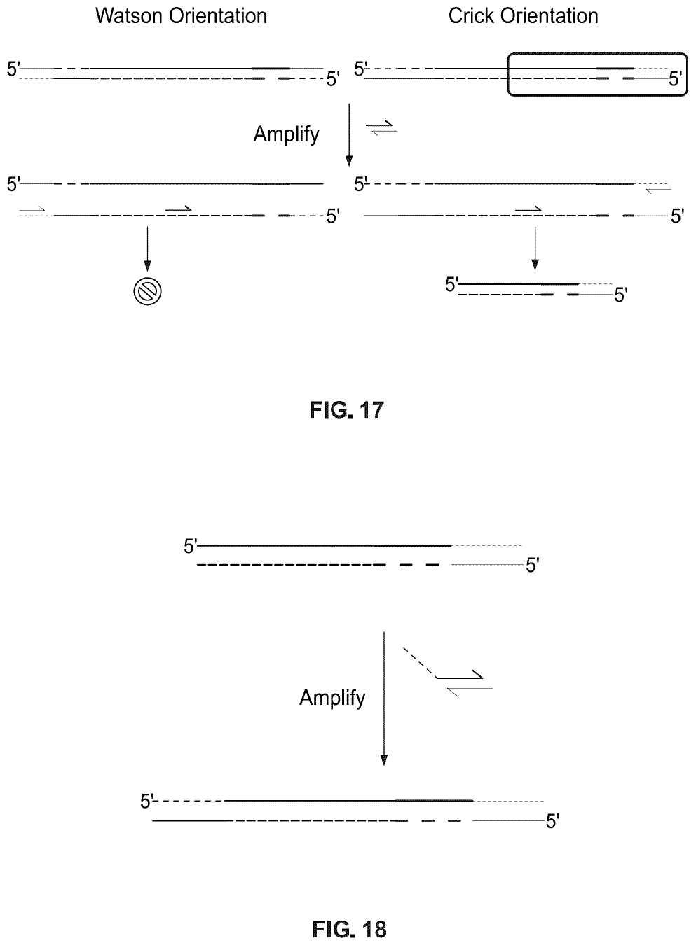

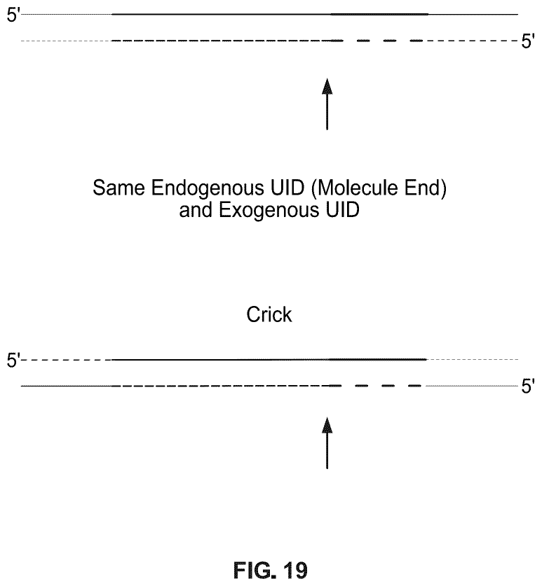

In some cases, a target region can be amplified from a library of amplification products (e.g., a duplex sequencing library, a library of single stranded Watson strand-derived sequences, or a library of single stranded Crick-strand derived sequences generated as described herein) in a single PCR amplification. For example, a target region can be amplified from a library of amplification products in a single PCR amplification using a primer pair including a first primer that can target an adapter sequence (e.g., an adapter sequence containing a molecular barcode) present in an amplification product generated as described herein (e.g., by ligating a 3′ duplex adapter including a first molecular barcode and a 5′ adapter including a second molecular barcode to a nucleic acid fragment in a duplex sequencing library prior to the amplification) and a second primer that can target a target region. For example, a target region can be amplified from a library of amplification products in a single PCR amplification as shown in FIG. 4, FIG. 5, FIG. 15, and FIG. 17.

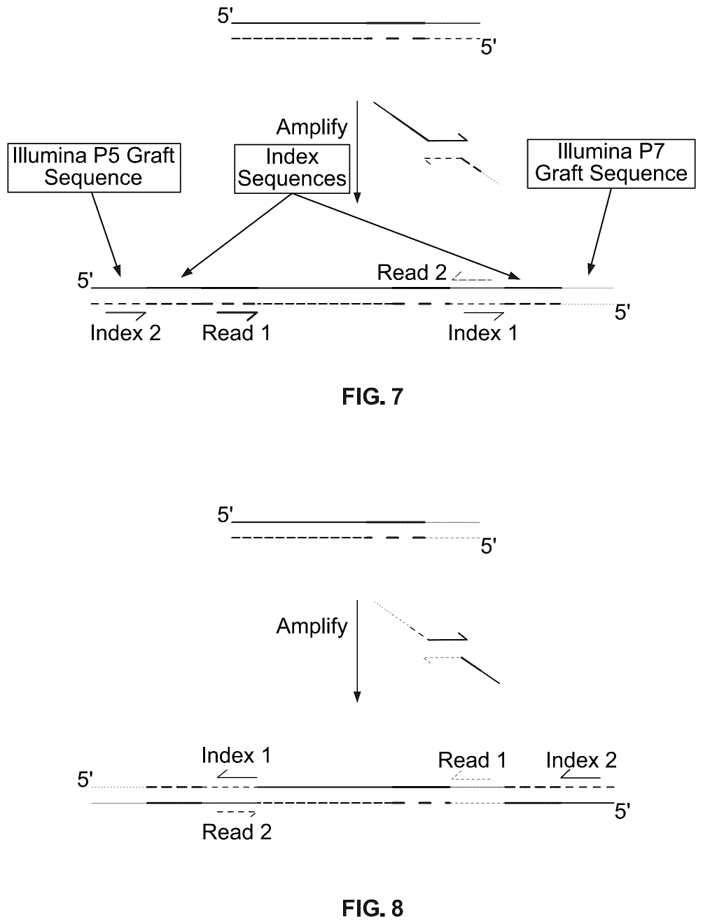

In some cases, a target region can be amplified from a library of amplification products (e.g., a duplex sequencing library, a library of single stranded Watson strand-derived sequences, or a library of single stranded Crick-strand derived sequences generated as described herein) in multiple PCR amplifications. Multiple PCR amplifications (e.g., a first PCR amplification and a subsequent, nested PCR amplification) can be used to increase the specificity of amplifying a target region. For example, a target region can be amplified from a library of amplification products in a series of PCR amplifications where a first PCR amplification uses a primer pair including a first primer that can target an adapter sequence (e.g., an adapter sequence containing a molecular barcode) present in an amplification product generated as described herein (e.g., by ligating a 3′ duplex adapter including a first molecular barcode and a 5′ adapter including a second molecular barcode to a nucleic acid fragment in a duplex sequencing library prior to the amplification) and a second primer that can target a target region, and subjecting the amplification products generated in the first PCR amplification to a subsequent, nested PCR amplification that uses a primer pair including a first primer that can target an adapter sequence (e.g., an adapter sequence containing a molecular barcode) present in an amplification product generated as described herein (e.g., by ligating a 3′ duplex adapter including a first molecular barcode and a 5′ adapter including a second molecular barcode to a nucleic acid fragment in a duplex sequencing library prior to the amplification) and a second primer that can target a nucleic acid sequence from the target region that is present in the amplification products generated in the first PCR amplification. For example, a target region can be amplified from a library of amplification products in a series of PCR amplifications as shown in FIG. 7, FIG. 8, FIG. 16, and FIG. 18.

Any appropriate primer pair can be used to amplify a target region from a library of amplification products (e.g., a duplex sequencing library, a library of single stranded Watson strand-derived sequences, or a library of single stranded Crick-strand derived sequences generated as described herein). A primer can include, without limitation, from about 12 nucleotides to about 30 nucleotides. In some cases, a primer pair can include a primer (e.g., a first primer) that can target (e.g., target and bind to) an adapter sequence (e.g., an adapter sequence containing a molecular barcode) present in an amplification product generated as described herein (e.g., by ligating a 3′ duplex adapter including a first molecular barcode and a 5′ adapter including a second molecular barcode to a nucleic acid fragment in a duplex sequencing library prior to the amplification) and a primer (e.g., a second primer) that can target (e.g., target and bind to) a target region (e.g., a region of interest). Examples of primers that can target an adapter sequence containing a molecular barcode present in an amplification product generated as described herein (e.g., by ligating a 3′ duplex adapter including a first molecular barcode and a 5′ adapter including a second molecular barcode to a nucleic acid fragment in a duplex sequencing library prior to the amplification) include, without limitation, an i5 index primer and an i7 index primer. Primers that can target a target region can include a sequence that is complementary to the target region. In cases where a target region is a nucleic acid encoding TP53, examples of primers that can target nucleic acid encoding TP53 include, without limitation, TP53_342_GSP1 and TP53_GSP2. In some cases, when a target region is a nucleic acid encoding TP53, a primer targeting the nucleic acid encoding TP53 can be as described in Example 2.

In some cases, one or both primers of a primer pair used to amplify a target region from a library of amplification products (e.g., a duplex sequencing library, a library of single stranded Watson strand-derived sequences, or a library of single stranded Crick-strand derived sequences generated as described herein) can include one or more molecular barcodes.

In some cases, one or both primers of a primer pair used to amplify a target region from a library of amplification products (e.g., a duplex sequencing library, a library of single stranded Watson strand-derived sequences, or a library of single stranded Crick-strand derived sequences generated as described herein) can include one or more graft sequences (e.g. graft sequences for next generation sequencing).

In an aspect, the target enrichment comprises (a) selectively amplifying amplicons of Watson strands comprising the target polynucleotide sequence with a first set of Watson target-selective primer pairs, the first set of Watson target-selective primer pairs comprising: (i) a first Watson target-selective primer comprising a sequence complementary to the R2 sequencing primer site of the universal 3′ adapter sequence, and (ii) a second Watson target-selective primer comprising a target-selective sequence, thereby creating target Watson amplification products; and (b) selectively amplifying amplicons of Crick strands comprising the same target polynucleotide sequence with a first set of Crick target-selective primer pairs, the first set of Crick target-selective primer pairs comprising: (i) a first Crick target-selective primer comprising a sequence complementary to the R1 sequencing primer site of the universal 5′ adapter sequence, and (ii) a second Crick target-selective primer comprising the same target-selective sequence as the second Watson target-selective primer sequence, thereby creating target Crick amplification products.

In some embodiments, the method further comprises purifying the target Watson amplification products and the target Crick amplification products from non-target polynucleotides. In some embodiments, the purifying comprises attaching the target Watson amplification products and the target Crick amplification products to a solid support. In some embodiments, the first Watson target-selective primer and first Crick target-selective primer comprises a first member of an affinity binding pair, and wherein the solid support comprises a second member of the affinity binding pair. In some embodiments, the first member is biotin and the second member is streptavidin. In some embodiments, the solid support comprises a bead, well, membrane, tube, column, plate, sepharose, magnetic bead, or chip. In some embodiments, the method comprises removing polynucleotides that are not attached to the solid support.

In some embodiments, the method further comprises (a) further amplifying the target Watson amplification products with a second set of Watson target-selective primers, the second set of Watson target-selective primers comprising (i) a third Watson target-selective primer comprising a sequence complementary to the R2 sequencing primer site of the universal 3′ adapter sequence, and (ii) a fourth Watson target-selective primer comprising, in the 5′ to 3′ direction, an R1 sequencing primer site and a target-selective sequence selective for the same target polynucleotide, thereby creating target Watson library members; (b) further amplifying the target Crick amplification products with a second set of Crick target-selective primers, the second set of Crick target-selective primers comprising (i) a third Crick target-selective primer comprising a sequence complementary to the R1 sequencing primer site of the universal 3′ adapter sequence, and (ii) a fourth Crick target-selective primer comprising, in the 5′ to 3′ direction, an R2 sequencing primer site and the target-selective sequence selective for the same target polynucleotide of the fourth Watson target-selective primer, thereby creating target Crick library members.

In some embodiments, the third Watson and Crick target-selective primers further comprise a sample barcode sequence. In some embodiments, the third Watson target-selective primer further comprises a first grafting sequence that enables hybridization to a first grafting primer on a sequencer and wherein the third Crick target-selective primer further comprises a second grafting sequence that enables hybridization to a second grafting primer on the sequencer. In some embodiments, the fourth Watson target-selective primer further comprises the second grafting sequence and wherein the fourth Crick target-selective primer further comprises the first grafting sequence. In some embodiments, the first grafting sequence is a P7 sequence and wherein the second grafting sequence is a P5 sequence.

Any appropriate PCR conditions can be used to generate an amplified target region as described herein (e.g., from a library of amplification products such as a duplex sequencing library, a library of single stranded Watson strand-derived sequences, or a library of single stranded Crick-strand derived sequences generated). Exemplary PCR conditions are described herein. PCR conditions used to generate an amplified target region as described herein (e.g., from a library of amplification products such as a duplex sequencing library, a library of single stranded Watson strand-derived sequences, or a library of single stranded Crick-strand derived sequences generated) can include any appropriate number of PCR amplification cycles. In some cases, PCR amplification can include, without limitation, from about 1 to about 50 cycles. For example, when PCR amplification of an amplified target region includes a single PCR amplification, the PCR amplification can include about 18 amplification cycles. For example, when PCR amplification of an amplified target region includes a first PCR amplification and a subsequent, nested PCR amplification, the first PCR amplification can include about 18 amplification cycles, and the subsequent, nested PCR amplification can include about 10 amplification cycles.

### Exemplary Targets

Any appropriate target region (e.g., a region of interest) can be amplified from a library of amplification products (e.g., a duplex sequencing library, a library of single stranded Watson strand-derived sequences, or a library of single stranded Crick-strand derived sequences generated as described herein) and assessed for the presence or absence of one or more mutations. In some cases, a target region can be a region of nucleic acid in which one or more mutations are associated with a disease or disorder. Examples of target regions that can be amplified and assessed for the presence or absence of one or more mutations include, without limitation, nucleic acid encoding tumor protein p53 (TP53), nucleic acid encoding breast cancer 1 (BRCA1), nucleic acid encoding BRCA2, nucleic acid encoding a phosphatase and tensin homolog (PTEN) polypeptide, nucleic acid encoding a AKT1 polypeptide, nucleic acid encoding a APC polypeptide, nucleic acid encoding a CDKN2A polypeptide, nucleic acid encoding a EGFR polypeptide, nucleic acid encoding a FBXW7 polypeptide, nucleic acid encoding a GNAS polypeptide, nucleic acid encoding a KRAS polypeptide, nucleic acid encoding a NRAS polypeptide, nucleic acid encoding a PIK3CA polypeptide, nucleic acid encoding a BRAF polypeptide, nucleic acid encoding a CTNNB1 polypeptide, nucleic acid encoding a FGFR2 polypeptide, nucleic acid encoding a HRAS polypeptide, and nucleic acid encoding a PPP2R1A polypeptide, In some cases, a target region that can be amplified and assessed for the presence or absence of one or more mutations can be nucleic acid encoding TP53. For example, nucleic acid encoding TP53 can be amplified and assessed as described in Example 2.

Any appropriate method can be used to assess a target region (e.g., an amplified target region) for the presence or absence of one or more mutations. In some cases, one or more sequencing methods can be used to assess an amplified target region for the presence or absence of one or more mutations.

### Sequence Determination

In some cases, one or more sequencing methods can be used to assess an amplified target region determine whether the mutation(s) are present on both the Watson strand and the Crick strand. In some cases, sequencing reads can be used to assess an amplified target region for the presence or absence of one or more mutations and can be used to determine whether the mutation(s) are present on both the Watson strand and the Crick strand. Examples of sequencing methods that can be used to assess an amplified target region for the presence or absence of one or more mutations as describe herein include, without limitation, single read sequencing, paired-end sequencing, NGS, and deep sequencing. In some embodiments, the single read sequencing comprises sequencing across the entire length of the templates to generate the sequence reads. In some embodiments, the sequencing comprises paired end sequencing. In some embodiments, the sequencing is performed with a massively parallel sequencer. In some embodiments, the massively parallel sequencer is configured to determine sequence reads from both ends of template polynucleotides.

### Analysis of Sequence Reads

In some embodiments, the sequence reads are mapped to a reference genome.

In some embodiments, the sequence reads are assigned into UID families. A UID family can comprise sequence reads from amplicons originating from an original template, e.g., original double-stranded DNA fragment from a nucleic acid sample.

In some embodiments, each member of a UID family comprises the same exogenous UID sequence. In some embodiments, each member of a UID family further comprises the same endogenous UID sequence. Endogenous UIDs are described herein.

In some embodiments, each member of a UID family further comprises the same exogenous UID sequence and the same endogenous UID sequence. In some embodiments, the combination of the exogenous UID sequence and endogenous UID sequence are unique to the UID family. In some embodiments, the combination of the exogenous UID sequence and endogenous UID sequence does not exist in another UID family represented in the nucleic acid sample.

The number of members of a UID family can depend on the depth of sequencing. In some embodiments, a UID family comprises at least 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, or 1000 members. In some embodiments, a UID family comprises about 2-1000 members, about 2-500 members, about 2-100 members, about 2-50 members, or about 2-20 members.

In some embodiments, the sequence reads of an individual UID family are assigned to a Watson subfamily and a Crick subfamily. In some embodiments, the sequence reads of an individual UID family are assigned to the Watson and Crick subfamilies based on the orientation of the insert relative to the adapter sequences. In some embodiments, the orientation of the insert relative to the adapter sequences is resolved by how the sequence reads were aligned as “read pairs” or “mate pairs”.

In some embodiments, the assignment of the sequence reads into the Watson and Crick subfamilies are based on spatial relationship of the exogenous UID sequence to the R1 and R2 read sequence. In some embodiments, members of the Watson subfamily are characterized by the exogenous UID sequence being downstream of the R2 sequence and upstream of the R1 sequence. In some embodiments, members of the Crick subfamily are characterized by the exogenous UID sequence being downstream of the R1 sequence and upstream of the R2 sequence. In some embodiments, members of the Watson subfamily are characterized by the exogenous UID sequence being in greater proximity to the R2 sequence and lesser proximity to the R1 sequence. In some embodiments, members of the Crick subfamily are characterized by the exogenous UID sequence being in greater proximity to the R1 sequence and in lesser proximity to the R2 sequence. In some embodiments, members of the Watson subfamily are characterized by the exogenous UID sequence being immediately downstream or within 1-70, 1-60, 1-50, 1-40, 1-30, 1-20, 1-10, or 1-5 nucleotides of the R2 sequence. In some embodiments, members of the Crick subfamily are characterized by the exogenous UID sequence being immediately downstream or within 1-70, 1-60, 1-50, 1-40, 1-30, 1-20, 1-10, or 1-5 nucleotides of the R1 sequence.

In some embodiments, a UID subfamily (e.g., Watson subfamily and/or Crick subfamily) comprises at least 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 members. In some embodiments, a UID subfamily (e.g., Watson subfamily and/or Crick subfamily) comprises about 2-500 members, about 2-100 members, about 2-50 members, about 2-20 members, or about 2-10 members.

In some embodiments, a nucleotide sequence is determined to accurately represent a Watson strand of an analyte DNA fragment, e.g., a double stranded DNA fragment from the nucleic acid sample, when a threshold percentage (or a percentage exceeding a threshold) of members of the Watson subfamily contain the sequence. In some embodiments, a nucleotide sequence is determined to accurately represent a Crick strand of an analyte DNA fragment, e.g., a double stranded DNA fragment from the nucleic acid sample, when a threshold percentage (or a percentage exceeding a threshold) of members of the Crick subfamily contain the sequence.

Thresholds can be determined by a skilled artisan based on, e.g., number of the members of the subfamily, the particular purpose of the sequencing experiment, and the particular parameters of the sequencing experiment. In some embodiments, the threshold is set at 1%, 5%, 10%, 20%, 30%, 40%, 50%, 60%, 70%, 75%, 80%, 85%, 90%, 95%, 96%, 97%, 98%, 99%, or 100%. In particular embodiments, the threshold is set at 50%. By way of example only, in an embodiment wherein the threshold is set at 50%, a nucleotide sequence is determined to accurately represent a Watson or Crick strand of an analyte DNA fragment, e.g., a double stranded DNA fragment from the nucleic acid sample, when at least 50% of the subfamily members contain the sequence. By way of other example only, in an embodiment wherein the threshold is set at 50%, a nucleotide sequence is determined to accurately represent a Watson or Crick strand of an analyte DNA fragment, e.g., a double stranded DNA fragment from the nucleic acid sample, when more than 50% of the subfamily members contain the sequence.

In some embodiments, the sequence accurately representing the Watson strand of the analyte DNA fragment is determined to have a mutation. In some embodiments, the sequence accurately representing the Watson strand of the analyte DNA fragment is determined to have a mutation when the sequence differs from a reference sequence that lacks the mutation.

In some embodiments, the sequence accurately representing the Crick strand of the analyte DNA fragment is determined to have a mutation. In some embodiments, the sequence accurately representing the Crick strand of the analyte DNA fragment is determined to have a mutation when the sequence differs from a reference sequence that lacks the mutation.

In some embodiments, the analyte DNA fragment is determined to have the mutation when sequence accurately representing the Watson strand the sequence accurately representing the Crick strand comprise the same mutation.

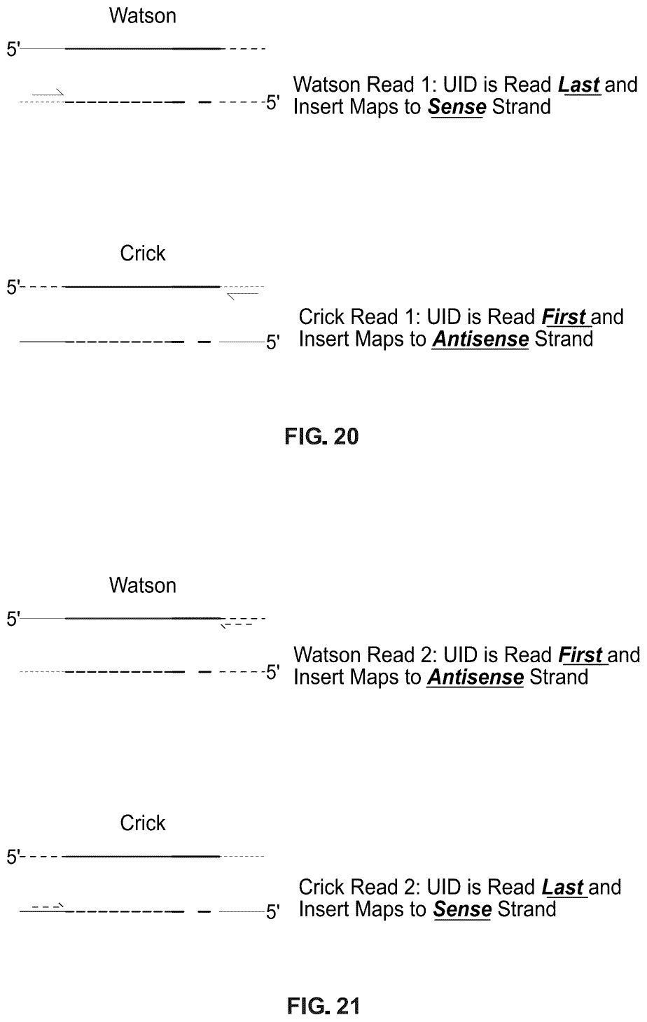

In some cases, the location of the molecular barcode within the paired-end sequencing reads of the amplified target region can be used to distinguish which strand of the double stranded nucleic acid template the amplified target region was derived from. For example, when a first a paired-end sequencing read of an amplified target region indicates that a molecular barcode is read last, the amplified target region can be identified as being derived from the sense strand of the nucleic acid template, and when a first a paired-end sequencing read of an amplified target region indicates that a molecular barcode is read first, the amplified target region can be identified as being derived from the anti-sense strand of the nucleic acid template. For example, when a second a paired-end sequencing read of an amplified target region indicates that a molecular barcode is read first, the amplified target region can be identified as being derived from the anti-sense strand of the nucleic acid template, and when a second a paired-end sequencing read of an amplified target region indicates that a molecular barcode is read last, the amplified target region can be identified as being derived from the sense strand of the nucleic acid template. In some cases, paired-end sequencing can be used to distinguish amplification products derived from the Watson strand from amplification products derived from the Crick strand as shown in FIG. 20 and FIG. 21.

Following sequencing of target regions (e.g., target regions amplified as described herein), sequencing reads can be aligned to a reference genome and grouped by the molecular barcode present in each sequencing read. In some cases, sequencing reads that include the same molecular barcode and map to both the Watson strand and the Crick strand of the double stranded nucleic acid template (e.g., both the Watson strand and the Crick strand of the target region) can be identified as having duplex support. For example, when sequencing reads indicate the presence of one or more mutations in a target region include the same molecular barcode and map to both the Watson strand and the Crick strand of the target region, the mutation(s) can be identified as having duplex support.

### Kits

Also provided herein are kits. The kits may comprise sets of primer pairs for amplification of one or more target polynucleotides.

In some embodiments, the kit comprises (a) a first set of Watson target-selective primer pairs, comprising (i) one or more first Watson target-selective primers comprising a sequence complementary to the R2 sequencing primer site of the universal 3′ adapter sequence, and (ii) one or more second Watson target-selective primers, each of the one or more second Watson target-selective primers comprising a target-selective sequence; (b) a first set of Crick target-selective primer pairs, comprising (i) one or more Crick target-selective primers comprising a sequence complementary to the R1 sequencing primer site of the universal 5′ adapter sequence, and (ii) one or more second Crick target-selective primers, each of the one or more second Crick target-selective primers comprising the same target-selective sequence as the second Watson target-selective primer sequence; (c) a second set of Watson target-selective primer pairs, comprising (i) one or more third Watson target-selective primers comprising a sequence complementary to the R2 sequencing primer site of the universal 3′ adapter sequence, and (ii) one or more fourth Watson target-selective primers, each of the one or more fourth Watson target-selective primers comprising, in the 5′ to 3′ direction, an R1 sequencing primer site and a target-selective sequence selective for the same target polynucleotide; and (d) a second set of Crick target-selective primers comprising (i) one or more third Crick target-selective primers comprising a sequence complementary to the R1 sequencing primer site of the universal 3′ adapter sequence, and (ii) one or more fourth Crick target-selective primers, each of the one or more fourth Crick target-selective primers comprising, in the 5′ to 3′ direction, an R2 sequencing primer site and a target-selective sequence selective for the same target polynucleotide.

The kit may comprise sets of primer pairs for multiplex amplification of a plurality of target polynucleotides.

### Computer Readable Medium

Also provided herein is a computer readable medium comprising computer executable instructions configured to implement any of the methods described herein. The computer readable medium can comprise computer executable instructions for analyzing sequence data from a nucleic acid sample, wherein the data is generated by a method of any one of the preceding claims.

The computer readable medium can implement a method for semi-automated or automated sequence data analysis.

In some embodiments, the computer readable medium comprises executable instructions for (a) assigning sequence reads into UID families, wherein each member of a UID family comprises the same exogenous UID sequence; (b) assigning sequence reads of each UID family into Watson and Crick subfamilies; (c) identifying a nucleotide sequence as accurately representing a Watson strand of an analyte DNA fragment when a threshold percentage of members of the Watson subfamily contain the sequence; (d) identifying a nucleotide sequence as accurately representing a Crick strand of an analyte DNA fragment when a threshold percentage of members of the Crick subfamily contain the sequence; (e) identifying a mutation in the nucleotide sequence accurately representing the Watson Strand when the sequence accurately representing the Watson Strand differs from a reference sequence that lacks the mutation; (f) identifying a mutation in the nucleotide sequence accurately representing the Crick Strand when the sequence accurately representing the Crick Strand differs from a reference sequence that lacks the mutation; and (g) identifying a mutation in the analyte DNA fragment when the mutation in the nucleotide sequence accurately representing the Watson strand and the mutation in the nucleotide sequence accurately representing the Crick strand are the same mutation.

In some embodiments, the computer readable medium comprises executable code for assigning UID family members to the Watson subfamily or Crick subfamily based on spatial relationship of the exogenous UID sequence to the R1 and R2 read sequence. In some embodiments, the computer executable code assigns UID family members to the Watson subfamily when the exogenous UID sequence is downstream of the R2 sequence and upstream of the R1 sequence. In some embodiments, the computer executable code assigns UID family members to the Crick subfamily when the exogenous UID sequence is downstream of the R1 sequence and upstream of the R2 sequence. In some embodiments, the computer executable code assigns UID family members to the Watson subfamily when the exogenous UID sequence is in greater proximity to the R2 sequence and lesser proximity to the R1 sequence. In some embodiments, the computer executable code assigns UID family members to the Crick subfamily when the exogenous UID sequence is in greater proximity to the R1 sequence and in lesser proximity to the R2 sequence. In some embodiments, the computer executable code assigns UID family members to the Watson subfamily when the exogenous UID sequence is immediately downstream or within 1-70, 1-60, 1-50, 1-40, 1-30, 1-20, 1-10, or 1-5 nucleotides of the R2 sequence. In some embodiments, the computer executable code assigns UID family members to the Crick subfamily when the exogenous UID sequence is immediately downstream or within 1-70, 1-60, 1-50, 1-40, 1-30, 1-20, 1-10, or 1-5 nucleotides of the R1 sequence.

In some embodiments, the computer readable medium comprises executable code for mapping the sequence reads to a reference genome. In some embodiments, the reference genome is a human reference genome.

In some embodiments, the computer readable medium comprises executable code for generating a report of disease status, prognosis, or theranosis based on the presence, absence, or amount of a mutation in the sample. In some embodiments, the disease is cancer.

In some embodiments, the computer readable medium comprises executable code for generating a report of therapeutic options based on the presence, absence, or amount of a mutation in the sample.

In some embodiments, the computer readable medium comprises executable code for transmission of the data over a network.

### Computer System

Also provided herein are computer systems. In some embodiments, the computer system comprises a memory unit configured to receive and store sequence data from a nucleic acid sample, the data generated by a method described herein; and a processor communicatively coupled to the storage memory, wherein the processor comprises a computer readable medium disclosed herein.

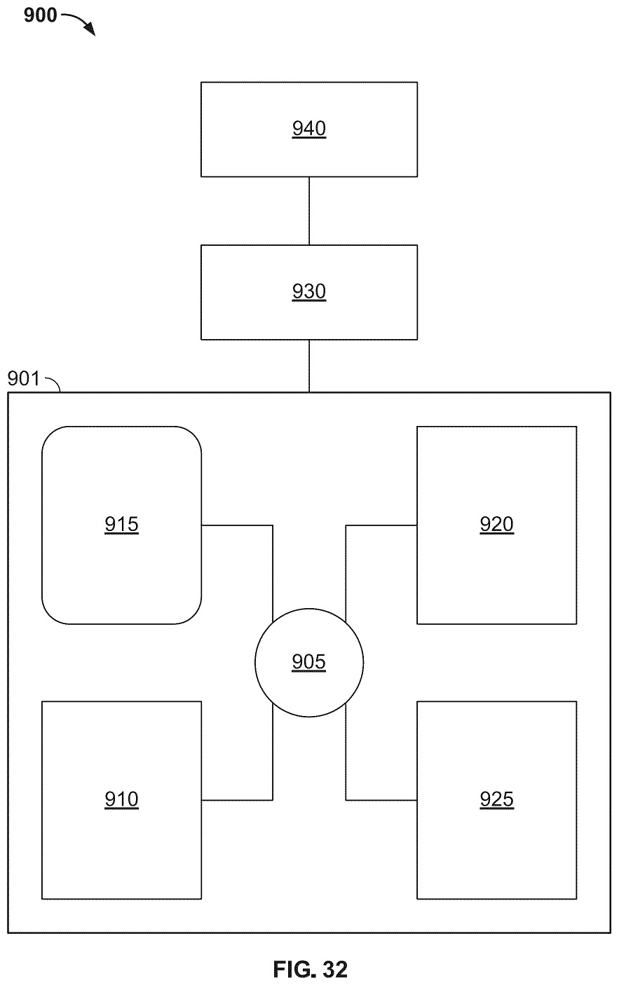

FIG. 32 depicts an exemplary computer system 900 adapted to enable a user to analyze a nucleic acid sample according to any of the methods described herein. The system 900 includes a central computer server 901 that is programmed to implement exemplary methods described herein. The server 901 includes a central processing unit (CPU, also “processor”) 905 which can be a single core processor, a multi core processor, or plurality of processors for parallel processing. The server 901 also includes memory 910 (e.g. random access memory, read-only memory, flash memory); electronic storage unit 915 (e.g. hard disk); communications interface 920 (e.g. network adapter) for communicating with one or more other systems, e.g., a sequencing system; and peripheral devices 925 which may include cache, other memory, data storage, and/or electronic display adapters. The memory 910, storage unit 915, interface 920, and peripheral devices 925 are in communication with the processor 905 through a communications bus (solid lines), such as a motherboard. The storage unit 915 can be a data storage unit for storing data. The server 901 is operatively coupled to a computer network (“network”) 930 with the aid of the communications interface 920. The network 930 can be the Internet, an intranet and/or an extranet, an intranet and/or extranet that is in communication with the Internet, a telecommunication or data network. The network 930 in some cases, with the aid of the server 901, can implement a peer-to-peer network, which may enable devices coupled to the server 901 to behave as a client or a server.

The storage unit 915 can store files, such as sequence data, barcode sequence data, or any aspect of data associated with the invention. The data storage unit 915 may be coupled with data relating to locations of cells in a virtual grid.

The server can communicate with one or more remote computer systems through the network 930. The one or more remote computer systems may be, for example, personal computers, laptops, tablets, telephones, Smart phones, or personal digital assistants.

In some situations the system 900 includes a single server 901. In other situations, the system includes multiple servers in communication with one another through an intranet, extranet and/or the Internet.

The server 901 can be adapted to store sequence data, data regarding the nucleic acid sample, data regarding the biological sample, data regarding the subject, and/or other information of potential relevance. Such information can be stored on the storage unit 915 or the server 901 and such data can be transmitted through a network.

Methods as described herein can be implemented by way of machine (e.g., computer processor) computer readable medium (or software) stored on an electronic storage location of the server 901, such as, for example, on the memory 910, or electronic storage unit 915. During use, the code can be executed by the processor 905.

In some cases, the code can be retrieved from the storage unit 915 and stored on the memory 910 for ready access by the processor 905. In some situations, the electronic storage unit 915 can be precluded, and machine-executable instructions are stored on memory 910. Alternatively, the code can be executed on a second computer system 940.

Aspects of the systems and methods provided herein, such as the server 901, can be embodied in programming. Various aspects of the technology may be thought of as “products” or “articles of manufacture” typically in the form of machine (or processor) executable code and/or associated data that is carried on or embodied in a type of machine readable medium (e.g., computer readable medium). Machine-executable code can be stored on an electronic storage unit, such memory (e.g., read-only memory, random access memory, and flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless likes, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, tangible storage medium, a carrier wave medium, or physical transmission medium. Non-volatile storage media can include, for example, optical or magnetic disks, such as any of the storage devices in any computer(s) or the like, such may be used to implement the system. Tangible transmission media can include: coaxial cables, copper wires, and fiber optics (including the wires that comprise a bus within a computer system). Carrier-wave transmission media may take the form of electric or electromagnetic signals, or acoustic or light waves such as those generated during radio frequency (RF) and infrared (IR) data communications. Common forms of computer-readable media therefore include, for example: a floppy disk, a flexible disk, hard disk, magnetic tape, any other magnetic medium, a CD-ROM, DVD, DVD-ROM, any other optical medium, punch cards, paper tame, any other physical storage medium with patterns of holes, a RAM, a ROM, a PROM and EPROM, a FLASH-EPROM, any other memory chip or cartridge, a carrier wave transporting data or instructions, cables, or links transporting such carrier wave, or any other medium from which a computer may read programming code and/or data. Many of these forms of computer readable media may be involved in carrying one or more sequences of one or more instructions to a processor for execution.

The results of the analysis can be presented to a user with the aid of a user interface, such as a graphical user interface.

The invention will be further described in the following examples, which do not limit the scope of the invention described in the claims.

### EXAMPLES

### Example 1: Duplex Anchored PCR

### Materials and Methods

### Duplex Anchored PCR Library Preparation

This protocol can prepare duplex libraries using a Swift Accel-NGS 2S PCR-Free Library Kit (Cat. #20024 and 20096) and specific truncated adapters and primers. In some cases, full-length P5 and P7 graft sequences can be added to the library by a separate PCR for sequencing on an Illumina instrument.

This protocol is for PCR tubes but can be scaled to PCR plates.

### Materials:

1. Swift Accel-NGS 2S PCR-Free Library Kit (Cat. #20024 and 20096)
2. 3′ Swift N14 Adapter 1 v3A
   a.

(SEQ ID NO: 1)
/5Phos/ATAAAACGACGGCNNNNNNNNNNNNNNAGATCGGAAG
AGCACACGTCTGAACTCCAG*T*C/3SpC3/

b. Order PAGE purified from IDT, 1 μmole synthesis scale, lyophilized
c. /3SpC3/ can be substituted with /3Phos/, phosphorothioate linkages are not required, and oligo may be HPLC purified
3. 3′ Swift Adapter 2 v3′dT

a.
(SEQ ID NO: 3)
GCCGUCGUUUUA/33dT/

b. /33dT/is IDT off-catalog modification for 3′-deoxyT
c. Order PAGE purified from IDT, 1 μmole synthesis scale, lyophilized
4. 5′ Swift Adapter

a.
(SEQ ID NO: 4)
/5SpC3/A*C*ACTCTTTCCCTACACGACGCTCTTCCGATCT

b. Order PAGE purified from IDT, 1 μmole synthesis scale, lyophilized
c. /5SpC3/ and phosphorothioate linkages are not required, and oligo should be HPLC purified
d. Can substitute with truB2 reagent from 2S Dual Indexing Kit (Cat. No. 28096)
5. NEB Ultra II Q5 Master Mix (Cat. No. M0544L)
6. Truncated P5 Primer

a.
(SEQ ID NO: 4)
ACACTCTTTCCCTACACGACGCTCTTCCGATCT

b. No modifications required, desalted from IDT, 100 μM in IDTE
7. Truncated P7 Primer

a.
(SEQ ID NO: 5)
GACTGGAGTTCAGACGTGTGCTCTTCCGATCT

b. No modifications required, desalted from IDT, 100 μM in IDTE
8. SPRIselect beads (Beckman Coulter, Cat. No. B23317/B23318/B23319)
9. 80% EtOH (˜2 mL per sample)
10. PCR tube strips (e.g., GeneMate VWR Cat. No. 490003-710)
11. Magnetic rack (e.g., Permagen MSRLV08)
12. USER Enzyme (NEB Cat. No. M5505L)—which is a mixture of uracil-DNA glycosylase and DNA glycosylase-lyase Endonuclease VIII.

### Prepare Custom Adapters (can be Done Once for a Large Batch):

1. If not using Swift's truB2 reagent, resuspend 5′ Swift Adapter to 42 μM in Low EDTA TE (included with Swift 2S kit)
2. Resuspend 3′ Swift N14 Adapter 1 v3A to 100 μM in Low EDTA TE (included with Swift 2S kit). Store at −20° C. for subsequent uses.
3. Resuspend 3′ Swift Adapter 2 v3′dT to 100 μM in Low EDTA TE (included with Swift 2S kit). Store at −20° C. for subsequent uses.
4. Anneal 3′ Swift N14 Adapter 1 v3A to 3′ Swift Adapter 2 v3′dT by mixing 100 μl of each oligo at room temperature. Label tube as 3′ Swift N14 v3′dT Duplex Adapter, 50 μM. Final concentration of the 3′ duplex adapter will be 50 μM. Incubate for at least 5 minutes at room temperature before use. Store at −20 C for subsequent uses.

### Technical Notes:

Enzyme tubes can be removed from −20° C. storage and placed on ice, for about 10 minutes to allow enzymes to reach 4° C. prior to pipetting. Pipetting enzymes at −20° C. may result in a shortage of enzyme reagents.

After thawing reagents to 4° C., reagents (except the enzymes) can be briefly vortexed to mix them well. Spin all tubes in a microfuge to collect contents prior to opening.

Assemble all reagent master mixes on ice and scale volumes as appropriate, using 5% excess volume to compensate for pipetting loss.

Reagents should be added to the master mix in the specified order as stated throughout the protocol.

Reagents can be prepared in advance (e.g., to ensure that the magnetic beads do not dry out during size selection steps).

### Step 1: Template Repair

1. Transfer 11 ng of cfDNA sample to a 0.2 mL PCR tube and adjust the volume of the sample to a final volume of 37 μl using Low EDTA TE, if necessary.
2. Add 3 μl USER Enzyme to each sample.
3. Mix by vortexing, and gently spin down to collect all liquid to the bottom of the tube.
4. Place the samples in the thermocycler, programmed at 37° C. for 15 minutes with lid heating off.

### Step 2: End Repair 1

1. Gently spin down samples to collect any condensation.
2. Add 20 μl of pre-mixed Repair I Master Mix (see Table 1) to each sample containing the 40 μl DNA sample.

TABLE 1
End Repair I Master Mix

| Reagent | Volume per Sample |
|---|---|
| Low EDTA TE | 13 μl |
| Buffer W1 | 6 μl |
| Enzyme W2 | 1 μl |
| Total Volume | 20 μl |

3. Mix by vortexing, gently spin down, place in the thermocycler, and run the Repair I Thermocycler Program in the order described below.
   a. 37° C., 5 minutes, lid heating on (lid set at 75° C.)
   b. 65° C., 2 minutes, lid heating on (lid set at 75° C.)
   c. 37° C., 5 minutes, lid heating on (lid set at 75° C.)
4. After thermocycler program completes, gently spin down tubes to collect condensation.
5. Clean up the Repair I reaction by adding 120 μl (2.0×) of SPRIselect beads. Mix by vortexing. Gently spin down to collect beads and incubate at room temperature for 5 minutes.
6. Collect beads by placing sample on magnetic rack for 5 minutes.
7. Remove and discard the supernatant without disturbing the pellet.
8. Add 180 μl of freshly prepared 80% ethanol solution to the sample while it is still on the magnetic rack. Use care not to disturb the pellet. Incubate for 30 seconds and then carefully remove the ethanol solution with P20 pipette.
9. Repeat above step for second wash with the 80% ethanol solution.
10. Remove any residual ethanol solution with P20 pipette, let beads dry for ˜30 seconds. Take care to not over dry beads, and immediately proceed to step 1 of End Repair 2.

### Step 3: End Repair 2

1. Add 50 μl of pre-mixed Repair II Master Mix (see Table 2) to the beads for each sample and mix by vortexing until homogeneous.

TABLE 2
End Repair II Master Mix

| Reagent | Volume per Sample |
|---|---|
| Low EDTA TE | 30 μl |
| Buffer G1 | 5 μl |
| Reagent G2 | 13 μl |
| Enzyme G3 | 1 μl |
| Enzyme G4 | 1 μl |
| Total Volume | 50 μl |

2. Place the samples in the thermocycler, programmed at 20° C. for 20 minutes with lid heating off.
3. After thermocycler program completes, gently spin down tubes to collect condensation.
4. Clean up the Repair 2 reaction by adding 90 μl (1.8×) of PEG/NaCl Solution. Mix by vortexing. Gently spin down to collect beads and incubate at room temperature for 5 minutes.
5. Collect beads by placing sample on magnetic rack for 5 minutes.
6. Remove and discard the supernatant without disturbing the pellet.
7. Add 180 μl of freshly prepared 80% ethanol solution to the sample while it is still on the magnetic rack. Use care not to disturb the pellet. Incubate for 30 seconds and then carefully remove the ethanol solution with P20 pipette.
8. Repeat above step for second wash with the 80% ethanol solution.
9. Remove any residual ethanol solution with P20 pipette, let beads dry for ˜30 seconds. Take care to not overdry beads, and immediately proceed to step 1 of Ligation 1.

### Step 4: Ligation 1

1. Add 30 μl of pre-mixed Ligation I Master Mix (see Table 3) to the beads for each sample and mix by vortexing until homogeneous.

TABLE 3
Ligation I Master Mix

| Reagent | Volume per Sample |
|---|---|
| Low EDTA TE | 20 μl |
| 3′ Swift N14 v3′dT Duplex Adapter, 50 uM | 5 μl |
| Buffer Y1 | 3 μl |
| Enzyme Y3 | 2 μl |
| Total Volume | 30 μl |

2. Place the samples in the thermocycler, programmed at 25° C. for 15 minutes with lid heating off.
3. After thermocycler program completes, gently spin down tubes to collect condensation.
4. Clean up the Ligation 1 reaction by adding 36 μl (1.2×) of PEG/NaCl Solution. Mix by vortexing. Gently spin down to collect beads and incubate at room temperature for 5 minutes.
5. Collect beads by placing sample on magnetic rack for 5 minutes.
6. Remove and discard the supernatant without disturbing the pellet.
7. Add 180 μl of freshly prepared 80% ethanol solution to the sample while it is still on the magnetic rack. Use care not to disturb the pellet. Incubate for 30 seconds and then carefully remove the ethanol solution with P20 pipette.
8. Repeat above step for second wash with the 80% ethanol solution.
9. Remove any residual ethanol solution with P20 pipette, let beads dry for ˜30 seconds. Take care to not overdry beads, and immediately proceed to step 1 of Ligation 2.

### Step 5: Ligation 2

1. Add 50 μl of pre-mixed Ligation II Master Mix (see Table 4) to the beads for each sample and mix by vortexing until homogeneous.

TABLE 4
Ligation II Master Mix

| Reagent | Volume per Sample |
|---|---|
| Low EDTA TE | 30 μl |
| Reagent B1 | 5 μl |
| 5′ Swift Adapter, 42 μM | 2 μl |
| OR | |
| Reagent truB2 | |
| Reagent B3 | 9 μl |
| Enzyme B4 | 1 μl |
| Enzyme B5 | 2 μl |
| Enzyme B6 | 1 μl |
| Total Volume | 50 μl |

2. Place the samples in the thermocycler, programmed at 40° C. for 10 minutes with lid heating off.
3. After thermocycler program completes, gently spin down tubes to collect condensation.
4. Clean up the Ligation 1 reaction by adding 52.5 μl (1.05×) of PEG/NaCl Solution. Mix by vortexing. Gently spin down to collect beads and incubate at room temperature for 5 minutes.
5. Collect beads by placing sample on magnetic rack for 5 minutes.
6. Remove and discard the supernatant without disturbing the pellet.
7. Add 180 μl of freshly prepared 80% ethanol solution to the sample while it is still on the magnetic rack. Use care not to disturb the pellet. Incubate for 30 seconds and then carefully remove the ethanol solution with P20 pipette.
8. Repeat above step for second wash with the 80% ethanol solution.
9. Remove any residual ethanol solution with P20 pipette, let beads dry for ˜30 seconds. Take care to not overdry beads, and immediately resuspend the beads in 24 μl of Low EDTA TE. Mix by vortexing and incubate for 2 minutes.
10. Gently spin down to collect beads and collect beads on magnetic rack for 2 minutes.

### Step 6: PCR-Library Amplification

1. Add 26 μl of pre-mixed PCR-Library Amplification Master Mix (see Table 5) to a clean tube for each sample.

TABLE 5
PCR-library Amplification Master Mix

| Reagent | Volume per Sample |
|---|---|
| NEB Ultra II Q5 Master Mix (2x) | 25 μl |
| Truncated P5 Primer, 100 μM | 0.5 μl |
| Truncated P7 Primer, 100 μM | 0.5 μl |
| Total Volume | 26 μl |

2. Carefully transfer the supernatant containing the final post-ligation library to the PCR-Library Amplification Master Mix.
3. Transfer any residual final post-ligation library using a P20 pipette. Take care to transfer as much as the supernatant as possible.
4. Mix by vortexing, gently spin down, place in the thermocycler, and run the PCR-Library Amplification Thermocycler Program in the order described below.

TABLE 6
Exemplary PCR-library Amplification Thermocycler Program

| Step | Temperature | Time | |
|---|---|---|---|
| Initial denaturation | 98° C. | 30 | seconds |
| 5 PCR Cycles | 98° C. | 10 | seconds |
| (4 GOTO Steps) | 65° C. | 75 | seconds |
| Final Extension | 65° C. | 5 | minutes |

| | | |
|---|---|---|
| Hold | 4° C. | Hold |

5. Clean up the PCR-Library Amplification reaction by adding 90 μl (1.8×) of SPRIselect beads. Mix by vortexing. Gently spin down to collect beads and incubate at room temperature for 5 minutes.
6. Collect beads by placing sample on magnetic rack for 5 minutes.
7. Remove and discard the supernatant without disturbing the pellet.
8. Add 180 μl of freshly prepared 80% ethanol solution to the sample while it is still on the magnetic rack. Use care not to disturb the pellet. Incubate for 30 seconds and then carefully remove the ethanol solution with P20 pipette.
9. Repeat above step for second wash with the 80% ethanol solution.
10. Remove any residual ethanol solution with P20 pipette, let beads dry for ˜30 seconds. Take care to not overdry beads, and immediately resuspend the beads in 47 μl of Low EDTA TE. Mix by vortexing and incubate for 2 minutes.
11. Gently spin down to collect beads and collect beads on magnetic rack for 2 minutes.
12. Carefully transfer the supernatant containing the final PCR-amplified library to a clean tube without carrying any beads.
13. Analyze 1 μl of the amplified libraries by TapeStation. Prominent peak should be present at ˜300 bp corresponding to adapter-ligated mononucleosomal DNA (180 bp+60 bp +59 bp).
14. Store libraries at −20° C.

### Accurate and Efficient Detection of Rare Mutations Using Duplex Anchored PCR

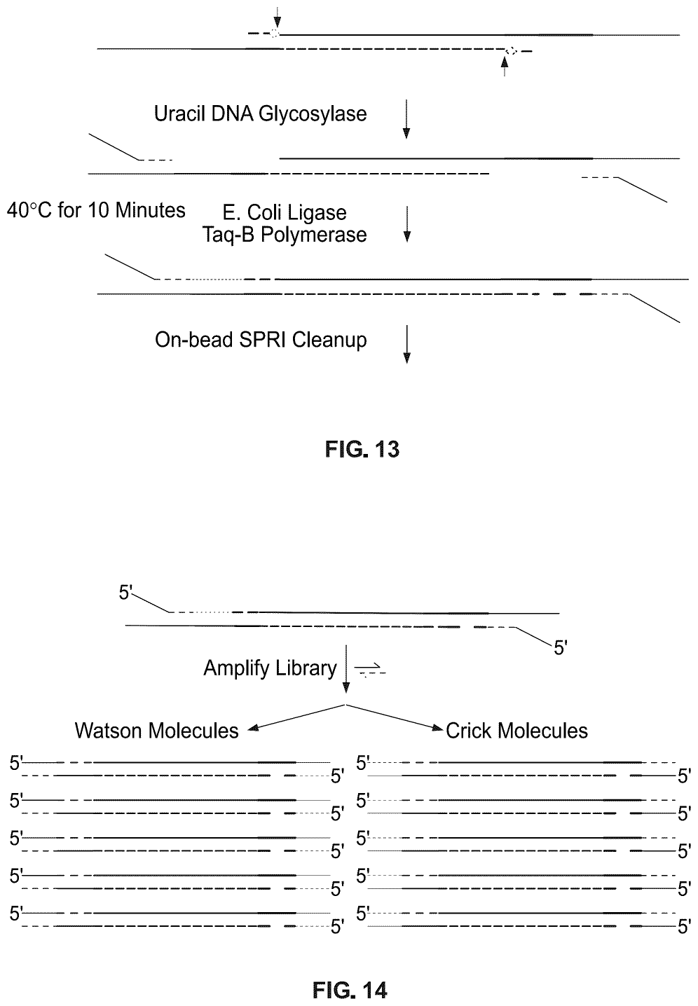

Sequencing libraries incorporating duplex molecular barcodes were generated by sequentially ligating two adapter molecules to double stranded input DNA. First, input DNA was end-repaired via blunting and dephosphorylation reactions (FIGS. 9 and 10). Following end-repair, a 3′ adapter containing a 5′ phosphate (3′ oligo #1) annealed to a short oligonucleotide with a blocked 3′ group (3′ oligo #2) was ligated to each 3′ end of the input DNA (FIG. 12). As one of the oligonucleotides contains a 3′ blocking group, only the oligonucleotide containing the 5′ phosphate (3′ oligo #1) was covalently attached to the input DNA at the 3′ ends. The attached 3′ oligonucleotide also contains a molecular barcode which uniquely labels each strand (FIG. 11). Next, the 3′ oligo containing a 3′ blocking group was degraded and a 5′ adapter oligo was ligated to each 5′ end via a nick translation-like reaction. Specifically, the 5′ adapter oligo anneals immediately upstream of the molecular barcode on 3′ adapter oligo #1 leaving a gap. This gap was filled in and sealed during a nick-translation-like reaction thereby generating a duplex molecular barcode in-situ on each end of the DNA fragments (FIG. 13). The resulting ligated products were purified and amplified via an initial whole-genome PCR (FIG. 14).

Following the initial whole-genome PCR, the product can optionally be purified and single stranded (ss) NA libraries corresponding to sense and anti-sense strands are generated (FIGS. 2 and 3).

The amplified DNA libraries were enriched for the desired targets using a strand-specific anchored PCR approach. This PCR enrichment utilized a single primer targeting the desired region of interest and a second primer targeting the ligated adapter sequence (FIGS. 4, 5, 15, 17). To increase the specificity of target enrichment, a second nested PCR can be performed using a single primer targeting the desired region of interest and a second primer targeting the ligated adapter sequence (FIGS. 7, 8, 16, 18). To increase the specificity of target enrichment, a second nested PCR can be used to incorporate sample barcodes as well as the requisite graft sequences necessary for next generation sequencing. The resulting libraries are then quantified, normalized, and sequenced.

Following sequencing, reads were aligned to the genome and grouped by their molecular barcode. Fragments containing reads with the same molecular barcode that map to both the sense and anti-sense strand of the target were designed to have “duplex support.” Mutations were only scored if they were present in both strands (FIGS. 20 and 21).

### Example 2: Targeted DNA Sequencing of the Watson and Crick Strands of DNA

The identification and quantification of rare nucleic acid sequences is important for many areas of biology and clinical medicine. This example describes a method (termed SaferSeqS) that addresses this challenge by (i) efficiently introducing identical molecular barcodes in the Watson and Crick strands of template molecules and (ii) enriching for genomic regions of interest with a novel strand-specific PCR assay. It can be applied to evaluate mutations within a single amplicon or simultaneously within multiple amplicons, can assess limited quantities of DNA such as those found in plasma, and reduces the error rate of existing PCR-based molecular barcoding approaches by at least two orders of magnitude.

### Results

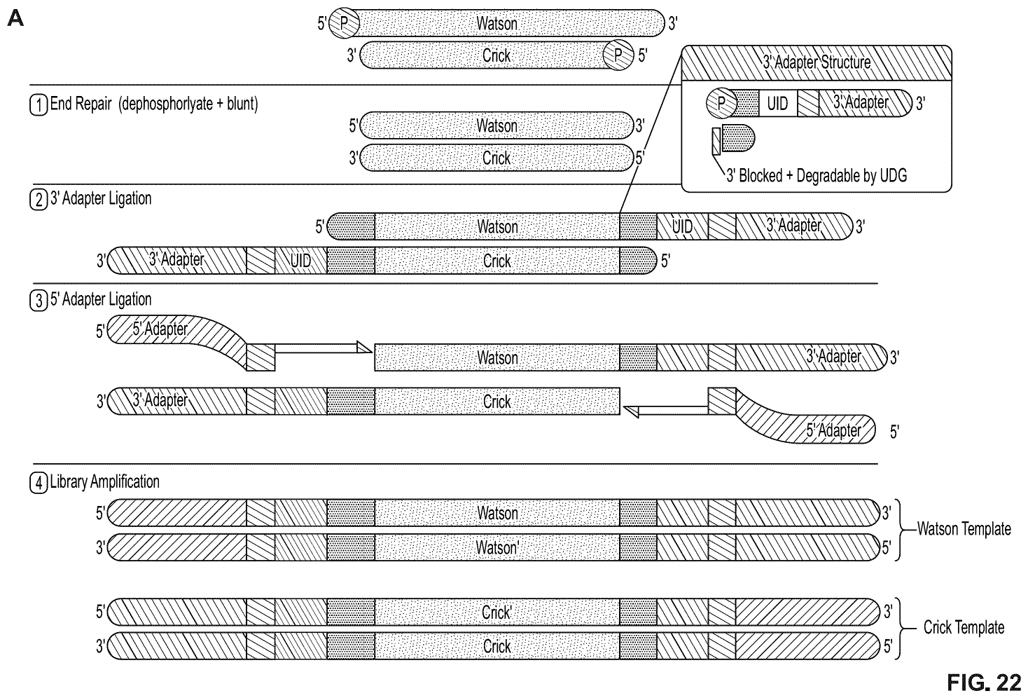

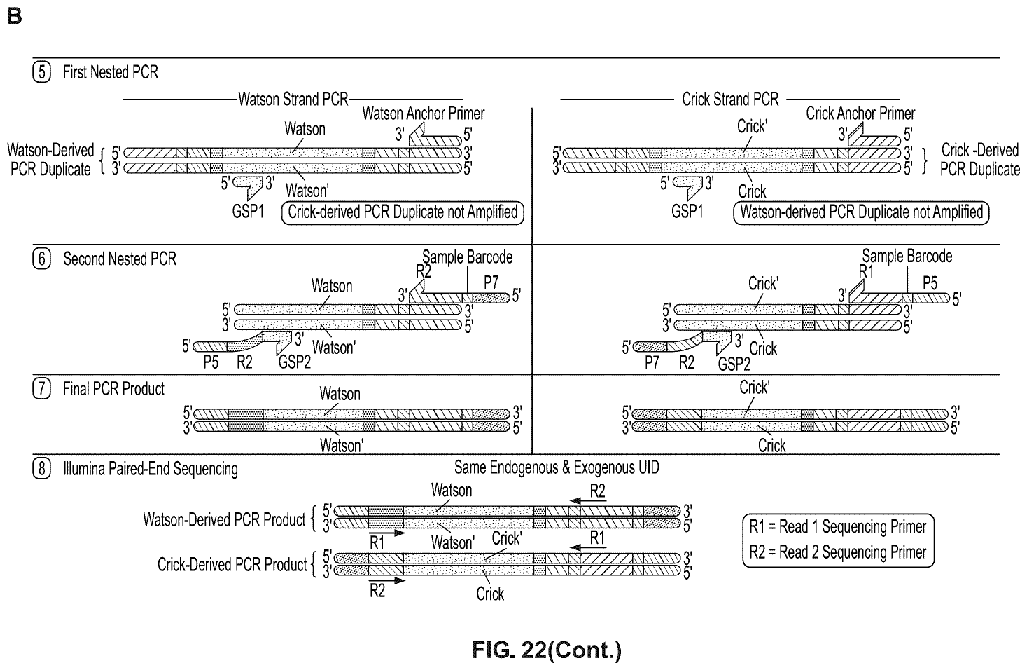

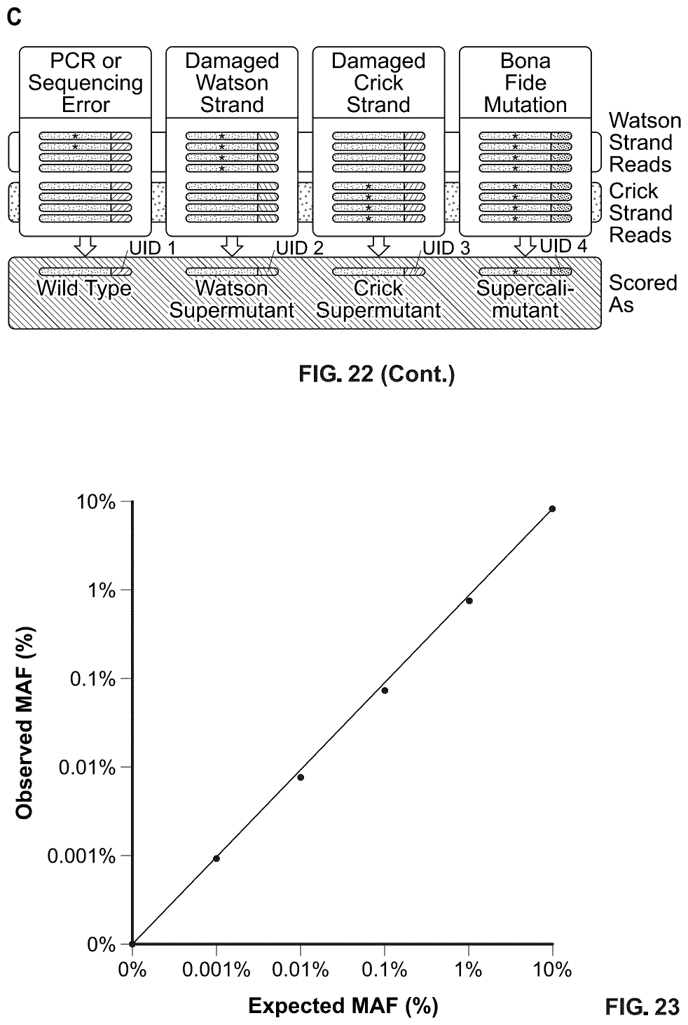

To address inefficiencies and introduced errors typically associated with library construction, a strategy was designed that involves the sequential ligation of adapter sequences to the 3′ and 5′ DNA fragment ends and the generation of double stranded molecular barcodes in situ (FIG. 22a). The in situ generation of molecular barcodes is a key innovation of the new library preparation method. The enzymes used for the in situ generation of double stranded molecular barcodes uniquely barcoded each DNA fragment and obviated the need to enzymatically prepare duplex adapters (FIG. 22a, steps 2 and 3). The adapter contained a stretch of 14 random nucleotides as the exogenous molecular barcode (unique identifier sequence [UID]). The adapter-ligated fragments were subjected to a limited of number of PCR cycles to create redundant copies of the two original DNA strands (FIG. 22a, step 4). For clarity, in this exemplary embodiment a UCSC reference sequence (available at genome.ucsc.edu/) was arbitrarily defined as the “Watson” strand and its reverse complement as the “Crick” strand.

Another innovation in this protocol is the use of a hemi-nested PCR-based approach for enrichment. Though hemi-nested PCR has previously been used for target enrichment (see, e.g., Zheng et al., 2014, Nat Med 20:1479-1484), major changes were required to apply it to duplex sequencing. In particular, two separate PCRs were performed—one for the Watson strand and one for the Crick strand. Both PCRs employed the same gene-specific primer, but each employed a different anchoring primer. PCR duplicates derived from each strand could be distinguished by the orientation of the insert relative to the exogenous UID (FIG. 22b).

Following sequencing, reads corresponding to each strand of the original DNA duplexes were grouped into Watson and Crick families. Each family member had the identical endogenous barcode representing the sequence at one end of the initial template fragment and the identical exogenous UID introduced in situ during library construction. Mutations present in >80% of a Watson strand family were called “Watson supermutants”. Mutations present in >80% of a Crick strand family were called “Crick supermutants”. Those present in >80% of both the Watson and Crick families with the same UID (a “duplex family”) were called “supercalifragilisticexpialidocious mutants”, referred to herein as “supercalimutants” (FIG. 22c).

As an initial demonstration of SaferSeqS, a mixing experiment was conducted in which DNA with a known mutation was spiked into DNA from a normal individual's leukocytes at ratios varying from 10% to 0%. These admixtures were predicted to result in 15,400, 150, 15, 15, 8 or 0 supercalimutants per assay. The fraction of on-target reads (i.e., reads comprised of the intended amplicon) was 88%, much higher than achievable with hybrid capture-based approaches (see, e.g., Samorodnitsky et al., 2015 Hum Mutat 36:903-914). Moreover, a strong correlation between the expected and observed allele frequencies was demonstrated across five orders of magnitude (FIG. 23, Pearson's r>0.999, p=2.02×10⁻¹²). Not a single mutant corresponding to the pre-specified admixed variant was observed in DNA from the normal individual, indicating very high specificity for the mutation of interest. Specificity was also determined for any base within the amplicon rather than just the queried base. Across a total of 37,747,670 bases queried among all DNA samples, only six supercalimutants were observed, representing a mutation frequency of 1.59×10⁻⁷ supercalimutants/bp (Table 7).

TABLE 7
Mutations identified in analytical sensitivity and specificity validation experiments.

| | | | Reference | Variant | | cDNA | Protein | # | Duplex | |
|---|---|---|---|---|---|---|---|---|---|---|
| Sample | Chrom | Position* | Base | Base | Gene | change | change | Supercalimutants | Depth | MAF |
| 10% Spike-in | chr17 | 7579507 | T | G | TP53 | c. 180A > C | p. P60P | 3,067 | 37,686 | 8.14E+00 |
| 1% Spike-in | chr17 | 7579507 | T | G | TP53 | c. 180A > C | p. P60P | 280 | 34,997 | 8.00E−01 |
| 1% Spike-in | chr17 | 7579560 | A | G | TP53 | c. 127T > C | p. L43L | 1 | 1,481 | 6.75E−02 |
| 0.1% Spike-in | chr17 | 7579507 | T | G | TP53 | c. 180A > C | p. P60P | 28 | 36,764 | 7.62E−02 |
| 0.01% Spike-in | chr17 | 7579507 | T | G | TP53 | c. 180A > C | p. P60P | 3 | 37,872 | 7.92E−03 |
| 0.001% Spike-in | chr17 | 7579507 | T | G | TP53 | c. 180A > C | p. P60P | 2 | 206,682 | 9.68E−04 |
| 0.001% Spike-in | chr17 | 7579518 | C | G | TP53 | c. 169G > C | p. D57H | 1 | 193,317 | 5.17E−04 |
| 0.001% Spike-in | chr17 | 7579547 | G | A | TP53 | c. 140C > T | p. P47L | 1 | 153,733 | 6.50E−04 |
| 0.001% Spike-in | chr17 | 7579575 | G | — | TP53 | c. 112del | p. Q38del | 1 | 37,951 | 2.63E−03 |
| 0.001% Spike-in | chr17 | 7579716 | G | A | TP53 | c. 80C > T | p. P27L | 1 | 14,207 | 7.04E−03 |
| 0% Spike-in | chr17 | 7579508 | G | C | TP53 | c. 179C > G | p. P60R | 1 | 200,754 | 4.98E−04 |

*Coordinates refer to the human reference genome hg19 release (Genome Reference Consortium GRCh37, February 2009).

It was then sought to determine whether SaferSeqS could be applied to clinical samples in which the quantity of DNA is limiting. For example, as little as 33 ng of DNA is often present in ten mL of cell-free plasma DNA samples used for liquid biopsies. The vast majority of DNA template molecules in these samples are wild type, with as few as one or two mutant templates among the 10,000 wild-type templates present in samples from patients with low tumor burdens. To sensitively detect this exceedingly small number of mutant templates, the assay should efficiently recover the starting molecules.

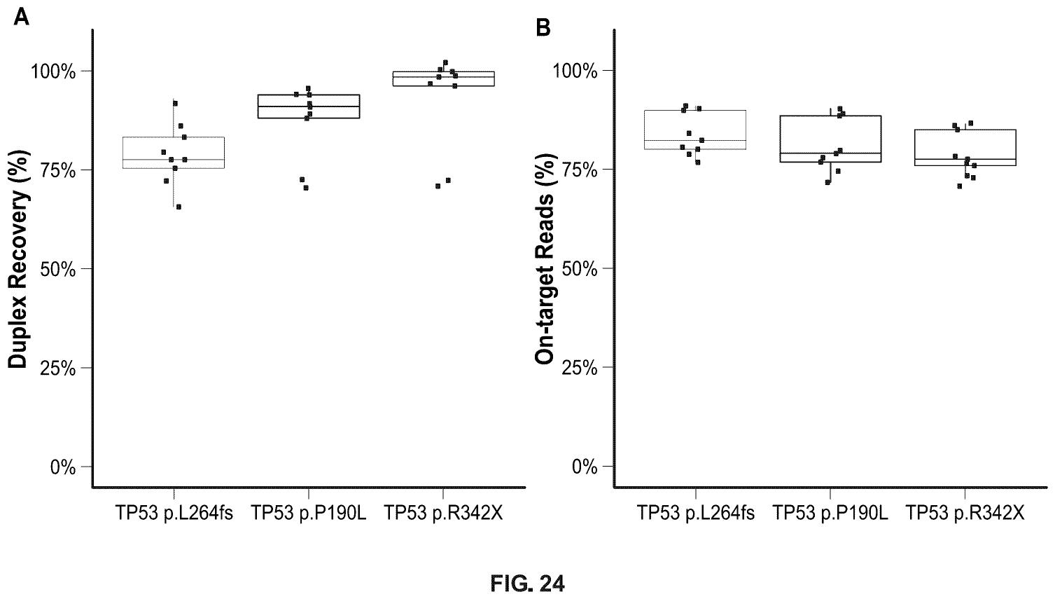

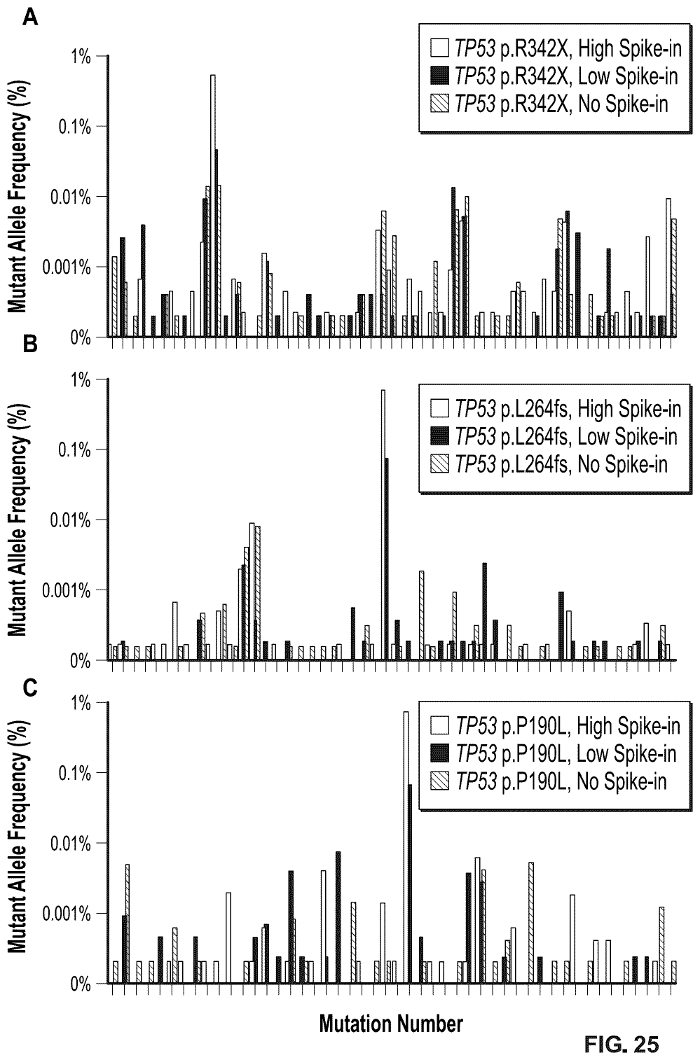

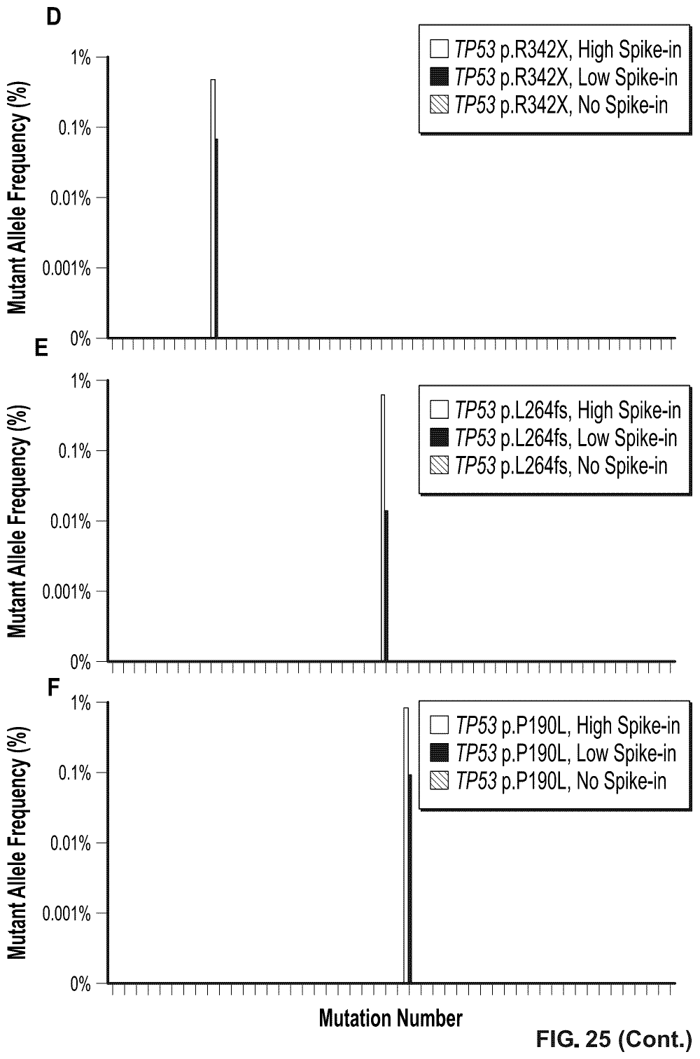

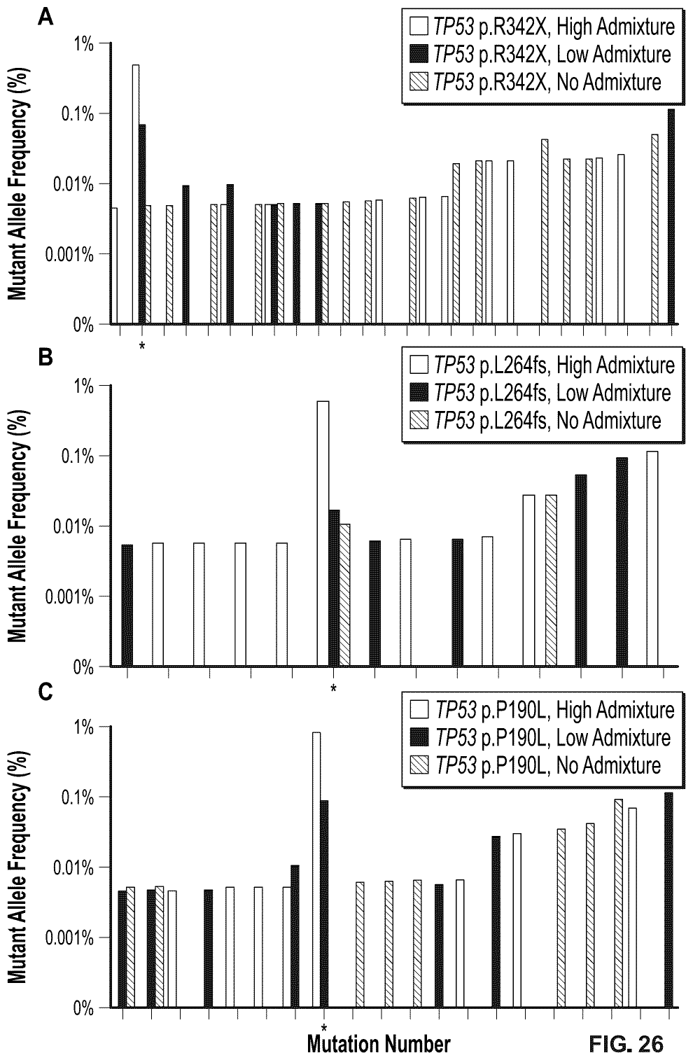

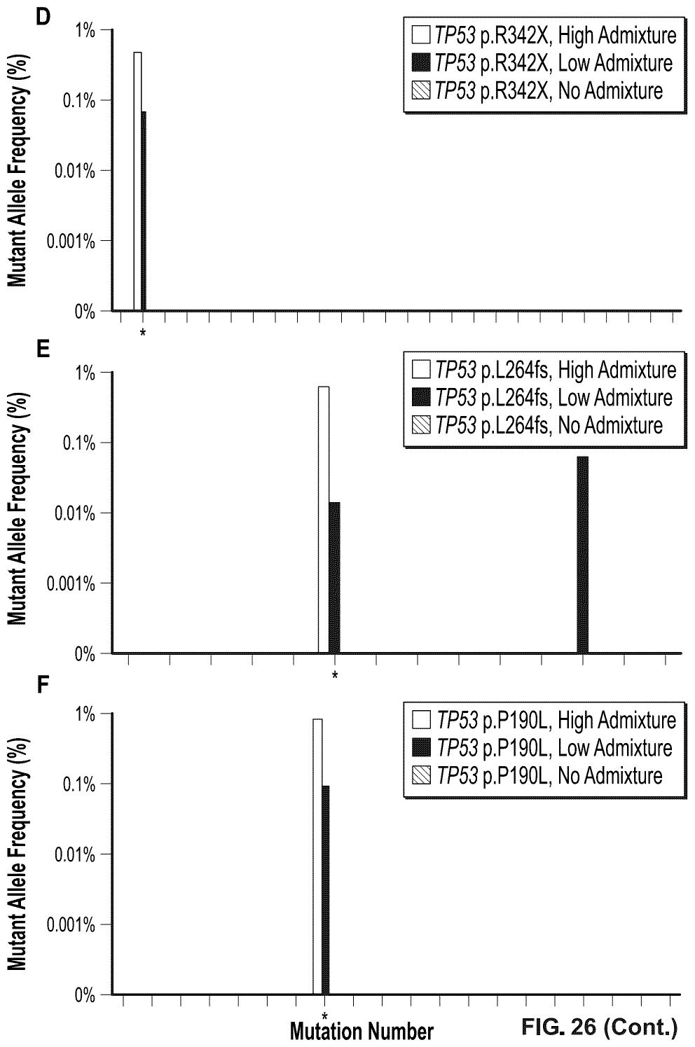

To assess SaferSeqS in such a challenging context, cell-free plasma DNA from cancer patients was mixed with cell-free plasma DNA from normal individuals to mimic mutation frequencies that are typically observed in clinical samples. In these experiments, 33 ng of each sample was assayed for one of three different mutations in TP53. The median fraction of on-target reads across the 27 experimental conditions (3 TP53 amplicons×3 samples×3 aliquots/sample) was 80% (range: 72% to 91%) (FIG. 24a). The median number of duplex families (i.e., both Watson and Crick strands containing the same endogenous and exogenous barcodes) was 89% (range: 65% to 102%) of the number of original template molecules (FIG. 24b). Moreover, in all six admixed samples, the supercalimutant of interest was identified at the expected frequency (FIG. 25b,d,e, Table 9). Mutations at this expected frequency were also identified in these same samples using a previously described, molecular barcoding method (“SafeSeqS” rather than “SaferSeqS”) (FIG. 25a,b,c, Table 8). An advantage of SaferSeqS was its specificity. There were a total of 1,406 supermutants representing 153 distinct mutations observed with the previously described method, reflecting an average error rate of 9.39×10⁻⁶ supermutants/bp (FIG. 25a,b,c, Table 8). The vast majority of these mutations were presumably polymerase errors that arose during early barcoding cycles in only one of the two strands. Similarly, if only Watson supermutants or Crick supermutants (i.e. those observed in only one of the two strands, FIG. 22c), rather than supercalimutants, were considered, an error rate of 6.56×10⁻⁶ supermutants/bp was observed (FIG. 26, Table 9). In contrast, only one supercalimutant was detected among 4,947,725 bases queried with SaferSeqS, representing an overall mutation rate of 2.02×10⁻⁷ (Table 9). These differences in specificity between SaferSeqS and previously described molecular barcoding methods (i.e. those employing direct PCR or adapter ligation to incorporate molecular barcodes prior to sequencing) were highly significant (P<3.5×10⁻¹⁰, two-sided Z test for proportions comparing SaferSeqS with each of the other methods).

Table 8. Comparison of mutations identified by SafeSeqS and SaferSeqS.

### (See Appendix A)

Table 9. Comparison of mutations identified by strand-agnostic molecular barcodes and SaferSeqS.

### (See Appendix B)

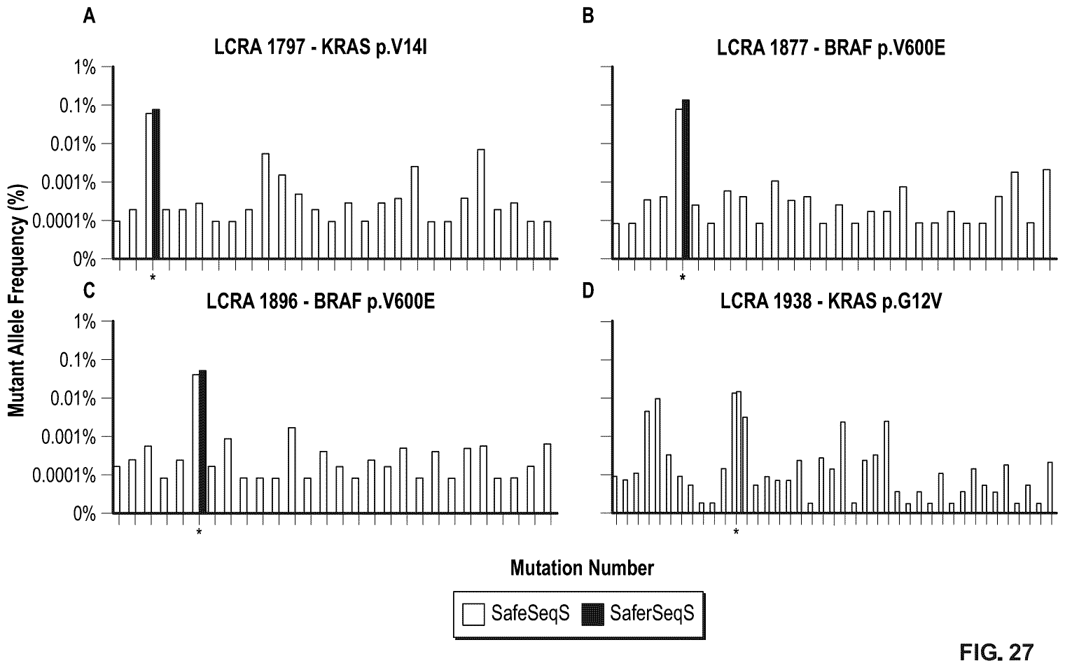

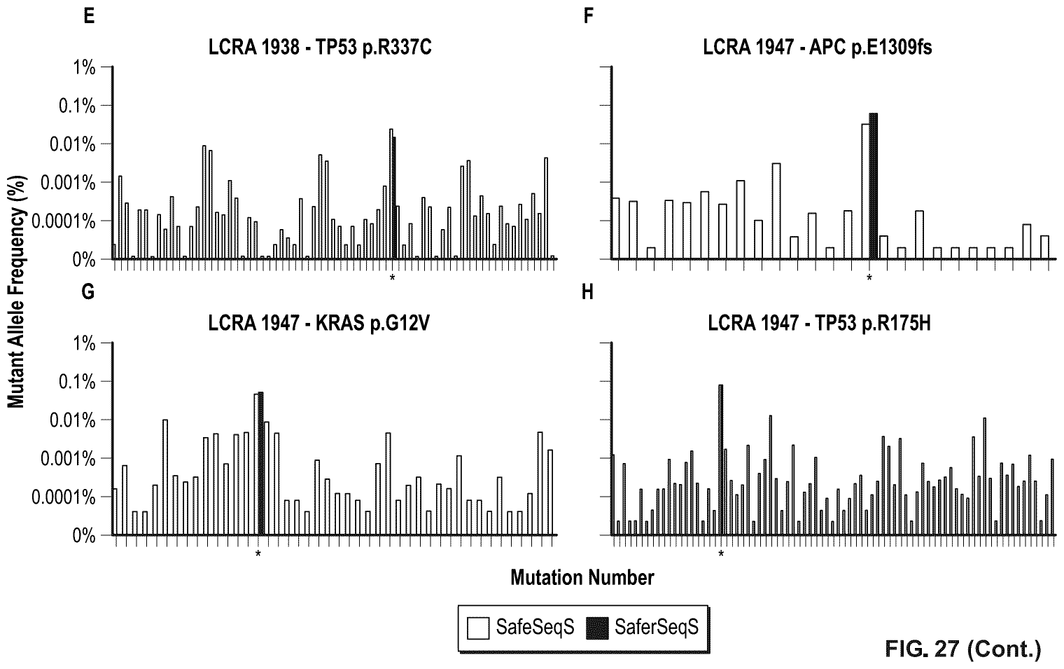

As a further demonstration of the clinical applicability of SaferSeqS, five cancer patients with minimal tumor burdens were evaluated. In each case, mutations in the primary tumors (rather than the plasma) were identified as described elsewhere (Tie et al., Sci Transl Med 8:346ra392 (2016)). The plasma from these patients was divided into two equal aliquots and evaluated one aliquot with a molecular barcoding method described elsewhere (Kinde et al., Proc Natl Acad Sci USA 108: 9530-9535 (2011)) and the other with SaferSeqS. In both cases, primers that resulted in small amplicons targeting the mutations of interest were designed. Evaluation with the previously described barcoding method revealed that the plasma samples harbored in aggregate eight mutations that were originally identified in the primary tumors. The frequencies of these mutations in the plasma varied from 0.01% to 0.1% (FIG. 27, Table 10). In addition to the eight known mutations, the previously described method identified 334 distinct mutations present at frequencies up to 0.013%, none of which were found in the primary tumors of these patients. These 334 mutations comprised 10,347 supermutants, reflecting an average error rate of 1.23×10⁻⁵ supermutants/bp (FIG. 27, Table 10). With SaferSeqS, the eight mutations found in the primary tumors were detected in all five patients at frequencies similar to those found with the previously described method (FIG. 27, Table 10). However, among the 8,707,755 queried bases, only one additional supercalimutant (rather than 334 mutations) was identified with SaferSeqS, representing an average error rate of 1.15×10⁻⁷ (Table 10). This >100-fold improvement in specificity over the previously described molecular barcoding method was highly significant (P<2.2×10⁻¹⁶, two-sided Z test for proportions).

Table 10. Mutations identified by SafeSeqS and SaferSeqS in plasma samples obtained from cancer patients.

### (See Appendix C)

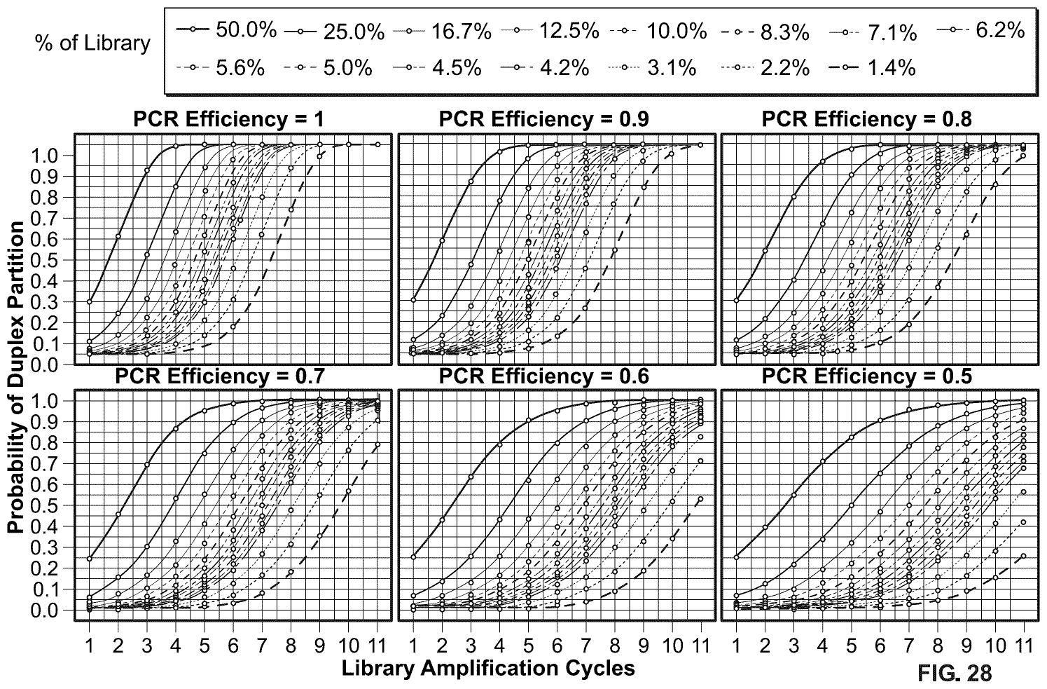

It was next examined whether SaferSeqS could simultaneously assay multiple targets, which can be useful for a variety of sequencing applications. SaferSeqS permits two types of multiplexing, one in which multiple targets are assayed in separate PCR reactions, and another in which multiple targets are assayed in the same PCR reaction. Because redundant Watson and Crick strand-derived copies are created during library amplification, the library can be partitioned into multiple PCR reactions without adversely impacting sample recovery. For example, assuming a PCR efficiency of 70%, up to 22 targets can be separately assayed with <10% loss in recovery if a DNA library is amplified with 11 PCR cycles (FIG. 28). In practice, either 100% or 4.4% of a library was assayed. The on-target rate was similar whether using 100% of 4.4% of the library, with 82% and 92% of reads properly mapping to the intended region. The number of duplex families recovered was also similar, with 7,825 and 6,769 recovered in the 100% and 4.4% library partitions.

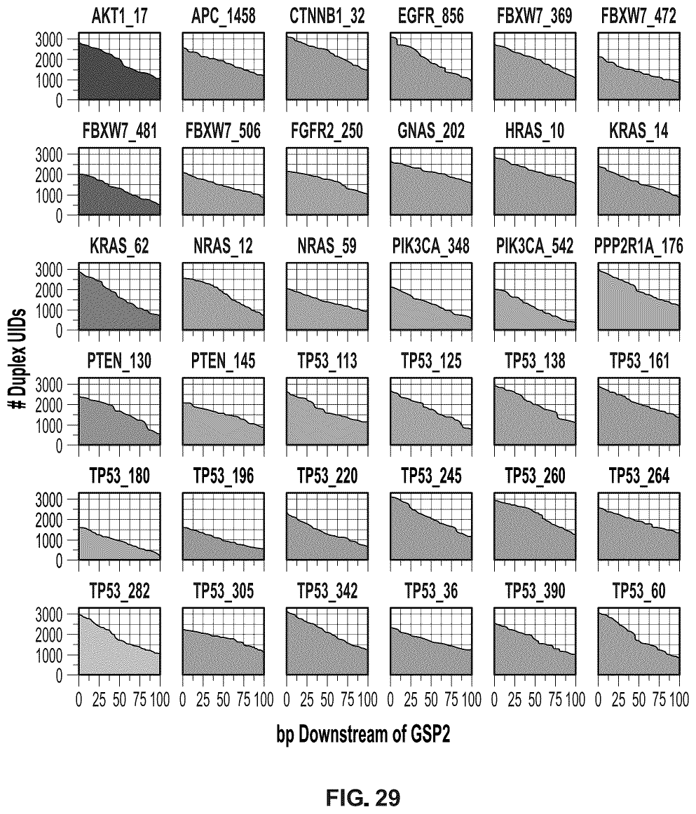

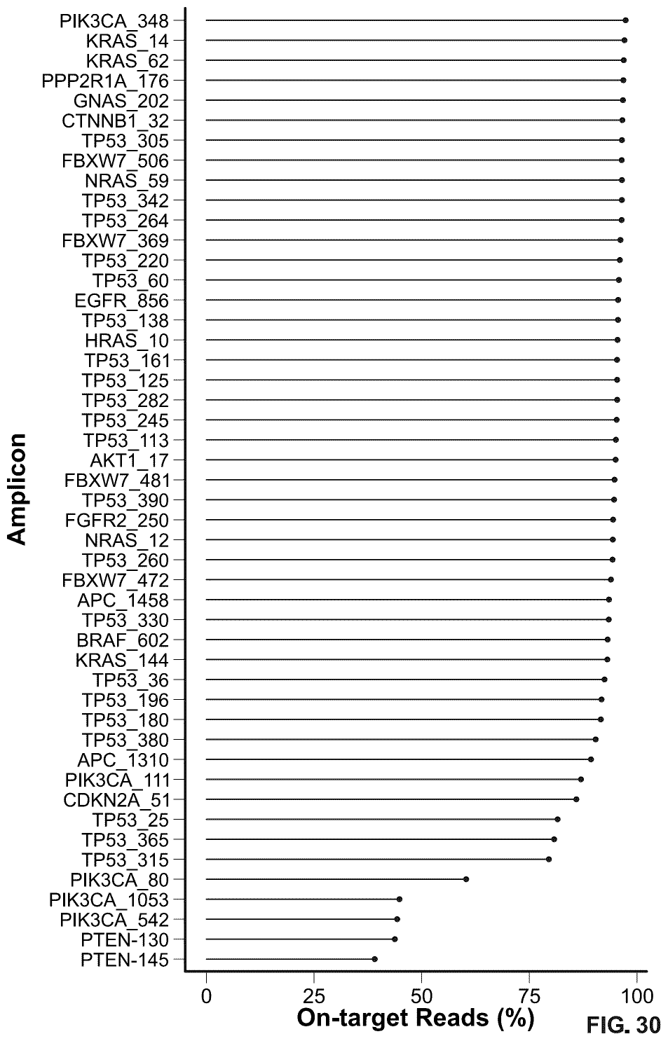

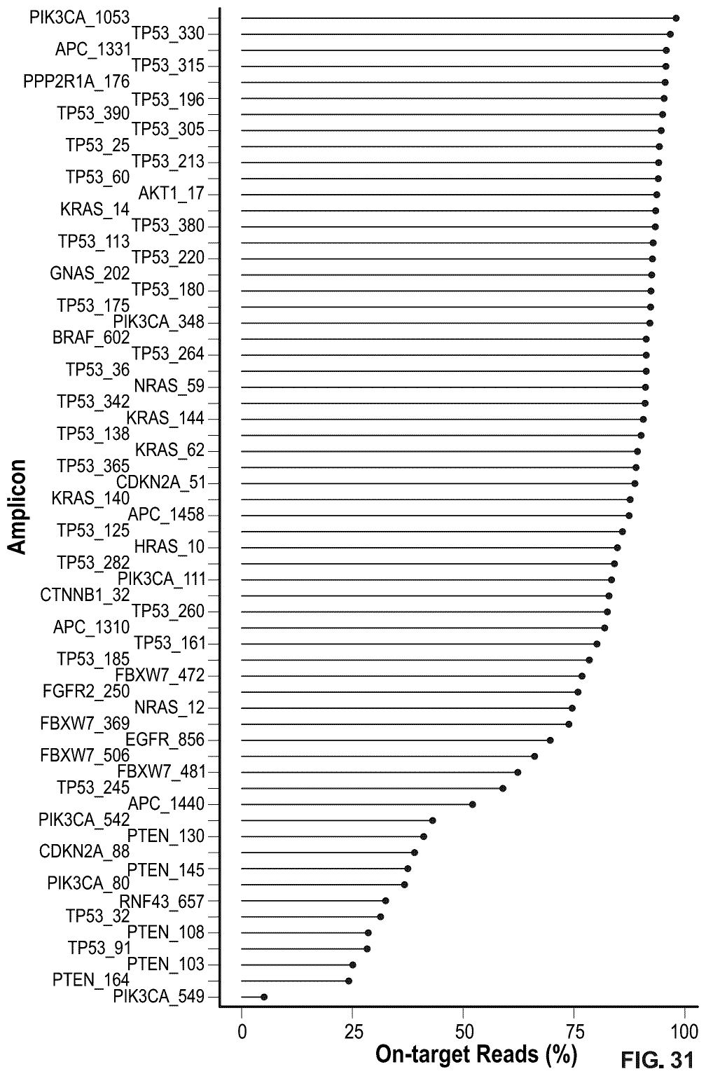

While the multiplexing approach described above is useful for simultaneously assaying a limited number of targets, applications which assess many genomic regions can include multiplexing into a small number of PCR reactions. To evaluate the multiplexing capabilities of SaferSeqS in this context, 48 primers were designed to query regions of driver genes that are commonly mutated in cancer (Table 11). These primers were combined in two reactions: one targeting 25 and the other targeting 23 regions. Each of the 48 primer pairs specifically amplified their intended targets (FIG. 30) and 36 were judged to be successful in that the number of duplex families was at least 50% of those identified with singleplex reactions. Of these 36, the median on-target rate for Watson-derived reads was 95% (range: 39% to 97%), and the median on-target rate for Crick-derived reads was 95% (range: 39% to 98%). Most importantly, the targets demonstrated relatively uniform recovery of the input molecules, with a coefficient of variation of only 17% (FIG. 29). The lengths of the amplicons sequenced (median of 77 bp, interquartile range: 71-83 bp) were also similar in all amplicons and consistent with the initial size of cell-free plasma DNA being ˜167 bp±10.4 bp (FIG. 29).

TABLE 11
Composition and GSP primer sequences of multiplex panel.

| | Multi- | | SEQ | | SEQ | | | GSP2 | GSP2 |
|---|---|---|---|---|---|---|---|---|---|
| | plex | GSP1 | ID | GSP2 | ID | GSP2 | GSP2 | Start | End |
| Amplicon | Set # | Sequence | NO: | Sequence | NO: | Chrom | Strand | Position* | Position* |
| AKT1_17 | 1 | TGCGTGGCTC | 6 | CACCCGCACG | 7 | chr14 | − | 105,246,554 | 105,246,570 |
| | | TCACC*A*C | | TCTGT*A*G | | | | | |
| APC_1310 | 1 | ACAGGATCTT | 8 | CTTCAGCTGA | 9 | chr5 | − | 112,175,223 | 112,175,243 |
| | | CAGCTGAC*C | | CCTAGTTCC* | | | | | |
| | | *T | | A*A | | | | | |
| CDKN2A_51 | 1 | CCTGGCTCTG | 10 | CCATTCTGTT | 11 | chr9 | − | 21,971,209 | 21,971,228 |
| | | ACCATTCTG* | | CTCTCTGG*C | | | | | |
| | | T*T | | *A | | | | | |
| EGFR_856 | 1 | AACACCGCAG | 12 | GCAGCATGTC | 13 | chr7 | + | 55,259,486 | 55,259,506 |
| | | CATGTC*A*A | | AAGATCACA* | | | | | |
| | | | | G*A | | | | | |
| FBXW7_369 | 1 | TTGTGCAGAG | 14 | GAGTTCAGTT | 15 | chr4 | + | 153,251,872 | 153,251,898 |
| | | TTCAGTTACC | | ACCTTAGGAG | | | | | |
| | | TT*A*G | | ATTTG*A*G | | | | | |
| FBXW7_481 | 1 | GGCCTGTCTC | 16 | GTCTCAATAT | 17 | chr4 | + | 153,247,333 | 153,247,356 |
| | | AATATCCCA* | | CCCAAACCCT | | | | | |
| | | A*A | | AA*G*A | | | | | |
| GNAS_202 | 1 | GGAACTTGGT | 18 | GGTCTCAAAG | 19 | chr20 | − | 57,484,426 | 57,484,449 |
| | | CTCAAAGATT | | ATTCCAGAAG | | | | | |
| | | C*C*A | | TC*A*G | | | | | |
| KRAS_14 | 1 | TCTGAATTAG | 20 | TATCGTCAAG | 21 | chr12 | + | 25,398,256 | 25,398,276 |
| | | CTGTATCGTC | | GCACTCTTG* | | | | | |
| | | AA*G*G | | C*C | | | | | |
| KRAS_144 | 1 | CTGTATTTAT | 22 | AGTGTTACTT | 23 | chr12 | + | 25,378,536 | 25,378,559 |
| | | TTCAGTGTTA | | ACCTGTCTTG | | | | | |
| | | CTTACCTGT* | | TC*T*T | | | | | |
| | | C*T | | | | | | | |
| NRAS_12 | 1 | GACTGAGTAC | 24 | AAACTGGTGG | 25 | chr1 | − | 115,258,751 | 115,258,769 |
| | | AAACTGGTG* | | TGGTTGG*A* | | | | | |
| | | G*T | | G | | | | | |
| PIK3CA_80 | 1 | TTTCGTAAGT | 26 | AGTGTTACTC | 27 | chr3 | + | 178,916,827 | 178,916,850 |
| | | GTTACTCAAG | | AAGAAGCAGA | | | | | |
| | | AA*G*C | | AA*G*G | | | | | |
| PIK3CA_348 | 1 | GCATCAGCAT | 28 | CATTTGACTT | 29 | chr3 | − | 178,921,563 | 178,921,590 |
| | | TTGACTTTAC | | TACCTTATCA | | | | | |
| | | CTTAT*C*A | | ATGTCT*C*G | | | | | |
| PIK3CA_1053 | 1 | GCATGCTGTT | 30 | TGTGTGGAAG | 31 | chr3 | − | 178,952,106 | 178,952,128 |
| | | TAATTGTGTG | | ATCCAATCCA | | | | | |
| | | *G*A | | T*T*T | | | | | |
| PTEN_145 | 1 | CCATAGAAAT | 32 | TAGGGCCTCT | 33 | chr10 | − | 89,692,953 | 89,692,972 |
| | | CTAGGGCCTC | | TGTGCCTT*T | | | | | |
| | | *T*T | | *A | | | | | |
| TP53_25 | 1 | GGCCTGCCCT | 34 | TTCCAATGGA | 35 | chr17 | + | 7,579,820 | 7,579,838 |
| | | TCCAA*T*G | | TCCACTC*A* | | | | | |
| | | | | C | | | | | |
| TP53_60 | 1 | GCAGCCTCTG | 36 | GGCATTCTGG | 37 | chr17 | + | 7,579,487 | 7,579,505 |
| | | GCATTC*T | | GAGCTTC*A* | | | | | |
| | | | | T | | | | | |
| TP53_125 | 1 | GCCAGGCATT | 38 | ATTGAAGTCT | 39 | chr17 | + | 7,579,272 | 7,579,292 |
| | | GAAGTCTC*A | | CATGGAAGC* | | | | | |
| | | *T | | C*A | | | | | |
| TP53_161 | 1 | CCGTCATGTG | 40 | GTGCTGTGAC | 41 | chr17 | + | 7,578,426 | 7,578,445 |
| | | CTGTGA*C*T | | TGCTTGTA*G | | | | | |
| | | | | *A | | | | | |
| TP53_196 | 1 | CCAAATACTC | 42 | CACACGCAAA | 43 | chr17 | + | 7,578,240 | 7,578,259 |
| | | CACACGCAA* | | TTTCCTTC*C | | | | | |
| | | A*T | | *A | | | | | |
| TP53_245 | 1 | GGAGTCTTCC | 44 | GTGATGATGG | 45 | chr17 | + | 7,577,514 | 7,577,533 |
| | | AGTGTGATGA | | TGAGGATG*G | | | | | |
| | | *T*G | | *G | | | | | |
| TP53_264 | 1 | CACAAACACG | 46 | ACGCACCTCA | 47 | chr17 | + | 7,577,119 | 7,577,137 |
| | | CACCTC*A*A | | AAGCTGT*T* | | | | | |
| | | | | C | | | | | |
| TP53_305 | 1 | AACTGCACCC | 48 | CCTCCACCGC | 49 | chr17 | + | 7,576,989 | 7,577,006 |
| | | TTGGTC*T*C | | TTCTTG*T*C | | | | | |
| TP53_330 | 1 | GAAACTTTCC | 50 | TGATAAGAGG | 51 | chr17 | + | 7,576,827 | 7,576,848 |
| | | ACTTGATAAG | | TCCCAAGACT | | | | | |
| | | AGG*T*C | | *T*A | | | | | |
| TP53_365 | 1 | AGTAGGGCCA | 52 | GGGCTGAGGT | 53 | chr17 | + | 7,573,910 | 7,573,926 |
| | | GGAAG*G*G | | CACTC*A*C | | | | | |
| TP53_390 | 1 | TGGGAGGCTG | 54 | GGGAACAAGA | 55 | chr17 | + | 7,572,906 | 7,572,926 |
| | | TCAGT*G*G | | AGTGGAGAA* | | | | | |
| | | | | T*G | | | | | |
| APC_1458 | 2 | TGCTTAGGTC | 56 | GTCCACTCTC | 57 | chr5 | − | 112,175,668 | 112,175,690 |
| | | CACTCTCT*C | | TCTCTTTTCA | | | | | |
| | | *T | | G*C*A | | | | | |
| BRAF_602 | 2 | ACAACTGTTC | 58 | ATGGGACCCA | 59 | chr7 | + | 140,453,111 | 140,453,128 |
| | | AAACTGATGG | | CTCCAT*C*G | | | | | |
| | | *G*A | | | | | | | |
| CTNNB1_32 | 2 | TTAGTCACTG | 60 | GGCAGCAACA | 61 | chr3 | + | 41,266,077 | 41,266,095 |
| | | GCAGCAAC*A | | GTCTTAC*C* | | | | | |
| | | *G | | T | | | | | |
| FBXW7_472 | 2 | TAGAGGAAGA | 62 | GAAGTCCCAA | 63 | chr4 | + | 153,249,328 | 153,249,349 |
| | | AGTCCCAAC* | | CCATGACAAG | | | | | |
| | | C*A | | *A*T | | | | | |
| FBXW7_506 | 2 | CTAACAACCC | 64 | CCCTCCTGCC | 65 | chr4 | + | 153,247,262 | 153,247,283 |
| | | TCCTGCCA*T | | ATCATATTGA | | | | | |
| | | *C | | *A*C | | | | | |
| FGFR2_250 | 2 | ACTCCTCCTT | 66 | TTTCTTCCCT | 67 | chr10 | − | 123,279,684 | 123,279,704 |
| | | TCTTCCCT*C | | CTCTCCACC* | | | | | |
| | | *T | | A*G | | | | | |
| HRAS_10 | 2 | GGAGCGATGA | 68 | GGAATATAAG | 69 | chr11 | − | 534,296 | 534,317 |
| | | CGGAATATAA | | CTGGTGGTGG | | | | | |
| | | *G*C | | *T*G | | | | | |
| KRAS_62 | 2 | CTCATGTACT | 70 | GTCCCTCATT | 71 | chr12 | + | 25,380,251 | 25,380,271 |
| | | GGTCCCTCAT | | GCACTGTAC* | | | | | |
| | | *T*G | | T*C | | | | | |
| NRAS_59 | 2 | GTGAAACCTG | 72 | CCTGTTTGTT | 73 | chr1 | − | 115,256,538 | 115,256,562 |
| | | TTTGTTGGAC | | GGACATACTG | | | | | |
| | | *A*T | | GAT*A*C | | | | | |
| PIK3CA_111 | 2 | CACGGTTGCC | 74 | GTTGCCTACT | 75 | chr3 | − | 178,916,911 | 178,916,934 |
| | | TACTGG*T*T | | GGTTCAATTA | | | | | |
| | | | | CT*T*T | | | | | |
| PIK3CA_542 | 2 | GAACAGCTCA | 76 | AATTTCTACA | 77 | chr3 | + | 178,936,057 | 178,936,080 |
| | | AAGCAATTTC | | CGAGATCCTC | | | | | |
| | | TAC*A*C | | TC*T*C | | | | | |
| PPP2RIA76_1 | 2 | CGACTCCCAG | 78 | TACTTCCGGA | 79 | chr19 | + | 52,715,940 | 52,715,959 |
| | | GTACTTC*C* | | ACCTGTGC*T | | | | | |
| | | G | | *C | | | | | |
| PTEN_130 | 2 | CATGTTGCAG | 80 | CAATTCACTG | 81 | chr10 | + | 89,692,878 | 89,692,901 |
| | | CAATTCACT* | | TAAAGCTGGA | | | | | |
| | | G*T | | AA*G*G | | | | | |
| TP53_36 | 2 | GGGACAGCAT | 82 | ATCAAATCAT | 83 | chr17 | + | 7,579,556 | 7,579,577 |
| | | CAAATCATC* | | CCATTGCTTG | | | | | |
| | | C*A | | *G*G | | | | | |
| TP53_113 | 2 | GTCACAGACT | 84 | TTGGCTGTCC | 85 | chr17 | + | 7,579,328 | 7,579,345 |
| | | TGGCTGT*C* | | CAGAAT*G*C | | | | | |
| | | C | | | | | | | |
| TP53_138 | 2 | ATCAACCCAC | 86 | TGCACAGGGC | 87 | chr17 | + | 7,578,499 | 7,578,515 |
| | | AGCTGC*A*C | | AGGTC*T*T | | | | | |
| TP53_180 | 2 | CCAGCTGCTC | 88 | TCACCATCGC | 89 | chr17 | + | 7,578,367 | 7,578,386 |
| | | ACCAT*C*G | | TATCTGAG*C | | | | | |
| | | | | *A | | | | | |
| TP53_220 | 2 | TGACAACCAC | 90 | CAGTTGCAAA | 91 | chr17 | + | 7,578,161 | 7,578,181 |
| | | CCTTAAC*C* | | CCAGACCTC* | | | | | |
| | | C | | A*G | | | | | |
| TP53_260 | 2 | GTGCAGGGTG | 92 | GGCAAGTGGC | 93 | chr17 | + | 7,577,482 | 7,577,498 |
| | | GCAAG*T*G | | TCCTG*A*C | | | | | |
| TP53_282 | 2 | CCTTTCTTGC | 94 | CGGAGATTCT | 95 | chr17 | + | 7,577,069 | 7,577,089 |
| | | GGAGATTCTC | | CTTCCTCTG* | | | | | |
| | | *T*T | | T*G | | | | | |
| TP53_315 | 2 | AAATATTCTC | 96 | CCAGTGGTTT | 97 | chr17 | + | 7,576,876 | 7,576,896 |
| | | CATCCAGTGG | | CTTCTTTGG* | | | | | |
| | | TT*T*C | | C*T | | | | | |
| TP53_342 | 2 | GGGCATCCTT | 98 | CTTGAGTTCC | 99 | chr17 | + | 7,573,974 | 7,573,993 |
| | | GAGTTCC*A* | | AAGGCCTC*A | | | | | |
| | | A | | *T | | | | | |
| TP53_380 | 2 | TCAGGCCCTT | 100 | CCTTCTGTCT | 101 | chr17 | + | 7,572,943 | 7,572,965 |
| | | CTGTCTTG*A | | TGAACATGAG | | | | | |
| | | *A | | T*T*T | | | | | |

*Coordinates refer to the human reference genome hg19 release (Genome Reference Consortium GRCh37, February 2009).
Asterisks within primer sequences designate phosphorothioate bonds.

Two exemplary methods can be used to assess multiple amplicons. The first involves parallel amplicon-specific PCR's in different wells. For liquid biopsies to monitor disease recurrence, when only a few driver gene mutations are typically observed, this strategy can be readily applied without concern about cross-hybridization among primers or other issues commonly encountered in multiplex PCR reactions. For other applications of liquid biopsies, such as screening when the mutation of interest is unknown, the evaluation of many more amplicons is useful; e.g., a combination of multiple primer pairs in each PCR well. This example has shown that at least 18 amplicons can be effectively analyzed in a single well with SaferSeqS, and hemi-nested PCR strategies not involving duplex sequencing have shown it is possible to co-amplify up to 313 amplicons.

By permitting efficient detection and quantification of rare genetic alterations, SaferSeqS can enable the development of highly sensitive and specific DNA-based molecular diagnostics as well as help answer a variety of important basic scientific questions.

### Methods

### Plasma and Peripheral Blood DNA Samples

DNA was purified from 10 mL plasma using cfPure MAX Cell-Free DNA Extraction Kit (BioChain, cat. #K5011625MA) as specified by the manufacturers. DNA from peripheral WBCs was purified with the QIAsymphony DSP DNA Midi Kit (Qiagen, cat. #937255) as specified by the manufacturer. Purified DNA from all samples was quantified as described elsewhere (see, e.g., Douville et al., 2019 bioRxiv, 660258).

### Library Preparation

A custom library preparation workflow was developed that could efficiently recover input DNA fragments and simultaneously incorporate double stranded molecular barcodes. In brief, duplex sequencing libraries were prepared with cell-free DNA or peripheral WBC DNA using an Accel-NGS 2S DNA Library Kit (Swift Biosciences, cat. #21024) with the following modifications: 1) DNA was pre-treated with three units of USER enzyme (New England BioLabs, cat. #M5505L) for 15 minutes at 37° C. to excise uracil bases; 2) the SPRI bead/PEG NaCl ratios used after each reaction were 2.0×, 1.8×, 1.2×, and 1.05× for End Repair 1, End Repair 2, Ligation 1, and Ligation 2, respectively; 3) a custom 50 μM 3′ adapter (Table 12) was substituted for Reagent Y2; 4) a custom 42 μM 5′ adapter (Table 12) was substituted for Reagent B2. The libraries were subsequently PCR-amplified in 50 μL reactions using primers targeting the ligated adapters (Table 12). The reaction conditions were as follows: 1×NEBNext Ultra II Q5 Master Mix (New England BioLabs, cat. #M0544L), 2 μM universal forward primer, and 2 μM universal reverse primer (Table 12). Libraries were amplified with 5, 7, or 11 cycles of PCR, depending on how many experiments were planned, according the following protocol: 98° C. for 30 seconds, cycles of 98° C. for 10 seconds, 65° C. for 75 seconds, and 4° C. hold. If 5 or 7 cycles were used, the libraries were amplified in single 50 μL reactions. If 11 cycles were used, the libraries were divided into eight aliquots and amplified in eight 50 μL reactions each supplemented with an additional 0.5 units of Q5@ Hot Start High-Fidelity DNA Polymerase (New England BioLabs, cat. #M0493L), 1 μL of 10 mM dNTPs (New England BioLabs, cat. #N0447L), and 0.4 μL of 25 mM MgCl₂ Solution (New England BioLabs, cat. #B9021S). The products were purified with 1.8×SPRI beads (Beckman Coulter cat. #B23317) and eluted into EB Buffer (Qiagen).

TABLE 12
Oligonucleotides for library construction,
strand-specific PCR assays, and sequencing.
Asterisks within primer sequences designate phosphorothioate bonds,
other custom modifications are indicated in the “Notes” column.

| | Oligo | SEQ | Purpose/ | |
|---|---|---|---|---|
| Oligo Name | Sequence | ID NO | Use | Notes |
| 3′ N14 | /5Phos/ATA | 102 | 1/2 of | /5Phos/ is a |
| Adapter | AAACGACGGC | | 3′ adapter | 5′ phosphate |
| Oligo #1 | NNNNNNNNNN | | containing | modification; |
| | NNNNAGATCG | | UID sequence | /3SpC3/ is a 3′ 3- |
| | GAAGAGCACA | | | carbon spacer |
| | CGTCTGAACT | | | modification |
| | CCAG*T*C/3 | | | |
| | SpC3/ | | | |
| 3′ Adapter | GCCGUCGUUU | 103 | 1/2 of | /33dT/ is a |
| Oligo #2 | UA/33dT/ | | 3′ adapter | 3′-deoxythymidine |
| | | | containing | modified base, |
| | | | 3′ block and | U represents |
| | | | degradable | deoxyuridine |
| | | | residues | |
| 5′ Adapter | ACACTCTTTC | 104 | 5′ adapter | |
| | CCTACACGAC | | ligation | |
| | GCTCTTCCGA | | | |
| | TCT | | | |
| Library | ACACTCTTTC | 105 | Library | |
| amplification | CCTACACGAC | | amplification | |
| primer #1 | GCTCTTCCGA | | | |
| | TCT | | | |
| Library | GACTGGAGTT | 106 | Library | |
| amplification | CAGACGTGTG | | amplification | |
| primer #2 | CTCTTCCGAT | | | |
| | CT | | | |
| GSP1 primer | See Table | 107 | First nested | Same primer used |
| | 11 | | gene-specific | in both Watson |
| | | | PCR primer | and Crick first |
| | | | | nested PCRs |
| Watson | GACTGGAGTT | 108 | Watson-specific | |
| GSP1 anchor | CAGACGT*G* | | anchor PCR | |
| primer | T | | primer | |
| Crick GSP1 | ACACTCTTTC | 109 | Crick-specific | |
| anchor | CCTACACG*A | | anchor PCR | |
| primer | *C | | primer | |
| Watson | AATGATACGG | 110 | Watson second | [GSP2] indicates |
| GSP2 primer | CGACCACCGA | | nested gene- | sequence of gene- |
| | GATCTACACX | | specific PCR | specific portion |
| | XXXXXXXCG | | primer | (see Table 11), Xs |
| | ACGTAAAACG | | | denote index sequence |
| | ACGGCCAGT | | | |
| | [GSP2] | | | |
| Crick GSP2 | CAAGCAGAAG | 111 | Crick second | [GSP2] indicates |
| primer | ACGGCATACG | | nested gene- | sequence of gene- |
| | AGATXXXXXX | | specific PCR | specific portion |
| | XXCACACAGG | | primer | (see Table 11), Xs |
| | AAACAGCTAT | | | denote index |
| | GACCATG | | | sequence |
| | [GSP2] | | | |
| Watson | CAAGCAGAAG | 112 | Watson second | Xs denote index |
| GSP2 | ACGGCATACG | | nested | sequence |
| anchor/ | AGATXXXXXX | | anchor and | |
| indexing | XXGTGACTGG | | indexing primer | |
| primer | AGTTCAGACG | | | |
| | TGTGCTCTTC | | | |
| | CGAT*C*T | | | |
| Crick GSP2 | AATGATACGG | 113 | Crick second | Xs denote index |
| anchor/ | CGACCACCGA | | nested anchor | sequence |
| indexing | GATCTACACX | | and indexing | |
| primer | XXXXXXXACA | | primer | |
| | CTCTTTCCCT | | | |
| | ACACGACGCT | | | |
| | CTTCCGAT*C | | | |
| | *T | | | |
| Custom Read | GAGATCTACA | 114 | Illumina | Spike into Illumina |
| 1 Sequencing | CNNNNNNNNC | | paired-end | standard read 1 |
| Primer | GACGTAAAAC | | sequencing | sequencing primer |
| | GACGGCCA*G | | | |
| | *T | | | |
| Custom | CATGGTCATA | 115 | Illumina | Spike into Illumina |
| Index | GCTGTTTCCT | | paired-end | standard index |
| Sequencing | GTG*T*G | | sequencing | sequencing primer |
| Primer | | | | |
| Custom Read | GGCATACGAG | 116 | Illumina | Spike into Illumina |
| 2 Sequencing | ATNNNNNNNN | | paired-end | standard read 2 |
| Primer | CACACAGGAA | | sequencing | sequencing primer |
| | ACAGCTATGA | | | |
| | CCA*T*G | | | |

### Library Construction

To address inefficiencies associated with library construction, a strategy that involves the sequential ligation of adapter sequences to the 3′ and 5′ DNA fragment ends and the generation of double stranded molecular barcodes in situ was designed (FIG. 22a). After DNA ends were dephosphorylated and repaired (FIG. 22a, step 1), an adapter was attached to the 3′ end of DNA fragments (FIG. 22a, step 2). The adapter was a partially double-stranded DNA fragment with end modifications that selectively ligated to the 3′ DNA ends and prevented adapter-dimer formation. Specifically, this adapter consisted of one oligonucleotide containing a 5′ phosphate end modification (Table 12, 3′ N14 Adapter Oligo #1) which was hybridized to another oligonucleotide containing a 3′ blocking group and deoxyuridines substituted for deoxythymidines (Table 12, 3′ N14 Adapter Oligo #2). This design permitted of the use of adapters at high concentration in the ligation reaction which promoted efficient attachment to the 3′ ends without the risk of significant dimer or concatemer formation. Furthermore, the adapter contained a stretch of 14 random nucleotides in one of the two oligonucleotides which compromised one strand of the duplex UID. Following ligation of the 3′ adapter, a second adapter (Table 12, 5′ Adapter) was ligated to the 5′ DNA fragment ends via a nick translation-like reaction consisting of a DNA polymerase, cohesive end-specific ligase, and uracil-DNA glycosylase (FIG. 22a, step 3). The concerted action of these enzymes synthesized the complementary strand of the UID, degraded the blocking portion of the 3′ adapter, and ligated the extended adapter to the 5′ DNA fragment end. The in situ generation of double stranded molecular barcodes uniquely barcoded each DNA fragment and obviated the need to enzymatically prepare duplex adapters, which has been noted to adversely affect input DNA recovery. Finally, the adapter-ligated fragments were subjected to a limited of number of PCR cycles to create redundant copies (UID “families”) of the two original DNA strands (FIG. 22a, step 4).

### Effects of Library Amplification Cycle Number and Efficiency

The number of PCR cycles and the efficiency of duplication during library amplification can be adjusted to optimize SaferSeqS parameters. Because SaferSeqS can involve partitioning of redundant Watson and Crick strand-derived copies into specific strand-specific PCRs for target enrichment, in preferred embodiments a requisite number of copies should be generated to ensure a high probability of duplex recovery. For example, assuming 100% efficiency, after one PCR cycle, each template DNA duplex is converted into two double stranded copies (one representing each strand), and there is only a 25% probability of properly distributing these two copies such that the one Watson strand-derived copy is partitioned into the Watson-specific PCR and the one Crick strand-derived copy is partitioned into the Crick-specific PCR. Increasing the number of PCR cycles, or increasing the amplification efficiency, generates more redundant copies which in turn increases the probability of recovering the original DNA duplex.

A probabilistic model was developed to estimate the number of PCR cycles and amplification efficiency necessary for efficient duplex recovery. This model consisted of three steps: 1) simulate the number of PCR progeny generated during library amplification; 2) randomly partition these PCR copies into Watson and Crick strand-specific reactions; and 3) determine the duplex recovery—that is, the proportion of original DNA duplexes which have at least one Watson strand-derived copy partitioned into the Watson strand-specific reaction(s) and at least one Crick strand-derived copy partitioned into the Crick strand-specific reaction(s).

The number of PCR copies of the original template strands generated during each library amplification cycle follows a binomial distribution. For the first PCR cycle, the number of strand-specific copies were initialized to one. It should be noted that the counts were initialized to one (instead of two) because the first library amplification cycle merely serves to denature the two original template strands and convert them into physically distinct double stranded forms. During the subsequent ith cycles of PCR, each of the nᵢ PCR copies can replicate with probability p (i.e. the efficiency of amplification) to generate a total of nᵢ₊₁ PCR copies equal to nᵢ+Binom(nᵢ, p). This process was iteratively repeated to simulate the number of progeny generated after i PCR cycles. Formally, the number of total PCR copies generated can be expressed as follows:

$${n}_{i} = {\sum }_{j = 1}^{i - 1} Binom ( {n}_{j} , p ) ; {n}_{1} = 1$$

After library amplification, each original DNA duplex has been amplified to generate n_{i,W} copies of the Watson strand and n_{i,C} copies of the Crick strand as described above. Each of the n_{i,W} and n_{i,C} copies are randomly partitioned into Watson and Crick strand-specific PCR reactions with a probability q that is equal to the fraction of the library used for each reaction. When the library is divided into a single Watson and single Crick strand-specific PCR, q equals 50%. If the library is divided into two Watson and Crick strand-specific PCRs, q equals 25%. The number of PCR copies that are partitioned into the appropriate strand-specific PCR (N_{k,W} or N_{k,C} for the kth Watson-specific or Crick-specific PCR, respectively) is drawn from a Binomial distribution with n_{i,W} or n_{i,C} “trials” and probability q of “success” for the Watson and Crick copies, respectively. Therefore, the probability of partitioning at least one Watson-derived PCR copy into the kth Watson-specific PCR reaction is:

$$P ( {N}_{k , W} > 0 ) = 1 - {( 1 - q )}^{{n}_{i , W}}$$

Similarly, the probability of partitioning at least one Crick-derived PCR copy into the kth Crick-specific PCR reaction is:

$$P ( {N}_{k , C} > 0 ) = 1 - {( 1 - q )}^{{n}_{i} , C}$$

Both strands of an original DNA duplex can only be recovered if N_{k,W} and N_{k,C} are greater than zero. Because the partitioning of the PCR progeny is independent, the probability duplex recovery is therefore predicted to be:

$$P ( {N}_{k , W} > 0 , {N}_{k , C} > 0 ) = [ 1 - {( 1 - q )}^{{n}_{i , W}} ] [ 1 - {( 1 - q )}^{{n}_{i , C}} ]$$

We varied the PCR efficiency from 100% to 50%, the number of library amplification cycles from 1 to 11, and the fraction of the library used for each reaction from 50% to 1.4%. For each condition, we conducted 10,000 simulations of the above described process and report the average duplex recovery in FIG. 28.

Fragment Size and Recovery with Anchored Hemi-Nested PCR

Anchored hemi-nested PCR theoretically demonstrates a higher recovery of template molecules than traditional amplicon PCR. In traditional amplicon PCR, a template molecule must contain the both forward and reverse primer binding sites and the intervening sequence that defines the amplicon. In contrast, in anchored hemi-nested PCR, the template molecules need only harbor the union of the two gene-specific primer binding sites in order to be recovered. The combined footprints of the nested gene-specific primers used in SaferSeqS are approximately 30 bp, whereas the amplicon lengths employed by SafeSeqS for profiling cfDNA are typically 70-80 bp. Formally, assuming uniformly random fragment start/end coordinates, the probability of recovering a template molecule of length L is

$$\frac{L - r}{L}$$

where r is the amplicon length in the case of traditional PCR or the length of the combined footprint of the gene specific primers in the case of anchored hemi-nested PCR. Thus, for cell-free DNA fragments of size ˜167 bp, anchored hemi-nested PCR can theoretically recover ˜25% more of the original template fragments than traditional amplicon PCR. Furthermore, unlike traditional amplicon PCR which produces predefined product sizes of that are dictated by the positions of the forward and reverse primers, anchored hemi-nested produces fragments of varying lengths with only one of the fragment ends dictated by the positions of the gene specific primers. Assuming template molecules of length L with uniformly random start/end coordinates, the observed fragment length after anchored hemi-nested PCR will be

$$\frac{L - r}{2}$$

where r is the length of the combined footprint of the gene specific primers.

### Exemplary Embodiment of SaferSeqS Bioinformatic Pipeline

In an exemplary embodiment of a SaferSeqS bioinformatics pipeline, the Watson and Crick reads for each sample were merged into a single BAM file and sorted by read name using SAMtools so that mate pairs could be readily extracted. Custom Python scripts were used for subsequent reconstruction of the duplex families and identification of Watson supermutants, Crick supermutants, and supercalimutants.

First, reads were grouped into UID families while taking note of which reads were derived from the Watson and Crick strand by examining the value of their bitwise flag (i.e. FLAG field). Reads containing bitwise flagwise values of 99 and 147 are derived from the Watson strand and those containing bitwise flags of 83 and 163 are derived from the Crick strand. Reads with any other bitwise flag values were excluded from subsequent analysis. Bitwise flags are numerical values that are assigned to read pairs during mapping. Their value indicates how the read mates align to the genome relative to one another. For example, if a read maps to the reference strand and its mate maps to the reverse (complementary) strand, then this read pair was derived from the Watson strand. Similarly, if a read maps to the reverse (complementary) strand and its mate maps to the reference strand, then this read pair was derived from the Crick strand.

Second, two additional quality control criteria were imposed during UID family grouping to improve determination of the endogenous molecular barcode (i.e. fragment end coordinate): 1) reads with soft clipping at the 5′ or 3′ of the fragment ends were excluded, 2) reads were required to contain the expected constant tag sequence (GCCGTCGTTTTAT; SEQ ID NO:117) immediately following the exogenous UID with no more than one mismatch.

Third, because in this example the number of possible exogenous UID sequences greatly exceeds the number of starting template molecules, “barcode collisions” in which two molecules share the same exogenous UID sequence but have different endogenous UIDs should be exceedingly rare. Specifically, the expected number of barcode collisions can be calculated from the classical “birthday problem” and is:

$$E [ X ] = n \{ 1 - {( 1 - \frac{1}{N} )}^{n - 1} \}$$

where n is equal to the number of template molecules and Nis equal to the number of possible barcodes. For a 14 bp exogenous UID sequence (comprising a total of 268,435,456 possible sequences) and 10,000 genome equivalents, the expected number of collisions is 0.37, or 0.0037% of the input. For this example, it was therefore required that each exogenous UID sequence could only be associated with one endogenous UID. In instances where an exogenous UID was associated with more than one endogenous UID, the largest family was preserved and all others were discarded.

It should be noted that in other experimental design parameters, non-unique exogenous UIDs may be used, and non-unique exogenous UID can be used in combination with endogenous UIDs for assignment into UID families.

Finally, because the exogenous barcodes themselves are susceptible to PCR and sequencing errors, we error-corrected UID sequences and regrouped the UID families using the UMI-tools network adjacency method.

After the reads were assembled into UID families, Watson supermutants, Crick supermutants, and supercalimutants were called as described elsewhere herein. To exclude common polymorphisms, all mutations in the Genome Aggregation Database (gnomeAD) present at an allele frequency greater than 0.1% were excluded. Reads comprising supercalimutants were subjected to a final manual inspection to exclude possible alignment artifacts.

### Estimates of Non-Clonal Somatic Mutation Rates

The DNA used in this study was obtained from a set of individuals of average age 30. As a result, the expected frequency of non-clonal somatic single base substitutions in these samples is 426 per diploid genome, or approximately 7×10⁻⁸ mutations per bp. In this study we evaluated a total of 42,695,395 bases with SaferSeqS from DNA derived from healthy control subjects. Among these 42,695,395 bases, 5 single base substitution supercalimutants, representing a mutation frequency of 12×10⁻⁸ were detected. To determine whether the frequency of supercalimutants observed is in accordance with previous estimates of non-clonal somatic mutation rates in healthy blood cells, the following exact one-sided binomial p-value was calculate:

$$P ( X \geq 5 ) = 1 - {\sum }_{k = 0}^{4} ( \begin{matrix}42 , 695 , 395 \\ k\end{matrix} ) {( 7 * 1 {0}^{- 8} )}^{k} {( 1 - 7 * 1 {0}^{- 8} )}^{42 , 695 , 395 - k} = 0.19$$

Therefore, there is no statistically significance difference between the number of supercalimutants observed and the predicted number of age-associated non-clonal somatic mutations arising from healthy hematopoietic stem cells.

### Anchored Hemi-Nested PCR

Target enrichment of the regions of interest was achieved using critical modifications of anchored hemi-nested PCR necessary for duplex sequencing. During the development of this custom stand-specific assay, various reaction conditions were optimized, including the number of cycles, the primer concentrations, and the polymerase formulation. The final optimized protocol was as follows: the first round of PCR was performed in a 50 μL reaction with following conditions: 1×NEBNext Ultra II Q5 Master Mix (New England BioLabs, cat. #M0544L), 2 μM GSP1 primer, and 2 μM P7 short anchor primer for amplification of the Watson strand. The GSP1 primer was specific for each amplicon, and the P7 short anchor primer was used as the anchor primer for the Watson strand of all amplicons (Tables 11 and 12). The Crick strand was amplified the same way, in a separate well, except that the P5 short primer anchor primer was substituted for the P7 short primer. Note that the GSP1 primer used for amplification of the Watson strand was identical to the GSP1 primer used for the Crick strand; the only difference between the Watson and Crick strand PCRs was the anchor primers. Both reactions (Watson and Crick strands) were amplified with 19 cycles according to the thermocycling protocol described above.

For the Watson strand, a second round of PCR was formed in 50 μl reactions using the identical reaction conditions used for the first round of PCR. The differences were (i) Template: 1% of product from the first anchored Watson strand PCR was used as the template (instead of the library used as template for the first PCR), and (ii) Primers: the gene-specific primers GSP2 were substituted for the GSP1 gene-specific primers and anchor P5 indexing primer was substituted for the P7 short anchor primer. The second round of PCR for the Crick strand was performed identically except that (i) Template: the first Crick strand PCR was used as the template and (ii) Primers: anchor P7 indexing primer was substituted for anchor P5 indexing primer. Both reactions (Watson and Crick strands) were amplified with 17 cycles according to the thermocycling protocol described previously. Sequences of the primers used for the second round of PCR are listed in Table 12. The products of the second round of PCR were pool and purified with 1.8×SPRI beads prior to sequencing.

For experiments in which multiple targets were simultaneously amplified within a single reaction, the PCR conditions were identical to those described above except: (i) each gene-specific primer was included at a final concentration of 0.25 μM, and (ii) the concentration of the anchor primer was included at a final concentration of 0.25 μM per target (e.g. at a final concentration of 6.25 μM if 25 targets were co-amplified).

### Sequencing

Library concentrations were determined using a KAPA Library Quantification Kit (KAPA Biosystems, cat. #KK4824) as described by the manufacturer. Sequencing was performed using 2×75 paired-end reads with eight base dual-indexing on an Illumina MiSeq instrument. A dual-indexed PhiX control library (SeqMatic cat. #TM-502-ND) was spiked in at 25% of the total templates to ensure base diversity across all cycles. Custom read 1, index, and read 2 sequencing primers (Table 12) were combined with standard Illumina sequencing primers at a final concentration of 1 μM.

### Mutation Calling and SaferSeqS Analysis Pipeline

Analysis of SafeSeqS data was performed as described elsewhere (see, e.g., Kinde et al., 2011 Proc Natl Acad Sci USA 108:9530-9535) using custom Python scripts. Sequencing reads underwent initial processing by extracting the first 14 nucleotides as the UID sequence and masking adapter sequences using Picard's IlluminaBasecallsToSam (broadinstitute.github.io/picard). Reads were then mapped to the hg19 reference genome using BWA-MEM (version 0.7.17) and sorted by UID sequence using SAMtools. UID families were scored if they consisted of 2 or more reads and if >90% of the reads mapped to the reference genome with the expected primer sequences. “Supermutants” were identified as mutations that were present in >95% of the mapped reads and had an average Phred score greater than 25.

A custom analysis pipeline was developed for the analysis of SaferSeqS. In brief, reads were demultiplexed, and the strand from which the reads were derived was identified using the index sequences. For clarity and succinctness, reads derived from the Watson strand are referred to as “Watson reads,” and reads derived from the Crick strand are referred to as “Crick reads.” For the Watson reads, the first 14 bases of read 1 were extracted as the UID sequence. Because the orientation of the insert is reversed for the Crick strand, the first 14 bases of read 2 were extracted as the UID sequence for the Crick reads. Adapter sequences were masked using Picard's IlluminaBasecallsToSam (broadinstitute.github.io/picard), and the resulting template-specific portions of the reads were mapped to the hg19 reference genome using BWA-MEM (version 0.7.17). Following alignment, the mapped Watson and Crick reads merged and sorted using SAMtools.

Python scripts were used for subsequent reconstruction of the duplex families and identification of Watson supermutants, Crick supermutants, and supercalimutants. After correcting for PCR and sequencing errors within the molecular barcode sequences as described elsewhere (see, e.g., Smith et al., 2017 Genome Res 27:491-499), Watson and Crick reads belonging to the same duplex family were grouped together to reconstruct the sequence of the original template molecule. To exclude artifacts stemming from the end repair step of library construction, bases fewer than 10 bases from the 3′ adapter sequence were not considered for mutation analysis. Watson and Crick supermutants were defined as mutations present in >80% of the Watson or Crick reads of a duplex family, respectively. Supercalimutants were defined as mutations present in >80% of both the Watson and Crick families with the same UID.

### Statistical Analyses

Continuous variables were reported as medians and range, while categorical variables were reported as whole numbers and percentages. All statistical tests were conducted using R's stats package (version 3.5.1).

These results demonstrate that SaferSeqS can detect rare mutations with extremely high specificity. The technique is highly scalable, cost effective, and amenable to high-throughput automation. SaferSeqS achieved up to a 5- to 75-fold improvement in input recovery over existing duplex sequencing techniques and can be applied to limited amounts of starting material, and resulted in a >50-fold improvement in error correction over standard PCR-based approaches employing molecular barcodes (FIG. 23, Table 8). It also offers a >50-fold improvement in error correction over optimal ligation-based techniques that employ only Watson or Crick supermutants rather than supercalimutants (FIG. 26, Table 9). Both reductions are useful for to the detection of mutations present at single or very low copy number, such as in cancer screening and minimal residual disease settings. Finally, because it incorporates duplex sequencing, SaferSeqS is considerably more sensitive than digital droplet PCR for the analysis of single amplicons, and, unlike digital droplet PCR, can be highly multiplexed.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### INCORPORATION BY REFERENCE

All references, issued patents and patent applications cited within the body of the instant specification are hereby incorporated by reference in their entirety, for all purposes.

What is claimed is:

1. A method, comprising:
a) amplifying a plurality of adapter-double-stranded cell-free DNA fragments (ADScfDFs) with first and second universal primers,
wherein each of said ADScfDFs comprise: a target Watson strand hybridized to a target Crick strand, a universal 3′ adapter attached to a 3′ end of said target Watson and Crick strands, and a universal 5′ adapter attached to a 5′ end of said target Watson and Crick strands, wherein said universal 3′ adapters each comprise a universal 3′ adapter sequence,
wherein attachment of said universal 3′ adapters followed by annealing and extending the universal 5′ adapters and ligating them to the 5′ ends to form the ADScfDFs provides a double stranded unique identifier (dsUID) sequence and single-stranded non-complementary 3′ and 5′ universal adapter sequences on each end of each of said ADScfDFs, wherein one strand of each dsUID is generated in situ after said universal 3′ adapters are attached to the strands and prior to said amplifying,
wherein said first universal primer is complementary to said universal 3′ adapter and said second universal primer is complementary to the complement of said universal 5′ adapter,
wherein said amplifying in step a) generates a duplex sequencing library sample comprising: i) a plurality of dsUID-labelled double-stranded templates (dsUID-dsTs);
b) amplifying a first aliquot from said duplex sequencing library sample with a first set of Watson target-selective primer pairs comprising: (i) a first Watson target-selective primer comprising a sequence complementary to the universal 3′ adapter, and (ii) a second Watson target-selective primer comprising a first Watson target-selective sequence, thereby: i) generating dsUID labelled double-stranded target Watson amplification products (dsUID-dsTWAPs) and ii) not generating dsUID labelled double-stranded target Crick amplification products (dsUID-dsTCAPs) in said first aliquot;
c) amplifying said dsUID-dsTWAPs with a second set of Watson target-selective primers comprising: (i) a third Watson target-selective primer comprising: A) a sequence complementary to said universal 3′ adapter, and B) a Watson sample index barcode, and (ii) a fourth Watson target-selective primer comprising a second Watson target-selective sequence that is nested with respect to said first Watson target-selective sequence, thereby creating a first sample comprising a plurality of dsUID-labelled double-stranded target Watson library members (dsUID-dsTWLMs);
d) amplifying a second aliquot from said duplex sequencing library sample with a first set of Crick target-selective primer pairs comprising: (i) a first Crick target-selective primer comprising a sequence complementary to the complement of said universal 5′ adapter, and (ii) a second Crick target-selective primer comprising a first Crick target-selective sequence that comprises the same target-selective sequence as said first Watson target-selective sequence, thereby: i) generating dsUID-dsTCAPs and ii) not generating said dsUID-dsTWAPs in said second aliquot;
e) amplifying said dsUID-dsTCAPs with a second set of Crick target-selective primers comprising: (i) a third Crick target-selective primer comprising: A) a sequence complementary to the complement of said universal 5′ adapter, and B) a Crick sample index barcode, and (ii) a fourth Crick target-selective primer comprising a second Crick target-selective sequence that is nested with respect to said first Crick target-selective sequence and comprises the same target-selective sequence as said second Watson target-selective sequence, thereby creating a second sample comprising a plurality of dsUID-double-stranded target Crick library members (dsUID-dsTCLMs);
f) pooling said first sample and said second sample to generate a combined sample comprising dsUID-dsTWLMs and dsUID-dsTCLMs;
g) sequencing said dsUID-dsTWLMs and dsUID-dsTCLMs to generate:
i) a plurality of UID-labeled Watson strand reads that comprise: A) a target Watson strand read, and B) a Watson sample index barcode read, and
ii) a plurality of UID-labeled Crick strand reads that comprise: A) a target Crick strand read, and B) a Crick sample index barcode read; and
h) processing said plurality of UID-labeled Watson strand reads and said UID-labeled Crick strand reads with a computer system to identify the presence of a mutation in one of said ADScfDFs, and identifying a mutation as present when present in said target Watson strand read and a corresponding mutation is present in said target Crick strand read.

2. The method of claim 1, wherein said attachment of said universal adapters comprises:
a) attaching 3′ adapters to 3′ ends of both Watson and Crick strands of a population of double-stranded cfDNA fragments in an analyte DNA sample,
wherein a first strand of the 3′ adapters comprises, in the 5′-3′ direction, (i) a first segment, (ii) a single stranded UID (ssUID) sequence, (iii) an annealing site for the universal 5′ adapter, and (iv) the universal 3′ adapter sequence, and
wherein the second strand of the 3′ adapters comprises, in the 5′ to 3′ direction, (i) a segment complementary to the first segment, and (ii) a 3′ blocking group,
b) annealing the universal 5′ adapters to said annealing site, wherein the universal 5′ adapters comprise, in the 5′ to 3′ direction, (i) a universal 5′ adapter sequence that is not complementary to the universal 3′ adapter sequence, and (ii) a sequence complementary to the annealing site for the universal 5′ adapter;
c) extending the universal 5′ adapters across said ssUID sequence and said first segment, thereby generating the complement of said ssUID sequence and complement of said first segment, and
d) covalently linking the 3′ end of said complement of said first segment to the 5′ ends of the Watson and Crick strands of the double-stranded cfDNA fragments having 3′ adapters attached to the 3′ ends, thereby attaching the universal 5′ adapter to said 5′ ends of said Watson and Crick strands.

3. The method of claim 1, wherein said amplifying in step a) employs no more than 50 cycles of PCR.

4. The method of claim 1, wherein said processing with said computer system comprises: i) assigning said UID-labeled Watson and Crick strand reads into UID families, wherein each member of a UID family comprises the same UID sequence.

5. The method of claim 4, wherein said processing with said computer system further comprises: ii) assigning said UID-labeled Watson and Crick strand reads of each UID family into a Watson subfamily and Crick subfamily based on the presence of said Watson sample index barcode read or said Crick sample index barcode read.

6. The method of claim 5, wherein said processing with said computer system further comprises: iii) identifying a first target Watson read as accurately representing a first target Watson strand of one of said ADScfDFs when at least 50% of members of the Watson subfamily contain the same nucleic acid sequence, and iv) identifying a first target Crick strand read as accurately representing a first target Crick strand of said one of said ADScfDFs when at least 50% of members of the Crick subfamily contain the same nucleic acid sequence.

7. The method of claim 6, wherein said processing with said computer system further comprises: v) performing at least one of the following:
A) identifying the presence of a first Watson variant in said first target Watson strand read when said first target Watson strand read differs from a reference sequence that lacks said first Watson variant, and/or
B) identifying the presence of a first Crick variant in said first target Crick strand read when said first target Crick read differs from a reference sequence that lacks said first Crick variant.

8. The method of claim 7, wherein said processing with said computer system further comprises identifying the presence of a first bonafide mutation in one of said ADScfDFs when both said first Watson variant and said first Crick variant are present.

9. The method of claim 6, wherein said at least 50% is greater than 80%.

10. The method of claim 1, wherein said third Watson target-selective primer further comprises: C) a first grafting sequence that permits hybridization to a first grafting primer on a sequencer.

11. The method of claim 10, wherein said third Crick target-selective primer further comprises: C) a second grafting sequence that permits hybridization to a second grafting primer on said sequencer.

12. The method of claim 1, wherein cell free DNA fragments are from a plasma sample.

13. The method of claim 1, wherein the identified mutation is present in a population of double-stranded DNA fragments at a frequency of 0.1% or less.

14. The method of claim 1, wherein the identified mutation is present in a population of double-stranded DNA fragments at a frequency of 0.1% to 0.01%.

15. The method of claim 1, wherein the identified mutation is present in a population of double-stranded DNA fragments at a frequency of 0.1% to 0.00001%.

16. The method of claim 1, wherein no hybridization capture is employed at any point during the method.

17. The method of claim 1, wherein said processing with said computer system further comprises reporting the presence of said first bonafide mutation.

18. The method of claim 1, wherein said amplifying in step a) employs no more than 18 cycles of PCR.

19. The method of claim 1, wherein said amplifying in step a) employs no more than 11 cycles of PCR.

20. The method of claim 1, wherein said amplifying in step a) employs no more than 7 cycles of PCR.


SEQUENCE LISTING (117 sequences)

SEQ ID NO: 1 - DNA, 59 bp, synthetic construct
  misc_difference 57..58: note = Residues 57-58 are connected by a phosphorothioate bond
  misc_difference 58..59: note = Residues 58-59 are connected by a phosphorothioate bond
  source 1..59: mol_type = other DNA
ataaaacgacggcnnnnnnnnnnnnnnagatcggaagagcacacgtctgaactccagtc

SEQ ID NO: 2 - skipped

SEQ ID NO: 3 - DNA, 13 bp, synthetic construct
  modified_base 5: mod_base = OTHER; note = deoxyuridine
  modified_base 8: mod_base = OTHER; note = deoxyuridine
  modified_base 9: mod_base = OTHER; note = deoxyuridine
  modified_base 10: mod_base = OTHER; note = deoxyuridine
  modified_base 11: mod_base = OTHER; note = deoxyuridine
  modified_base 13: mod_base = OTHER; note = 3'-deoxythymidine
  source 1..13: mol_type = other DNA
gccgtcgttttat

SEQ ID NO: 4 - DNA, 33 bp, synthetic construct
  source 1..33: mol_type = other DNA
acactctttccctacacgacgctcttccgatct

SEQ ID NO: 5 - DNA, 32 bp, synthetic construct
  source 1..32: mol_type = other DNA
gactggagttcagacgtgtgctcttccgatct

SEQ ID NO: 6 - DNA, 17 bp, synthetic construct
  source 1..17: mol_type = other DNA
tgcgtggctctcaccac

SEQ ID NO: 7 - DNA, 17 bp, synthetic construct
  source 1..17: mol_type = other DNA
cacccgcacgtctgtag

SEQ ID NO: 8 - DNA, 20 bp, synthetic construct
  source 1..20: mol_type = other DNA
acaggatcttcagctgacct

SEQ ID NO: 9 - DNA, 21 bp, synthetic construct
  source 1..21: mol_type = other DNA
cttcagctgacctagttccaa

SEQ ID NO: 10 - DNA, 21 bp, synthetic construct
  source 1..21: mol_type = other DNA
cctggctctgaccattctgtt

SEQ ID NO: 11 - DNA, 20 bp, synthetic construct
  source 1..20: mol_type = other DNA
ccattctgttctctctggca

SEQ ID NO: 12 - DNA, 18 bp, synthetic construct
  source 1..18: mol_type = other DNA
aacaccgcagcatgtcaa

SEQ ID NO: 13 - DNA, 21 bp, synthetic construct
  source 1..21: mol_type = other DNA
gcagcatgtcaagatcacaga

SEQ ID NO: 14 - DNA, 24 bp, synthetic construct
  source 1..24: mol_type = other DNA
ttgtgcagagttcagttaccttag

SEQ ID NO: 15 - DNA, 27 bp, synthetic construct
  source 1..27: mol_type = other DNA
gagttcagttaccttaggagatttgag

SEQ ID NO: 16 - DNA, 21 bp, synthetic construct
  source 1..21: mol_type = other DNA
ggcctgtctcaatatcccaaa

SEQ ID NO: 17 - DNA, 24 bp, synthetic construct
  source 1..24: mol_type = other DNA
gtctcaatatcccaaaccctaaga

SEQ ID NO: 18 - DNA, 23 bp, synthetic construct
  source 1..23: mol_type = other DNA
ggaacttggtctcaaagattcca

SEQ ID NO: 19 - DNA, 24 bp, synthetic construct
  source 1..24: mol_type = other DNA
ggtctcaaagattccagaagtcag

SEQ ID NO: 20 - DNA, 24 bp, synthetic construct
  source 1..24: mol_type = other DNA
tctgaattagctgtatcgtcaagg

SEQ ID NO: 21 - DNA, 21 bp, synthetic construct
  source 1..21: mol_type = other DNA
tatcgtcaaggcactcttgcc

SEQ ID NO: 22 - DNA, 31 bp, synthetic construct
  source 1..31: mol_type = other DNA
ctgtatttatttcagtgttacttacctgtct

SEQ ID NO: 23 - DNA, 24 bp, synthetic construct
  source 1..24: mol_type = other DNA
agtgttacttacctgtcttgtctt

SEQ ID NO: 24 - DNA, 21 bp, synthetic construct
  source 1..21: mol_type = other DNA
gactgagtacaaactggtggt

SEQ ID NO: 25 - DNA, 19 bp, synthetic construct
  source 1..19: mol_type = other DNA
aaactggtggtggttggag

SEQ ID NO: 26 - DNA, 24 bp, synthetic construct
  source 1..24: mol_type = other DNA
tttcgtaagtgttactcaagaagc

SEQ ID NO: 27 - DNA, 24 bp, synthetic construct
  source 1..24: mol_type = other DNA
agtgttactcaagaagcagaaagg

SEQ ID NO: 28 - DNA, 27 bp, synthetic construct
  source 1..27: mol_type = other DNA
gcatcagcatttgactttaccttatca

SEQ ID NO: 29 - DNA, 28 bp, synthetic construct
  source 1..28: mol_type = other DNA
catttgactttaccttatcaatgtctcg

SEQ ID NO: 30 - DNA, 22 bp, synthetic construct
  source 1..22: mol_type = other DNA
gcatgctgtttaattgtgtgga

SEQ ID NO: 31 - DNA, 23 bp, synthetic construct
  source 1..23: mol_type = other DNA
tgtgtggaagatccaatccattt

SEQ ID NO: 32 - DNA, 22 bp, synthetic construct
  source 1..22: mol_type = other DNA
ccatagaaatctagggcctctt

SEQ ID NO: 33 - DNA, 20 bp, synthetic construct
  source 1..20: mol_type = other DNA
tagggcctcttgtgccttta

SEQ ID NO: 34 - DNA, 17 bp, synthetic construct
  source 1..17: mol_type = other DNA
ggcctgcccttccaatg

SEQ ID NO: 35 - DNA, 19 bp, synthetic construct
  source 1..19: mol_type = other DNA
ttccaatggatccactcac

SEQ ID NO: 36 - DNA, 17 bp, synthetic construct
  source 1..17: mol_type = other DNA
gcagcctctggcattct

SEQ ID NO: 37 - DNA, 19 bp, synthetic construct
  source 1..19: mol_type = other DNA
ggcattctgggagcttcat

SEQ ID NO: 38 - DNA, 20 bp, synthetic construct
  source 1..20: mol_type = other DNA
gccaggcattgaagtctcat

SEQ ID NO: 39 - DNA, 21 bp, synthetic construct
  source 1..21: mol_type = other DNA
attgaagtctcatggaagcca

SEQ ID NO: 40 - DNA, 18 bp, synthetic construct
  source 1..18: mol_type = other DNA
ccgtcatgtgctgtgact

SEQ ID NO: 41 - DNA, 20 bp, synthetic construct
  source 1..20: mol_type = other DNA
gtgctgtgactgcttgtaga

SEQ ID NO: 42 - DNA, 21 bp, synthetic construct
  source 1..21: mol_type = other DNA
ccaaatactccacacgcaaat

SEQ ID NO: 43 - DNA, 20 bp, synthetic construct
  source 1..20: mol_type = other DNA
cacacgcaaatttccttcca

SEQ ID NO: 44 - DNA, 22 bp, synthetic construct
  source 1..22: mol_type = other DNA
ggagtcttccagtgtgatgatg

SEQ ID NO: 45 - DNA, 20 bp, synthetic construct
  source 1..20: mol_type = other DNA
gtgatgatggtgaggatggg

SEQ ID NO: 46 - DNA, 18 bp, synthetic construct
  source 1..18: mol_type = other DNA
cacaaacacgcacctcaa

SEQ ID NO: 47 - DNA, 19 bp, synthetic construct
  source 1..19: mol_type = other DNA
acgcacctcaaagctgttc

SEQ ID NO: 48 - DNA, 18 bp, synthetic construct
  source 1..18: mol_type = other DNA
aactgcacccttggtctc

SEQ ID NO: 49 - DNA, 18 bp, synthetic construct
  source 1..18: mol_type = other DNA
cctccaccgcttcttgtc

SEQ ID NO: 50 - DNA, 25 bp, synthetic construct
  source 1..25: mol_type = other DNA
gaaactttccacttgataagaggtc

SEQ ID NO: 51 - DNA, 22 bp, synthetic construct
  source 1..22: mol_type = other DNA
tgataagaggtcccaagactta

SEQ ID NO: 52 - DNA, 17 bp, synthetic construct
  source 1..17: mol_type = other DNA
agtagggccaggaaggg

SEQ ID NO: 53 - DNA, 17 bp, synthetic construct
  source 1..17: mol_type = other DNA
gggctgaggtcactcac

SEQ ID NO: 54 - DNA, 17 bp, synthetic construct
  source 1..17: mol_type = other DNA
tgggaggctgtcagtgg

SEQ ID NO: 55 - DNA, 21 bp, synthetic construct
  source 1..21: mol_type = other DNA
gggaacaagaagtggagaatg

SEQ ID NO: 56 - DNA, 20 bp, synthetic construct
  source 1..20: mol_type = other DNA
tgcttaggtccactctctct

SEQ ID NO: 57 - DNA, 23 bp, synthetic construct
  source 1..23: mol_type = other DNA
gtccactctctctcttttcagca

SEQ ID NO: 58 - DNA, 22 bp, synthetic construct
  source 1..22: mol_type = other DNA
acaactgttcaaactgatggga

SEQ ID NO: 59 - DNA, 18 bp, synthetic construct
  source 1..18: mol_type = other DNA
atgggacccactccatcg

SEQ ID NO: 60 - DNA, 20 bp, synthetic construct
  source 1..20: mol_type = other DNA
ttagtcactggcagcaacag

SEQ ID NO: 61 - DNA, 19 bp, synthetic construct
  source 1..19: mol_type = other DNA
ggcagcaacagtcttacct

SEQ ID NO: 62 - DNA, 21 bp, synthetic construct
  source 1..21: mol_type = other DNA
tagaggaagaagtcccaacca

SEQ ID NO: 63 - DNA, 22 bp, synthetic construct
  source 1..22: mol_type = other DNA
gaagtcccaaccatgacaagat

SEQ ID NO: 64 - DNA, 20 bp, synthetic construct
  source 1..20: mol_type = other DNA
ctaacaaccctcctgccatc

SEQ ID NO: 65 - DNA, 22 bp, synthetic construct
  source 1..22: mol_type = other DNA
ccctcctgccatcatattgaac

SEQ ID NO: 66 - DNA, 20 bp, synthetic construct
  source 1..20: mol_type = other DNA
actcctcctttcttccctct

SEQ ID NO: 67 - DNA, 21 bp, synthetic construct
  source 1..21: mol_type = other DNA
tttcttccctctctccaccag

SEQ ID NO: 68 - DNA, 22 bp, synthetic construct
  source 1..22: mol_type = other DNA
ggagcgatgacggaatataagc

SEQ ID NO: 69 - DNA, 22 bp, synthetic construct
  source 1..22: mol_type = other DNA
ggaatataagctggtggtggtg

SEQ ID NO: 70 - DNA, 22 bp, synthetic construct
  source 1..22: mol_type = other DNA
ctcatgtactggtccctcattg

SEQ ID NO: 71 - DNA, 21 bp, synthetic construct
  source 1..21: mol_type = other DNA
gtccctcattgcactgtactc

SEQ ID NO: 72 - DNA, 22 bp, synthetic construct
  source 1..22: mol_type = other DNA
gtgaaacctgtttgttggacat

SEQ ID NO: 73 - DNA, 25 bp, synthetic construct
  source 1..25: mol_type = other DNA
cctgtttgttggacatactggatac

SEQ ID NO: 74 - DNA, 18 bp, synthetic construct
  source 1..18: mol_type = other DNA
cacggttgcctactggtt

SEQ ID NO: 75 - DNA, 24 bp, synthetic construct
  source 1..24: mol_type = other DNA
gttgcctactggttcaattacttt

SEQ ID NO: 76 - DNA, 25 bp, synthetic construct
  source 1..25: mol_type = other DNA
gaacagctcaaagcaatttctacac

SEQ ID NO: 77 - DNA, 24 bp, synthetic construct
  source 1..24: mol_type = other DNA
aatttctacacgagatcctctctc

SEQ ID NO: 78 - DNA, 19 bp, synthetic construct
  source 1..19: mol_type = other DNA
cgactcccaggtacttccg

SEQ ID NO: 79 - DNA, 20 bp, synthetic construct
  source 1..20: mol_type = other DNA
tacttccggaacctgtgctc

SEQ ID NO: 80 - DNA, 21 bp, synthetic construct
  source 1..21: mol_type = other DNA
catgttgcagcaattcactgt

SEQ ID NO: 81 - DNA, 24 bp, synthetic construct
  source 1..24: mol_type = other DNA
caattcactgtaaagctggaaagg

SEQ ID NO: 82 - DNA, 21 bp, synthetic construct
  source 1..21: mol_type = other DNA
gggacagcatcaaatcatcca

SEQ ID NO: 83 - DNA, 22 bp, synthetic construct
  source 1..22: mol_type = other DNA
atcaaatcatccattgcttggg

SEQ ID NO: 84 - DNA, 19 bp, synthetic construct
  source 1..19: mol_type = other DNA
gtcacagacttggctgtcc

SEQ ID NO: 85 - DNA, 18 bp, synthetic construct
  source 1..18: mol_type = other DNA
ttggctgtcccagaatgc

SEQ ID NO: 86 - DNA, 18 bp, synthetic construct
  source 1..18: mol_type = other DNA
atcaacccacagctgcac

SEQ ID NO: 87 - DNA, 17 bp, synthetic construct
  source 1..17: mol_type = other DNA
tgcacagggcaggtctt

SEQ ID NO: 88 - DNA, 17 bp, synthetic construct
  source 1..17: mol_type = other DNA
ccagctgctcaccatcg

SEQ ID NO: 89 - DNA, 20 bp, synthetic construct
  source 1..20: mol_type = other DNA
tcaccatcgctatctgagca

SEQ ID NO: 90 - DNA, 19 bp, synthetic construct
  source 1..19: mol_type = other DNA
tgacaaccacccttaaccc

SEQ ID NO: 91 - DNA, 21 bp, synthetic construct
  source 1..21: mol_type = other DNA
cagttgcaaaccagacctcag

SEQ ID NO: 92 - DNA, 17 bp, synthetic construct
  source 1..17: mol_type = other DNA
gtgcagggtggcaagtg

SEQ ID NO: 93 - DNA, 17 bp, synthetic construct
  source 1..17: mol_type = other DNA
ggcaagtggctcctgac

SEQ ID NO: 94 - DNA, 22 bp, synthetic construct
  source 1..22: mol_type = other DNA
cctttcttgcggagattctctt

SEQ ID NO: 95 - DNA, 21 bp, synthetic construct
  source 1..21: mol_type = other DNA
cggagattctcttcctctgtg

SEQ ID NO: 96 - DNA, 24 bp, synthetic construct
  source 1..24: mol_type = other DNA
aaatattctccatccagtggtttc

SEQ ID NO: 97 - DNA, 21 bp, synthetic construct
  source 1..21: mol_type = other DNA
ccagtggtttcttctttggct

SEQ ID NO: 98 - DNA, 19 bp, synthetic construct
  source 1..19: mol_type = other DNA
gggcatccttgagttccaa

SEQ ID NO: 99 - DNA, 20 bp, synthetic construct
  source 1..20: mol_type = other DNA
cttgagttccaaggcctcat

SEQ ID NO: 100 - DNA, 20 bp, synthetic construct
  source 1..20: mol_type = other DNA
tcaggcccttctgtcttgaa

SEQ ID NO: 101 - DNA, 23 bp, synthetic construct
  source 1..23: mol_type = other DNA
ccttctgtcttgaacatgagttt

SEQ ID NO: 102-103 - skipped (2 entries)

SEQ ID NO: 104 - DNA, 33 bp, synthetic construct; identical to SEQ ID NO: 4
  source 1..33: mol_type = other DNA

SEQ ID NO: 105 - DNA, 33 bp, synthetic construct; identical to SEQ ID NO: 4
  source 1..33: mol_type = other DNA

SEQ ID NO: 106 - DNA, 32 bp, synthetic construct; identical to SEQ ID NO: 5
  source 1..32: mol_type = other DNA

SEQ ID NO: 107 - skipped

SEQ ID NO: 108 - DNA, 19 bp, synthetic construct
  source 1..19: mol_type = other DNA
gactggagttcagacgtgt

SEQ ID NO: 109 - DNA, 20 bp, synthetic construct
  source 1..20: mol_type = other DNA
acactctttccctacacgac

SEQ ID NO: 110 - DNA, 58 bp, synthetic construct
  source 1..58: mol_type = other DNA
aatgatacggcgaccaccgagatctacacnnnnnnnncgacgtaaaacgacggccagt

SEQ ID NO: 111 - DNA, 57 bp, synthetic construct
  source 1..57: mol_type = other DNA
caagcagaagacggcatacgagatnnnnnnnncacacaggaaacagctatgaccatg

SEQ ID NO: 112 - DNA, 66 bp, synthetic construct
  source 1..66: mol_type = other DNA
caagcagaagacggcatacgagatnnnnnnnngtgactggagttcagacgtgtgctcttccgatct

SEQ ID NO: 113 - DNA, 70 bp, synthetic construct
  source 1..70: mol_type = other DNA
aatgatacggcgaccaccgagatctacacnnnnnnnnacactctttccctacacgacgctcttccgatct

SEQ ID NO: 114 - DNA, 40 bp, synthetic construct
  source 1..40: mol_type = other DNA
gagatctacacnnnnnnnncgacgtaaaacgacggccagt

SEQ ID NO: 115 - DNA, 25 bp, synthetic construct
  source 1..25: mol_type = other DNA
catggtcatagctgtttcctgtgtg

SEQ ID NO: 116 - DNA, 45 bp, synthetic construct
  source 1..45: mol_type = other DNA
ggcatacgagatnnnnnnnncacacaggaaacagctatgaccatg

SEQ ID NO: 117 - DNA, 13 bp, synthetic construct
  source 1..13: mol_type = other DNA
gccgtcgttttat